(19) United States
(12) Patent Application Publication
(10) Pub. No.: US 2022/0310200 A1
(43) Pub. Date: Sep. 29, 2022

(54) METHODS FOR IDENTIFYING AND USING DISEASE-ASSOCIATED ANTIGENS

(71) Applicant: KIROMIC BIOPHARMA, INC., Houston, TX (US)

(72) Inventors: Leonardo MIRANDOLA, Plano, TX (US); Maurizio CHIRIVA-INTERNATI, Manvel, TX (US); Zahra TIMSAH, Houston, TX (US); David SPENCER, Houston, TX (US)

(21) Appl. No.: 17/613,191

(22) PCT Filed: May 29, 2020
(86) PCT No.: PCT/US2020/035183

§ 371 Date: Nov. 22, 2021

Related U.S. Application Data
(60) Provisional application No. 62/921,127, filed on May 31, 2019.

(51) Int. Cl. G16B 20/30; G16B 20/20

(52) U.S. Cl. CPC G16B 20/30; G16B 20/20

(57) ABSTRACT

Disclosed here are methods for treating a condition (e.g., cancer) with an appropriate immunotherapeutic agent and/or regimen. Also disclosed are methods for the use of effective combinations of proteins encoded by hot-spot mutations and/or tumor-associated mRNA splice variants to optimize the targeting of a patient's condition (e.g., cancer) with immunotherapies.

### RELATED PATENT APPLICATION(S)

This patent application is a 35 U.S.C. 371 national phase application of International Patent Cooperation Treaty (PCT) Application No. PCT/US2020/035183, filed on May 29, 2020, entitled METHODS FOR IDENTIFYING AND USING DISEASE-ASSOCIATED ANTIGENS, naming Leonardo Mirandola et al. as inventors, and designated by attorney docket no. KIR-1001-PC, which claims the benefit of U.S. provisional patent application No. 62/921,127 filed on May 31, 2019, entitled METHOD FOR THE IDENTIFICATION AND USE OF HOT-SPOT MUTATIONS AND TUMOR-ASSOCIATED SPLICE ISOFORMS IN CANCER IMMUNOTHERAPY, naming Maurizio Chiriva-Internati et al. as inventors, and designated by attorney docket no. KIROMIC-24. The entire content of the foregoing applications is incorporated herein by reference for all purposes, including all text, tables and drawings.

### SEQUENCE LISTING

The instant application contains a Sequence Listing which has been submitted electronically in ASCII format and is hereby incorporated by reference in its entirety. Said ASCII copy, created on Jun. 24, 2020, is named KIR-1001-PC_SL.txt and is 230,128 bytes in size.

### FIELD

The technology relates in part to methods and systems for the identification of disease-associated immunological targets. In certain aspects, the technology relates to a multi-modular system for the identification of disease-associated immunological targets. In certain aspects, the technology relates to a multi-modular system for the identification of peptides preferentially expressed in tumor cells that can trigger an immunological response.

### BACKGROUND

Hot-Spot Mutations in Cancer

In cancer vaccines, white blood cells, and specifically T-cells, are the essential effectors in charge of preventing tumor growth and/or causing its regression. From studies performed in patients who responded to immune checkpoint inhibitors or adoptive cell transfer with TIL (Tumor-Infiltrating Lymphocytes), it became clear that the majority of T-cell responses targeted peptides containing nonsynonymous somatic mutations (Robbins, P. F. et al. Mining Exomic Sequencing Data to Identify Mutated Antigens Recognized by Adoptively Transferred Tumor-reactive T cells. Nature medicine 19, 747-752 (2013); van Rooij, N. et al. Tumor Exome Analysis Reveals Neoantigen-Specific T-Cell Reactivity in an Ipilimumab-Responsive Melanoma. Journal of clinical oncology: official journal of the American Society of Clinical Oncology 31, 10.1200/JCO.2012.1247.7521 (2013); Snyder, A. et al. Genetic Basis for Clinical Response to CTLA-4 Blockade in Melanoma. New England Journal of Medicine 371, 2189-2199 (2014); Tran, E. et al. Immunogenicity of somatic mutations in human gastrointestinal cancers. Science (New York, N.Y.) 350, 1387-1390x(2015); Rizvi, N. A. et al. Mutational landscape determines sensitivity to PD-1 blockade in non—small cell lung cancer. Science (New York, N.Y.) 348, 124-128 (2015); Stevanovic, S. et al. Landscape of immunogenic tumor antigens in successful immunotherapy of virally induced epithelial cancer. Science (New York, N.Y.) 356, 200-205 (2017)). Antigens originating from somatic mutations are collectively known as neoantigens. The discovery of neoantigens and their potential in cancer immunotherapy poses a challenge to developing antigen-targeted immunotherapies. Indeed, given the size of the human exome (around 30 Mbs), the chances that a somatic coding mutation will occur in more than one patient is very small. This means that the majority of neoantigens are patient-specific.

On the other hand, not all somatic mutations occur randomly. Those mutations that affect the function(s) of a protein resulting in promotion of oncogenesis, drug resistance, and tumor revival, collectively known as “driver mutations,” can be systematically detected in multiple patients, and they typically hit constrained “hotspots” sequences of the affected protein. In addition to this, driver mutations typically cause a single or a limited number of amino acid substitutions, which tend to be conserved across the primary tumor and its metastatic sites (Makohon-Moore, A. P. et al. Limited heterogeneity of known driver gene mutations among the metastases of individual patients with pancreatic cancer. Nature genetics 49, 358-366 (2017)). Therefore, if a peptide containing a hotspot mutation is effectively bound by an HLA molecule, this peptide can be effectively used as an “off-the-shelf” neoantigen that can be used with all patients sharing the same mutation and a suitable HLA allele.

Alternative Splicing

A fundamental regulatory process of gene expression is alternative splicing, and it occurs in most human genes (Wang, E. T. et al. Alternative isoform regulation in human tissue transcriptomes. Nature 456, 470-476 (2008)). Alternative splicing is a process by which exons of a gene may be included or excluded in the matured mRNAs and it therefore results in the production of multiple distinct transcript isoforms, generating diverse isoforms of human proteins (Chen, J. & Weiss, W. A. Alternative splicing in cancer: implications for biology and therapy. Oncogene 34, 1-14 (2015)). To date, several mechanisms for alternative splicing have been identified: i) use of alternative promoters, ii) exon skipping, iii) mutually exclusive exons, iv) exon scrambling, v) alternative 5′ and 3′ splice sites, vi) retained introns, and vii) alternative polyadenylation (Wang, E. T. et al. supra; and Chen, J. & Weiss, W. A. supra).

Aberrant Splicing in Cancer

Aberrant splicing patterns are frequently found in neoplastic cells, and they have been associated with splicing regulators (Chen, J. & Weiss, W. A. supra; and Oltean, S. & Bates, D. O. Hallmarks of alternative splicing in cancer. Oncogene 33, 5311-5318 (2014)). Dysregulated expression of splicing regulators such as RBFOX2, PTB/PTBP1, and SRSF1, can cause splicing pattern changes in multiple genes (Oltean, S. & Bates, D. O. supra; and Danan-Gotthold, M. et al. Identification of recurrent regulated alternative splicing events across human solid tumors. Nucleic acids research 43, 5130-5144 (2015)).

The advent of whole transcriptome sequencing (RNA-seq) and development of related bioinformatics analysis tools have enabled researchers to detect and measure not only the expression of genes but also their sequences and structural configurations. When applied to cancer, disease-specific formation of alternative transcripts could be identified as potential biomarkers for diagnosis (Danan-Gotthold, M. et al. supra; and Barrett, C. L. et al. Systematic transcriptome analysis reveals tumor-specific isoforms for ovarian cancer diagnosis and therapy. Proceedings of the National Academy of Sciences of the United States of America 112, E3050-3057 (2015)) and cancer stratification (Eswaran, J. et al. RNA sequencing of cancer reveals novel splicing alterations. Scientific reports 3, 1689 (2013); and Zhao, Q. et al. Tumor-specific isoform switch of the fibroblast growth factor receptor 2 underlies the mesenchymal and malignant phenotypes of clear cell renal cell carcinomas. Clinical cancer research: an official journal of the American Association for Cancer Research 19, 2460-2472 (2013)).

### SUMMARY

Provided herein is an Immunotherapy Builder System (IBS) that includes multiple modules. Also provided herein are methods for implementing an IBS and/or one or more modules of an IBS. In certain implementations, an IBS can narrow a multitude of amino acid sequence variants to a subset of predicted disease-associated variants. In certain implementations, an IBS can narrow a multitude of amino acid subsequences in an input amino acid sequence to a subset identified as having immunogenic potential (e.g., for an immunotherapy). An IBS can facilitate in silico (i) discovery of novel disease-associated targets, and/or (ii) narrowing of a large number of targets to a significantly smaller subset of targets having high immunogenic potential, thereby facilitating resource-efficient development of novel immunotherapies. Disease-associated targets (e.g., disease associated amino acid sequence variants) predicted by systems and processes described herein can be considered predicted disease-associated antigens. Portions of disease-associated targets (e.g., amino acid subsequences) identified by systems and processes described herein as having immunogenic potential, and/or longer amino acid sequences each containing one or more of such portions, can be considered predicted disease-associated antigens. Portions of predicted disease-associated targets can be identified by systems and processes described herein as having immunogenic potential according to assessment of major histocompatibility complex (MHC) interaction and/or T-cell receptor (TCR) interaction and/or B-cell receptor (BCR) interaction, for example.

An IBS can include two or more of the following modules: a Differential Expression Module (DEM), a MHC Allele Affinity Determination Module (MAAM), a MHC Composite Feature Module (MCFM), a MHC Fragment Locator Module (MFLM), a T-Cell Receptor Immunogenicity Determination Module (TIM); and a B-Cell Receptor Epitope Determination Module (BEM). An IBS can include a Sequence Acquisition Interface (SAI), which can be implemented to acquire an amino acid sequence of interest. A MAAM, MCFM, MFLM, BEM and/or a DEM can be configured to receive an amino acid sequence from an SAI.

An IBS can be implemented to (i) identify a disease-associated amino acid sequence variant (e.g., by implementation of a DEM) among variants encoded by a particular gene; and/or (ii) compute MHC binding affinity values for amino acid subsequences within an amino acid sequence of interest (e.g., by implementation of a MAAM, MCFM and/or a MFLM); and/or (iii) compute a T-cell receptor (TCR) immunogenicity score for each of a plurality of amino acid subsequences having an estimated MHC binding affinity value above or below a threshold (e.g., by implementation of a TIM); and/or (iv) identify B-cell receptor (BCR) epitopes in an amino acid sequence of interest (e.g., by implementation of a BEM), for example.

A MAAM can compute a MHC binding affinity value for amino acid subsequences of an input amino acid sequence, for one or more MHC alleles or MHC supertypes, by implementation of a convolutional neural network (CNN) that contains a plurality of virtual neurons arranged in capsules. A MAAM can compute a MHC binding affinity value with an advantageously low error rate, and can narrow amino acid subsequences to a subset predicted to exhibit strong and/or intermediate MHC binding affinity. These features are useful for identifying a subset of amino acid subsequences of high immunogenic potential for an immunotherapy, for example.

For amino acid subsequences outputted by a MAAM, a MFLM can output a graphic representation of the amino acid subsequences, or subset thereof, mapped to an input amino acid sequence. These features are useful for narrowing amino acid subsequences to a subset located in one or more regions in the amino acid sequence that can be presented by multiple MHC alleles. Such a narrowing process is useful for building an immunotherapy that is potentially effective across a broad population, for example.

A MCFM can compute a composite MHC binding affinity value for amino acid subsequences of an input amino acid sequence, for one or more MHC alleles or MHC supertypes, where the composite binding affinity value is based on (i) a proteasome cleavage score for the amino acid subsequence, and/or (ii) a transporter affinity score for the amino acid subsequence, and/or (iii) a MHC allele or MHC supertype binding affinity value for the amino acid subsequence (e.g., a normalized MHC allele or MHC supertype binding affinity value). A composite binding affinity value is useful for narrowing amino acid subsequences to a subgroup having high immunogenic potential for an immunotherapy, for example.

A TIM can compute a T-cell receptor (TCR) immunogenicity score based on estimation of interaction of amino acid subsequences of an input amino acid with a TCR. A TIM often is implemented after a subset of amino acid subsequences characterized by high immunogenicity potential and/or high multiple MHC allele-binding potential is identified by one or more modules that assess MHC interaction (i.e., a MAAM, a MFLM and/or a MCFM). Immunogencity scores computed by a TIM are useful for narrowing amino acid subsequences to a smaller subset of high T-cell-mediated immunogenic potential, for example.

A BEM can compute a B-cell receptor (BCR) epitope score for each amino acid in an input amino acid sequence, where the score is indicative of the probability that the amino acid exists within a BCR epitope. scores computed by a BEM are useful for narrowing amino acid subsequences to a smaller subset of high B-cell-mediated immunogenic potential, for example.

A DEM can identify a disease-associated amino acid sequence variant among variants encoded by a particular gene based on an analysis of expression level of the variant in disease samples and non-disease samples from multiple tissues. A DEM is useful for identifying disease-associated alternatively-spliced variants. In certain instances, a disease-associated alternatively-spliced variant includes an insert of an amino acid or two or more consecutive amino acids relative to other variants encoded by a gene, for example. A DEM is useful for identifying disease-associated variants that can be targeted by an immunotherapeutic, for example. An amino acid sequence of a disease-associated variant identified by a DEM, or portion thereof, can be utilized as an input amino acid sequence for one or more of a MAAM, a MFLM, a MCFM, a TIM and a BEM, for example.

Certain embodiments are described further in the following description, examples, claims and drawings.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIGS. 1A-1B show NKAIN1 splice variant differential expression analysis.

FIG. 2 shows qRT-PCR differential expression analysis of tumor-associated splice variant in breast cancer versus normal breast samples. The horizontal line (just below Ct 33) marks the cut-off for positivity, determined as the mean normal tissue Ct—2× normal tissues standard deviation.

FIGS. 3A-3D show MSLN splice variant differential expression analysis. FIG. 3A shows an example graphical interface using a search gene option (top panel) and a multi-sequence alignment (bottom panel) for the MSLN isoform (unique amino acid sequence is PQAPRRPL (SEQ ID NO:19), tumor isoform highlighted and in bold (fifth sequence from top; a portion of SEQ ID NO:18, identified as SEQ ID NO: 99), canonical (second sequence from top; a portion of SEQ ID NO:17, identified as SEQ ID NO: 98), and five minor splice variants (first, third, fourth, sixth, and seventh sequences top; portions of SEQ ID NOs: 50-54, respectively, each identified as SEQ ID NO: 98). FIG. 3B shows MSLN splice variant differential expression analysis (normal samples). FIG. 3C shows MSLN splice variant differential expression analysis (normal samples). FIG. 3D shows MSLN splice variant differential expression analysis (tumor samples).

FIG. 4 shows qRT-PCR differential expression analysis of tumor-associated splice variant in ovarian cancer versus normal ovary samples. The horizontal line (between Ct 31 and Ct 33) marks the cut-off for positivity, determined as the mean normal tissue Ct—2× normal tissues standard deviation.

FIG. 5 shows results for a cytotoxicity assay.

FIGS. 6A-6C show UPK3B splice variant differential expression analysis. FIG. 6A shows UPK3B splice variant differential expression analysis (normal samples). FIG. 6B shows UPK3B splice variant differential expression analysis (normal samples). FIG. 6C shows UPK3B splice variant differential expression analysis (tumor samples). BLCA, bladder urothelial carcinoma; MESO, mesothelioma; OV, ovarian serious cystadenocarcinoma.

FIG. 7 shows sequence and topology of a UPK3B variant. Panel A: protein sequences from different transcripts are compared and a portion of the alignment is shown. The IsoUPK3B is shown on top (a portion of SEQ ID NO:47, identified as SEQ ID NO: 100), the canonical UPK3B is shown in the middle (a portion of SEQ ID NO:46, identified as SEQ ID NO: 101), and a minor splice variant is shown on the bottom (a portion of SEQ ID NO.48, identified as SEQ ID NO: 101). Panel B: SACS MEMSAT2 Transmembrane Prediction Tool was utilized to predict the protein topology of the IsoUPK3B amino acid sequence (residues 1-272 of SEQ ID NO:47). A fragment of the unique peptide, circled (WSDPITLHQGK (SEQ ID NO:27), which is a portion of SEQ ID NO:49), is in the extracellular protein domain. Panel C: protein sequences from different transcripts are compared and a portion of the alignment is shown (a larger portion compared to Panel A). The canonical UPK3B is shown on top (a portion of SEQ ID NO:46, identified as SEQ ID NO: 102), the IsoUPK3B is shown in the middle (a portion of SEQ ID NO:47, identified as SEQ ID NO: 103), and a minor splice variant is shown on the bottom (a portion of SEQ ID NO.48, identified as SEQ ID NO: 102).

FIG. 8 shows detection of IsoUPK3B peptide, WSDPITLHQGK (SEQ ID NO:27) with Proteomics tools. The peptide WSDPITLHQGK (SEQ ID NO:27), which includes a fragment of the unique peptide of IsoUPK3B, was detected in 77% of OV tumor samples and in 78% of adjacent normal samples. No other UPK3B were detected. The box plots illustrate the distribution of WSDPITLHQGK (SEQ ID NO:27) in tumor samples and adjacent normal tissues, with tumor>normal.

FIG. 9 shows TNFRSF13B isoform differential expression analysis.

FIG. 10 shows sequence and topology of an TNFRSF13B isoform. Panel A: protein sequences from different transcripts are compared and a portion of the alignment is shown. The IsoTNFRSF13B is shown in on top (a portion of SEQ ID NO:29, identified as SEQ ID NO: 104), the canonical TNFRSF13B is shown in the middle (a portion of SEQ ID NO:30, identified as SEQ ID NO: 105), and a minor splice variant is shown on the bottom (a portion of SEQ ID NO:31, identified as SEQ ID NO: 104). Panel B: SACS MEMSAT2 Transmembrane Prediction Tool was utilized to predict the protein topology. The unique peptide (SEQ ID NO: 29), shaded region (SEQ ID NO:32), is in the extracellular protein domain.

FIG. 11 shows relative expression of the TNFRSF13B isoform plotted as fold change expression over the average obtained from the non-tumor samples.

FIG. 12 shows an example graphical interface for a DEM. The peptides in the third column from the left (listed from top to bottom) are SEQ ID NOs: 55-70, respectively.

FIG. 13 shows an example graphical interface for a DEM.

FIG. 14 shows an example graphical interface for a DEM. The peptides in the third column from the left (listed from top to bottom) are SEQ ID NOs: 55-70, respectively.

FIG. 15 shows an example graphical interface for a DEM.

FIG. 16 shows an example graphical interface for a DEM.

FIG. 17 shows an example graphical interface for a MAAM. The peptides in the third column from the left (listed from top to bottom) are SEQ ID NOs: 55-70, respectively.

FIG. 18 shows an example graphical interface for a MCFM. The peptides in the first column (listed from top to bottom) are SEQ ID NOs: 71-75, respectively.

FIG. 19 shows an example graphical interface for a MFLM. The peptides in the sixth column from the left (listed from top to bottom) are SEQ ID NOs: 76-91, respectively.

FIG. 20 shows an example graphical interface for a MFLM. The example polypeptide shown in the heat map is SEQ ID NO:92.

FIG. 21 illustrates an implementation of an IBS.

### DETAILED DESCRIPTION

Immunotherapy Builder System (IBS)

An Immunotherapy Builder System (IBS) is a graphical multi-purpose computational platform that can identify new disease-associated (e.g., cancer-associated) immunological targets as those segments of proteins that are present only, or preferentially, in tumor cells because of tumor-specific mutations or tumor-associated use of mRNA transcripts derived from alternative splicing events. An IBS can include multiple modules. An IBS can include two or more of the following modules: a Differential Expression Module (DEM), a MHC Allele Affinity Determination Module (MAAM), a MHC Composite Feature Module (MCFM), a MHC Fragment Locator Module (MFLM), a T-Cell Receptor Immunogenicity Determination Module (TIM); and a B-Cell Receptor Epitope Determination Module (BEM).

In certain instances, a MAAM and/or a MFLM of an IBS identifies peptides binding to a given set of HLA alleles by calculating their binding affinities. In certain instances, a MCFM of an IBS identifies peptides binding to a given set of HLA molecules by calculating their binding affinities, the likelihood of proteasome processing, and the likelihood of TAP binding. In certain instances, implementation of one or more of a MAAM, MCFM, MFLM and TIM of an IBS identifies peptides that are predicted to bind to the TCR of CD4+ T-cells. In certain instances, implementation of one or more of a MAAM, MCFM, MFLM and TIM of an IBS identifies peptides that are predicted to bind to the TCR of CD8+ T-cells. In certain instances, a DEM of an IBS is a computational platform that can analyze and compare mutations and transcript isoform expression across multiple tumor tissues and compare those to healthy tissues would be the tool necessary to identify those mutations and isoforms that are exclusively or preferentially expressed by cancer cells compared with non-tumor cells. In another embodiment, an IBS is a computational platform that can select the immunogenic peptides containing a cancer-specific mutation or derived from a cancer-associated splice isoform. In another embodiment, tumor-specific hot-spot mutations are defined as single amino acid substitutions or amino acid insertions, or amino acid deletions, that occur at least in 5%, or 10%, or 25%, or 50%, or 75%, or 90%, of cases of a given tumor type. Additional descriptions and embodiments of IBS modules are provided below.

Differential Expression Module (DEM)

A particular gene can give rise to different polypeptide or peptide variants according to one or more alterations at the DNA level (e.g., point mutation event) or mRNA level (e.g., alternative splice event). A particular gene can encode two or more polypeptide variants or peptide variants that are distinguished, for example, by at least one single amino acid substitution, at least one single amino acid insertion, at least one single amino acid deletion, at least one substitution of two or more consecutive amino acids, at least one insertion of two or more consecutive amino acids, at least one deletion of two or more consecutive amino acids, or a combination thereof.

Methods described herein may include an expression analysis of a variant subsequence. In some embodiments, a method described herein includes an analysis of differential expression (e.g., expression of a gene or variant subsequence in different organs or tissues, expression of a gene or variant subsequence in different subjects, expression of a gene or variant subsequence in healthy vs. disease organs or tissues, expression of a gene or variant subsequence in healthy vs. disease subjects). In some embodiments, a differential expression analysis includes comparing gene or variant subsequence expression in tumors vs. surrounding tissue (e.g., in the same subject). In some embodiments, a differential expression analysis includes comparing gene or variant subsequence expression in tumors (e.g., from one or more disease subjects) vs. corresponding tissue (e.g., from one or more healthy subjects). Corresponding tissue generally refers to an equivalent organ or tissue in a healthy subject that is cancerous in a disease subject. For example, if a disease subject has prostate cancer, corresponding tissue would refer to prostate tissue from a healthy subject. Any suitable method for determining or measuring levels of gene or variant subsequence expression may be used in a gene or variant subsequence expression analysis and/or a differential expression analysis. Examples of methods for measuring expression levels include qPCR, RT-qPCR, RNA-Seq, microarray, northern blot, differential display, and RNase protection assay.

In some embodiments, expression levels may be measured using a quantifiable amplification method. For example, expression levels may be measured using a quantitative PCR (qPCR) approach (e.g., on cDNA generated from mRNA from a sample), or a reverse transcriptase quantitative PCR (RT-qPCR) approach (e.g., on mRNA from a sample). Quantitative PCR (qPCR), which also may be referred to a real-time PCR, monitors the amplification of a targeted nucleic acid molecule during a PCR reaction (i.e., in real time). This method may be used quantitatively (quantitative real-time PCR) and semi-quantitatively (i.e., above/below a certain amount of nucleic acid molecules; semi-quantitative real-time PCR. Methods for qPCR include use of non-specific fluorescent dyes that intercalate with double-stranded DNA, and sequence-specific DNA probes labelled with a fluorescent reporter, which generally allows detection after hybridization of the probe with its complementary sequence. Quantitative PCR methods typically are performed in a thermal cycler with the capacity to illuminate each sample with a beam of light of at least one specified wavelength and detect the fluorescence emitted by an excited fluorophore.

For non-specific detection, a DNA-binding dye binds to all double-stranded (ds) DNA during PCR. An increase in DNA product during PCR therefore leads to an increase in fluorescence intensity measured at each cycle. For qPCR using dsDNA dyes, the reaction typically is prepared like a basic PCR reaction, with the addition of fluorescent dsDNA dye. Then the reaction is run in a real-time PCR instrument, and after each cycle, the intensity of fluorescence is measured with a detector (the dye only fluoresces when bound to the dsDNA (i.e., the PCR product)). In certain applications, multiple target sequences may be monitored in a tube by using different types of dyes. For specific detection, fluorescent reporter probes detect only the DNA containing the sequence complementary to the probe. Accordingly, use of the reporter probe increases specificity, and enables performing the technique even in the presence of other dsDNA. Using different types of labels, fluorescent probes may be used in multiplex assays for monitoring several target sequences in the same tube. This method typically uses a DNA-based probe with a fluorescent reporter at one end and a quencher of fluorescence at the opposite end of the probe. The close proximity of the reporter to the quencher prevents detection of its fluorescence. During PCR, the probe is broken down by the 5′ to 3′ exonuclease activity of the polymerase, which breaks the reporter-quencher proximity and thus allows unquenched emission of fluorescence, which can be detected after excitation with a laser. An increase in the product targeted by the reporter probe at each PCR cycle therefore causes a proportional increase in fluorescence due to the breakdown of the probe and release of the reporter.

In some embodiments, expression levels may be measured using a sequencing process (e.g., RNA sequencing (RNA-Seq)). RNA-Seq typically uses high-throughput sequencing to detect the presence and/or measure the quantity of RNA in a sample. In certain applications, RNA-Seq allows for detection and/or measurement of alternative spliced transcripts, post-transcriptional modifications, gene fusion, mutations/SNPs, changes in gene expression over time, and/or differential expression. RNA-Seq can analyze different populations of RNA, which may include mRNA transcripts, total RNA, small RNA (e.g., miRNA), tRNA, and ribosomal RNA. RNA-Seq also may be used to assess exon/intron boundaries.

A Differential Expression Module (DEM) can identify a disease-associated polypeptide or peptide variant of a particular gene. Stated another way, a DEM can identify a disease-associated variant of a particular gene, and an amino acid sequence of one or more variants of the gene may be aligned and/or outputted by a DEM. In certain instances, measuring expression levels are not part of a process implemented by a DEM. In certain instances expression level values are within a dataset within a module described herein or received by a module described herein (e.g., a DEM). In certain instances a dataset is received and/or stored in memory, and sometimes a dataset is in a database. A dataset within, or received by, a module described herein (e.g., a DEM) can contain expression level values for transcripts in disease samples and non-disease samples from multiple tissues. Transcripts in a dataset can be virtual RNA transcripts (e.g., mRNA transcripts) and representative polynucleotide sequences (e.g., RNA, DNA and/or cDNA sequences) of transcripts or portions thereof can be included in a dataset. Transcripts in a dataset can correspond to amino acid sequence variants encoded by a gene, and a database can include representative amino acid sequences and/or amino acid subsequence corresponding to (e.g., translated from) transcripts or portions thereof. Transcripts can correspond to sequence variants encoded by one or more genes, and a database can include expression level values associated with polynucleotide sequences, amino acid sequences and/or amino acid subsequence corresponding to transcripts or portions thereof.

A DEM can include or can receive a dataset containing amino acid sequences of polypeptides and peptides encoded by genes and associated expression level information. If there are three polypeptide variants for a particular gene, for example, the DEM can include or receive (i) an amino acid sequence for each variant, and (ii) associated expression level information for each variant. Amino acid sequence information and associated expression level information can be stored in a DEM in any suitable format (e.g., a .tar archive). Expression level information stored in a DEM can exist in a DEM in any suitable manner, and sometimes exists as normalized expression level information. Expression level information can exist, for example, as transcripts per million (TPM) values, fragments per kilobase per million reads mapped (FPKM) values, reads per kilobase per million reads mapped (RPKM) values, RNA-seq by expectation-maximization (RSEM) values, or combination of such values, in a DEM. Transcripts per million (TPM) values, for example, are normalized expression level values, and a TPM value for a particular gene/transcript represents the number of RNA molecules in a sample for every one million RNA molecules in the sample. A TPM value generally is determined for RNA-seq samples. Expression level information can exist in a DEM, for example, as average expression level values. For example, expression level information can exist in a DEM as average TPM values, average FPKM values, average RPKM values, average RSEM values, or combination thereof. As used herein, an average value can be a mean, median, mode value.

Candidate disease-associated polypeptides or peptide variants (e.g., tumor-specific isoforms) may be defined by comparing the isoform expression levels between normal and disease (e.g., tumor) conditions. In one embodiment, candidate tumor-specific isoforms are defined by comparing the isoform expression levels between normal and tumor conditions, by calculating the median TPM-based fold change, and the presence or absence of one candidate tumor-specific isoform. In another embodiment, candidate tumor-specific isoforms are defined by comparing isoform expression levels between normal and tumor conditions, by calculating the median FPKM-based fold change, and the presence or absence of one candidate tumor-specific isoform. In another embodiment, candidate tumor-specific isoforms are defined by comparing isoform expression levels between normal and tumor conditions, by calculating the median RPKM-based fold change, and the presence or absence of one candidate tumor-specific isoform. In another embodiment, candidate tumor-specific isoforms are defined by comparing isoform expression levels between normal and tumor conditions, by calculating the median RSEM-based fold change, and the presence or absence of one candidate tumor-specific isoform.

A dataset of a DEM, or a dataset received by a DEM, can include (i) expression level information associated with amino acid sequence variants for disease samples, and (ii) expression level information associated with amino acid sequence variants for non-disease samples. A dataset can include expression levels (e.g., average expression levels) of transcripts associated with particular amino acid sequence variants for disease samples. A dataset can include expression levels (e.g., average expression levels) of transcripts associated with particular amino acid sequence variants for non-disease samples. A dataset can include a composite expression level associated with a particular amino acid sequence variant for non-disease samples (e.g., for all non-disease samples). A composite expression level often is an average of the average expression level in a dataset for each tissue of origin of non-disease samples (e.g., all non-diseased samples in a dataset).

Each disease sample in a dataset of a DEM can be associated with (i) a tissue of origin, and (ii) matched expression level values for each transcript derived from non-disease tissue adjacent to the disease tissue. Sample information can be from any suitable dataset or combined dataset, a non-limiting example of which includes a datasets from TOGA (https://www.cancer.gov/about-nci/organization/ccg/research/structural-genomics/tcga).

A disease sample generally is from a portion of a tissue of an organism identified as being diseased, and a non-disease sample generally is from a portion of a tissue of an organism identified as not being diseased. A disease sample and a non-disease sample sometimes are from the same subject, and sometimes are not from the same subject. A disease sample sometimes is from a portion of a tissue of an organism identified as being diseased, and a non-disease sample sometimes is from a portion of an adjacent tissue of an organism identified as not being diseased. For example, a disease sample sometimes is from a cancer tumor and a non-disease sample sometimes is from a non-tumor tissue adjacent to the tumor in the same subject.

A disease sometimes is a condition, and a disease or condition sometimes is diagnosed, inferred or suspected for a subject. Non-limiting examples of disease samples include samples from subjects having or suspected of having Alzheimer's disease, Parkinson's disease, Lupus, IPEX syndrome, diabetes, rheumatoid arthritis, influenza, pneumonia, or tuberculosis. A disease sample can be a cancer sample, and non-limiting examples of cancer samples include samples from subjects having or suspected as having acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, chronic myeloid leukemia, acute lymphocytic leukemia, multiple myeloma, non-Hodgkin lymphoma, Hodgkin lymphoma, marginal zone lymphoma, follicular lymphoma, small lymphocyte lymphoma, B-cell lymphoma, diffuse large B-cell lymphoma or mantle cell lymphoma

A DEM can compare, for a selected disease: (i) an expression level (e.g., average expression level) of a particular amino acid sequence variant for disease samples, to (ii) a composite expression level of the particular amino acid sequence variant for non-disease samples (e.g., for all non-disease samples). A DEM can compare, for a selected gene: (i) an expression level (e.g., average expression level) of a particular amino acid sequence variant for disease samples, to (ii) a composite expression level of the particular amino acid sequence variant for non-disease samples (e.g., for all non-disease samples).

A DEM can perform several computations using expression level information in a dataset. A DEM can, for example: (a) compute an average expression level value for each transcript for disease samples; and/or (b) compute for each amino acid sequence variant a “related variant” value for disease samples and a “related variant” value for non-disease samples, where the “related variant” value is (i) the average (e.g., mean or median) expression level for the variant, divided by (ii) the sum of average expression level values for each variant of the gene; and/or (c) compute for each amino acid sequence variant a “fold change” value, where the “fold change” value is (i) the average expression level for the amino acid sequence variant in disease samples, divided by (ii) the average expression level for the amino acid sequence variant in non-disease samples. The “related variant” value can be expressed as a percentage referred to as an “expression percentage.” A computation described for part (a) also can include matching each average expression level value for each transcript with (i) a composite average expression level for the transcript for all non-disease samples, and/or (ii) a highest tissue expression level identified from all non-disease samples for the transcript. While each of the related variant value and the fold change value described in part (b) and part (c) is computed by dividing (i) by (ii), each ratio independently may be computed by dividing (ii) by (i).

After performing computations (a) and/or (b) and/or (c) described in the preceding paragraph, a DEM can generate a “disease sample only” variant list. Each variant selected for the list is expressed in disease samples but generally not expressed in non-diseased samples, where “not expressed” is defined as an expression level (e.g., TPM expression level) of less than 0.00001. The resulting list often is sorted by expression level value such that the most highly-expressed “disease sample only” variants are at the top of the list. A configurable cutoff value can be applied to expression level in disease samples, whereby only amino acid variants are displayed that are associated with a value greater than or equal to the value associated with the cutoff. For example, a threshold of greater-than-or-equal-to a TPM expression level of 1.0, and an expression percentage of greater than 10% can be applied.

After performing computations (a) and/or (b) and/or (c) described above, a DEM can generate a “disease sample specific” variant list. Each variant selected for the list can be (i) the dominant variant in disease samples, and/or (ii) not the dominant variant in non-disease samples.

After performing computations (a) and/or (b) and/or (c) described above, A DEM can generate a “disease upregulated” variant list. Each variant selected for the list exhibits a fold change value equal-to-or-greater-than a threshold value. The fold change expression level threshold can be configurable by a user, and a threshold value can be a two-fold threshold value, for example.

A DEM can generate a multi-sequence alignment (MSA) for each variant included in a list, which facilitates identification of an amino acid subsequence present in a particular variant that is not present in at least one other variant of a particular gene. A MSA often aligns a variant amino acid sequence with an amino acid sequence of at least one other variant encoded by the same gene. A MSA can be generated using any suitable sequence alignment algorithm, non-limiting examples of which include Clustal (e.g., ClustalW, ClustalW2, Clustal Omega), Multiple Alignment using Fast Fourier Transform (MAFFT), T-COFFEE, M-COFFEE, LALIGN, PSAlign, PRRN, PRRP, DIALIGN, MUSCLE, MergeAlign, Partial-Order Alignment (POA), Sequence Alignment and Modeling System (SAM), HMMER, PRANK, PAGAN, ProGraphMSA, MEME, MAST and EDNA. A DEM can generate a MSA based on a gene identifier, which can involve synching amino acid sequence databases having disparate gene identifier information. A non-limiting example of such a synching process is described hereafter. A TCGA variant model was based on the hg19 2009 version of the UCSC gene dataset (gene models built by UCSC as part of a genome browser). The UCSC table known as GenePep was downloaded for hg19 to obtain the protein sequence of each of the variants used in the TCGA analysis. Multiple versions of the hg19 USC gene models were released over time; the UCSC gene hg19 version 12 from 2009 was obtained that matched the gene models used in the TCGA analysis. A gene symbol scheme was constructed for transcript mapping from the TCGA reference dataset and the GenePep table was used to build a Sqlite database that supports looking up a gene symbol and returning all the protein sequences of the transcripts of the gene. These were then run through a multi-sequence alignment program and then through a format routine. The resulting structure allowed for a protein alignment program to receive a gene symbol and produces a multi-sequence alignment that can be used to identify sections specific to a tumor-associated variant.

A DEM can generate a box plot of expression level values for non-disease samples by tissue for a given variant. Data from any suitable dataset can be utilized for such a box plot, non-limiting examples of which include the TCGA dataset described herein and the GTEX dataset (https://www.gtexportal.org/home/). A DEM can generate a box plot of expression level values of a variant in disease samples for different tissues, and can generate: (i) upper whisker, lower whisker, upper quartile, lower quartile, and/or an average of the distribution of values for a selected disease type; and/or (ii) a maximum expression level value (e.g., maximum TPM value) for a non-disease sample from a relevant tissue

A DEM can include an interface (e.g., a graphic interface) that facilitates selection of (i) a gene of interest according to a gene identifier (e.g., gene name, a gene tag (i.e., an abbreviated version of a gene name), accession number), or (ii) a tissue type of interest (e.g., TCGA tissue type of interest). An interface can facilitate selection of one or more filters that enhance identification of variants that are immunologic and/or can be immunologically targeted. An interface can facilitate selection of an expression level threshold filter that permits listing of only variants associated with a minimum disease sample expression level (e.g., minimum disease sample TPM). An interface can facilitate input of an expression level threshold filter that permits listing only of variants associated with a maximum non-disease sample expression level (e.g., maximum non-disease sample TPM). An interface can facilitate selection of a filter that permits listing of only variants of genes that encode a cell-surface polypeptide (e.g., based on an annotation in a dataset specifying that a particular gene encodes a cell-surface polypeptide). An interface can facilitate selection of a filter that permits listing of only variants having at least one insertion of a single amino acid or two or more consecutive amino acids relative to a canonical amino acid sequence.

An interface of a DEM can display a list of variants. An interface can output a sorted list of variants with the strongest disease/non-disease differences at the top of the list. Two types of lists are available for each tissue: (i) variants expressed in disease samples but not expressed in non-disease samples, and (ii) variants expressed at significantly higher levels in disease samples than non-disease samples. An interface also can allow selection of a particular variant in a list for display of additional output pertaining to the variant. For example, additional output can include (i) a detail panel showing a multi-sequence alignment for the selected variant and all other variants encoded by the gene; and/or (ii) a box plot of expression of the variant in non-disease samples for different tissues (e.g., a box plot for TOGA dataset and/or a box plot for GTEX dataset). Additional output also can include a box plot of expression level values for the variant in disease samples for different tissues, which optionally can include (i) upper whisker, lower whisker, upper quartile, lower quartile, and/or an average of the distribution of values in a selected disease type; and/or (ii) a maximum expression level value (e.g., maximum TPM value) for a non-disease sample from a relevant tissue.

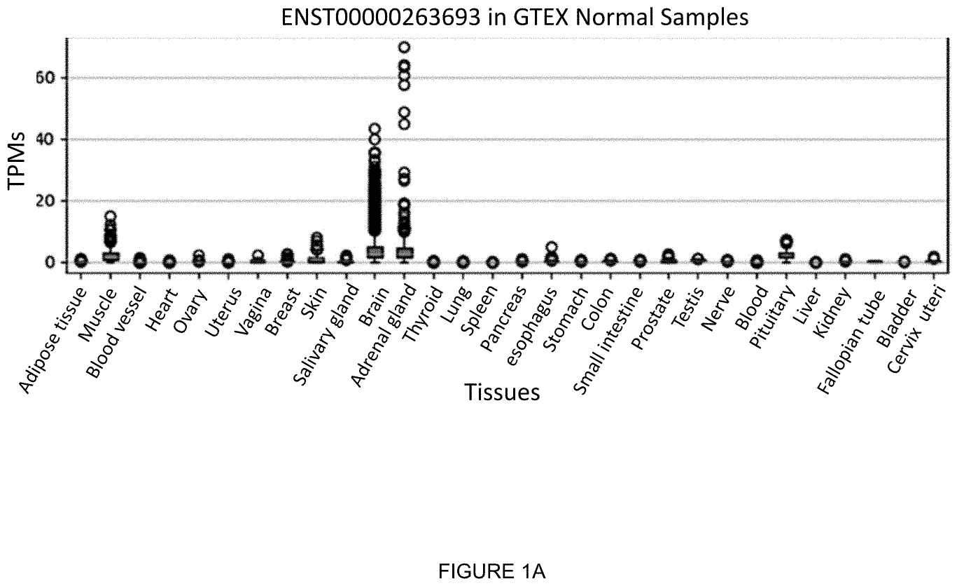

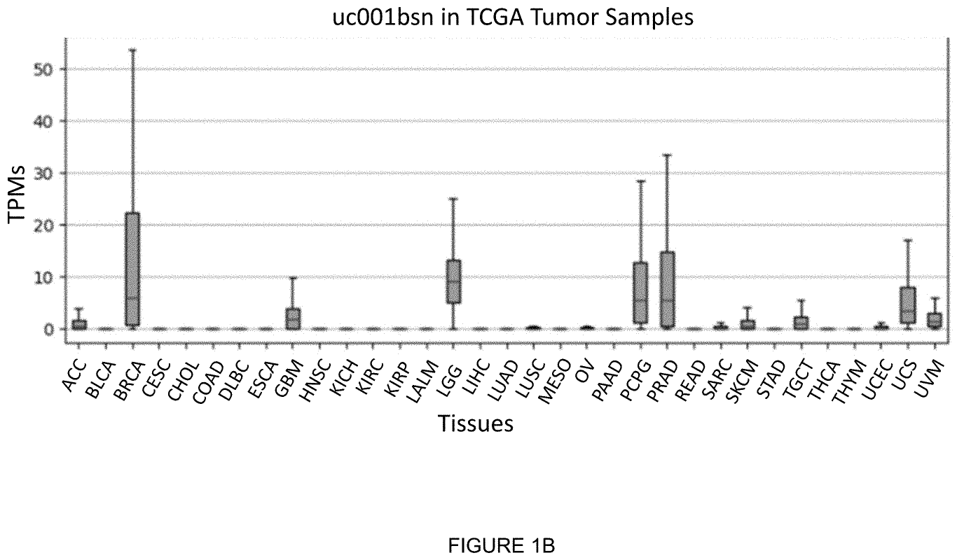

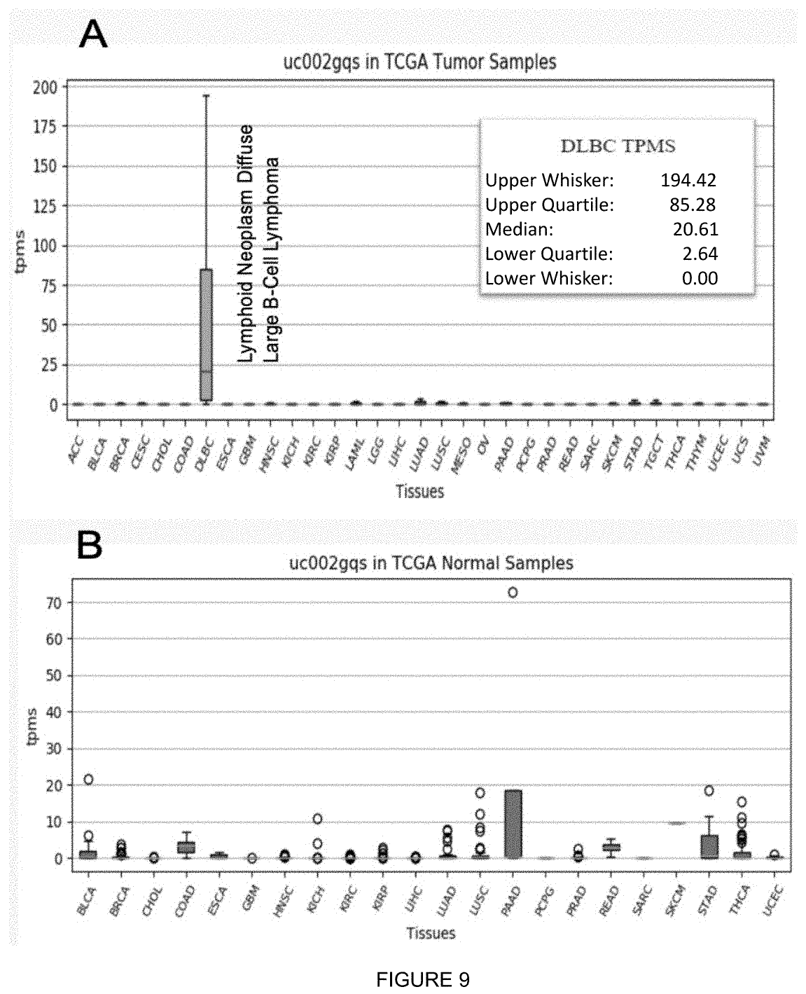

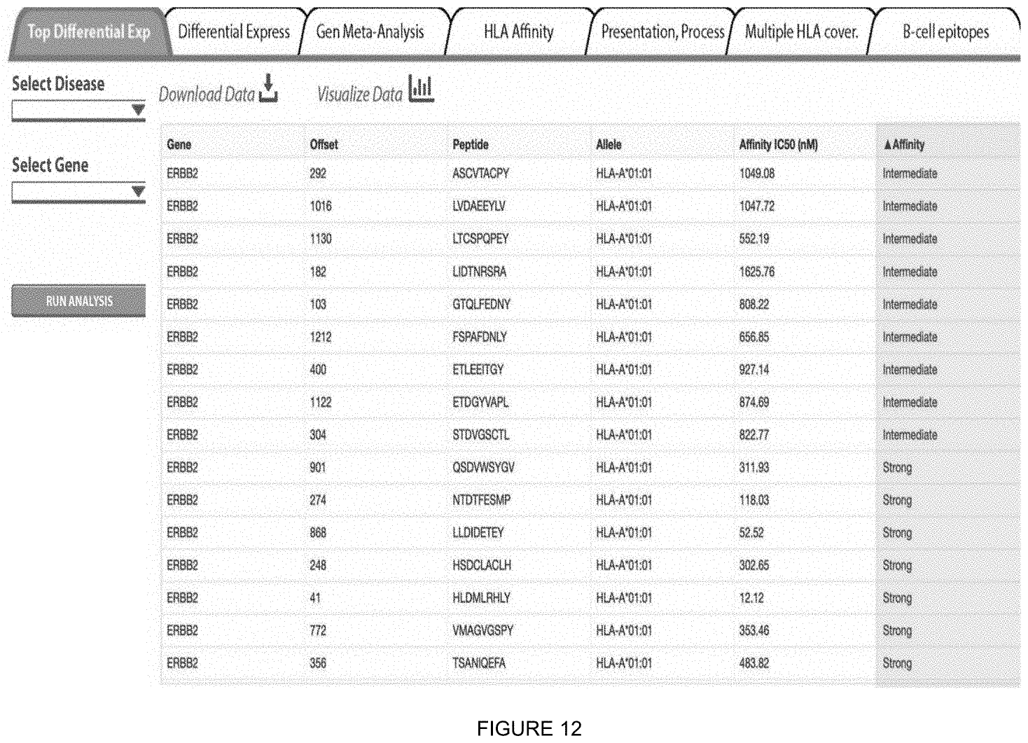

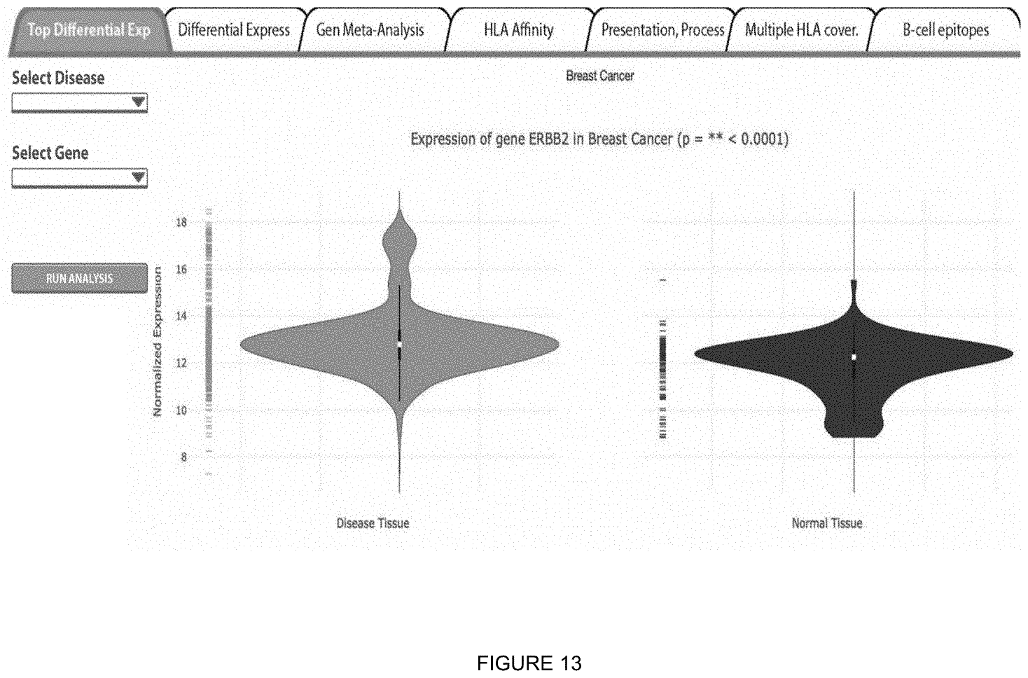

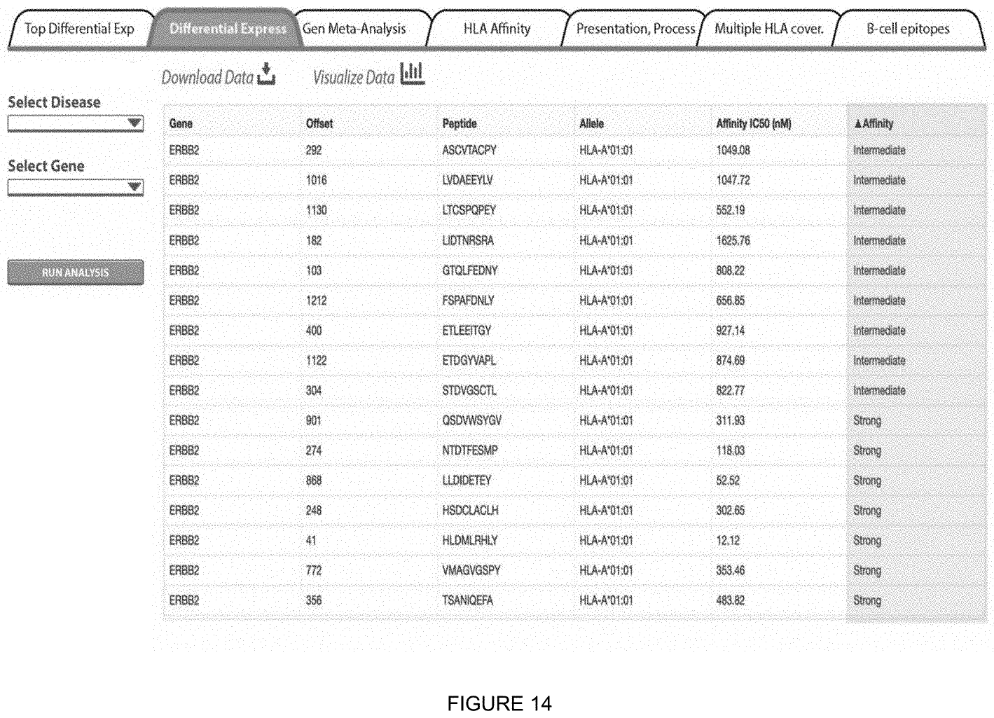

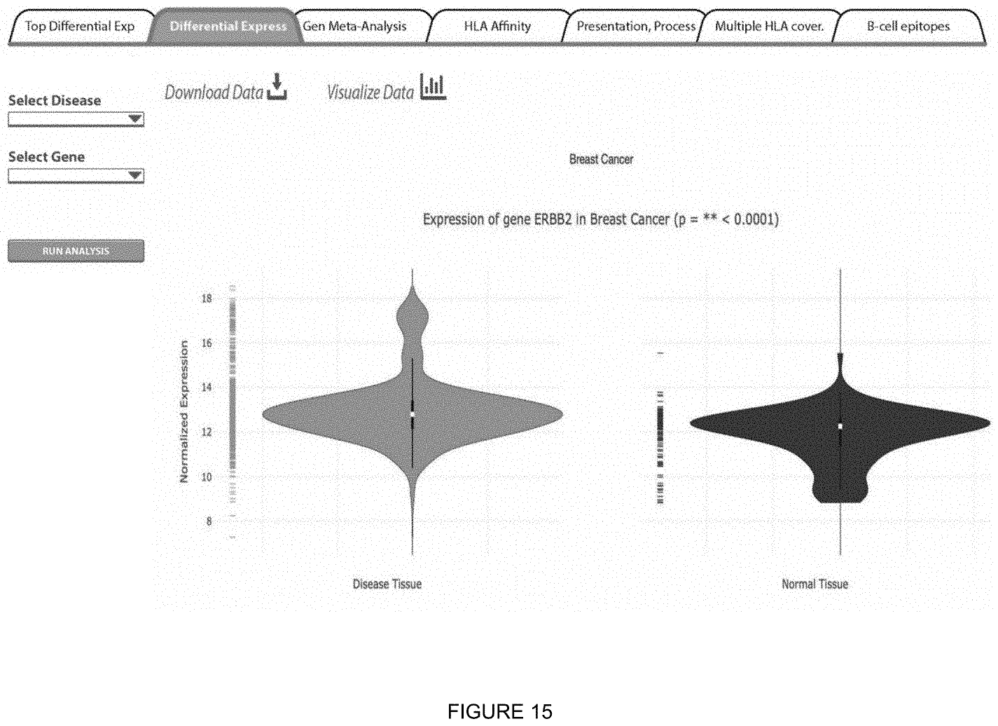

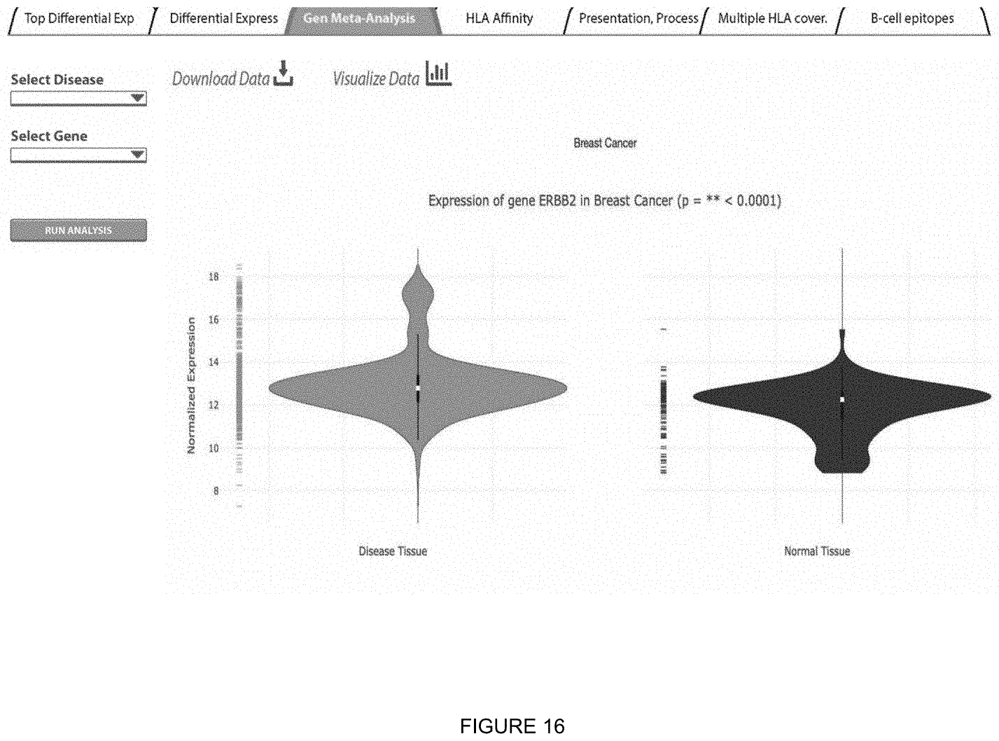

Non-limiting examples of DEM output are illustrated in FIG. 1 to FIG. 6C, FIG. 9 and FIG. 12 to FIG. 16. A non-limiting example of a unique amino acid subsequence identified in a disease-associated variant by a DEM includes an amino acid subsequence of SEQ ID NO:3, SEQ ID NO:19, SEQ ID NO:32 or SEQ ID NO:49.

Sequence Acquisition Interface (SAI)

Each of the MAAM, MCFM, MFLM and BEM modules can receive an input amino acid sequence from a sequence acquisition interface (SAD. In certain instances, a gene identifier (e.g., gene name, a gene tag (i.e., an abbreviated version of a gene name), accession number) can be inputted into a SAI, and an algorithm associated with the SAI can identify the gene identifier in a pre-compiled database and retrieve the associated amino acid sequence from the database. An amino acid sequence can be retrieved by an algorithm associated with an SAI from a NCO Protein database (e.g., https://www.ncbi.nlm.nih.gov/protein), for example. In certain instances, an amino acid sequence can be directly inputted into the SAI (e.g., copying and pasting an amino acid sequence). An input amino acid sequence can be a polypeptide amino acid sequence or a peptide amino acid sequence (e.g., a polypeptide or a peptide encoded by a gene or by a mRNA), or a portion of a polypeptide amino acid sequence or a peptide amino acid sequence, for example.

MHC Allele Affinity Determination Module (MAAM)

The methods described herein may include an analysis of binding affinity of a peptide to a major histocompatibility complex (MHC) molecule. A MHC molecule is encoded by a MHC allele. In some embodiments, a method herein comprises predicting the binding affinity of a peptide to an MHC molecule encoded by an MCH allele. In some embodiments, a method herein comprises predicting the binding affinity of a peptide to a plurality of MHC molecules encoded by multiple MHC alleles. In some embodiments, a method herein comprises predicting the binding affinity of a peptide to a supertype of MHC molecule encoded by a MHC supertype. A MHC supertype is a collection of MHC alleles having shared binding properties. A MHC allele is a human leukocyte antigen (HLA) allele in humans, and a MHC supertype is a HLA supertype in humans. A HLA allele is contemplated herein when referring to a MHC allele, and a HLA supertype is contemplated herein when referring to a MHC supertype.

MHC/HLA molecules are part of the immune system and are encoded by genes located on chromosome 6. MHC genes generally encode cell surface molecules that present antigenic peptides to the immune system. On the cell surface, MHC molecules bind to peptides that have been exported from within the cell. If the immune system recognizes the peptides as foreign (such as viral or bacterial peptides), it responds by triggering the infected cell to self-destruct. Certain MHC genes encode cell surface molecules that present antigenic peptides to T-cell receptors (TCRs) on T cells. MHC molecules that present antigen are divided into two main classes: class I MHC molecules and class II MHC molecules.

Class I MHC molecules are present as transmembrane glycoproteins on the surface of all nucleated cells. Class I molecules are made up of an alpha heavy chain bound to a beta-2 microglobulin molecule. The heavy chain includes two peptide-binding domains, an immunoglobulin (Ig)-like domain, and a transmembrane region with a cytoplasmic tail. In humans, the heavy chain of the class I molecule is encoded by genes at HLA-A, HLA-B, and HLA-C loci. CD8+ T cells react with class I MHC molecules. These lymphocytes often have a cytotoxic function, and can recognize infected cell. Typically, as every nucleated cell expresses class I MHC molecules, all infected cells can act as antigen-presenting cells for CD8+ T cells.

Class II MHC molecules typically are present on professional antigen-presenting cells (e.g., B cells, macrophages, dendritic cells, Langerhans cells). Class II MHC molecules are made up of two polypeptide chains (alpha and beta), where each chain has a peptide-binding domain, an Ig-like domain, and a transmembrane region with a cytoplasmic tail. Both polypeptide chains are encoded by genes in the HLA-DP, -DQ, or -DR region of chromosome 6. T cells reactive to class II molecules express CD4 and are often referred to as helper cells.

Individual serologically defined molecules encoded by the class I and II gene loci in the HLA system (for humans) are given standard designations (e.g., HLA-A1, HLA-B5, HLA-C1, HLA-DR1). Alleles generally are named to identify the gene, followed by an asterisk, numbers representing the allele group (often corresponding to the serologic antigen encoded by that allele), a colon, and numbers representing the specific allele (e.g., A*02:01, DRB1*01:03, DQA1*01:02). In certain instances, additional numbers are added after a colon to identify allelic variants that encode identical proteins, and after another colon, other numbers are added to denote polymorphisms in introns or in 5′ or 3′ untranslated regions (e.g., A*02:101:01:02, DRB1*03:01:01:02).

A MHC Allele Affinity Determination Module (MAAM) can be utilized to predict binding affinity of a particular amino acid sequence for a single MHC allele, multiple MHC alleles or a MHC supertype. A MAAM includes an artificial neural network (ANN). An ANN often includes a convolutional neural network (CNN) that contains a plurality of virtual neurons (i.e., nodes) arranged in capsules. A capsule is a group of virtual neurons and the capsules are arranged in layers in a CNN. A CNN includes weight values and bias values for each MHC allele and for each supertype. Weight values and bias values are generated by training the CNN using (i) a particular training dataset containing peptide and binding affinity information, and (ii) a particular training process. Weight values and bias values for each MHC allele depend on the training dataset and the training process.

A MAAM can predict a binding affinity of a particular amino acid subsequence for a selected MHC allele or supertype by a process that includes: (i) processing an input amino acid sequence into amino acid subsequences of a particular length; (ii) encoding the amino acid subsequences into numerical strings; (iii) estimating a binding affinity for each of the amino acid subsequences for a particular MHC allele or supertype from the numerical strings according to bias values and weight values associated with MHC allele or supertype in the CNN, thereby computing binding affinities for the amino acid subsequences for the particular MHC allele or supertype.

A MAAM can receive an amino acid sequence via an associated interface, and the interface can be a SAI described herein. A MAAM can receive a MHC allele or supertype selection via an associated interface, which can be a SAI described herein. The interface may acquire a user-selected MHC allele, MHC alleles or MHC supertype from a pre-compiled list of MHC alleles and/or supertypes. A MHC supertype can be a MHC class I supertype or a MHC class II supertype. A MHC class I supertype can be a HLA class I supertype. A HLA class I supertype can be a HLA-A supertype or HLA-B supertype. A HLA-A supertype can be a A1, A2, A3, A24 or A26 supertype and a HLA-B supertype can be a B7, B8, B27, B39, B44, B58 or B62 supertype. Alleles are provided for each HLA-A supertype and HLA-B supertype in Sidney et al., BMC Immunol 9, 1 (2008), doi.org/10.1186/1471-2172-9-1, and non-limiting examples include HLA-A*01:01, HLA-A*02:02, HLA-A*02:05, HLA-A*02:07, HLA-A*02:12, HLA-A*02:50, HLA-A*11:01, HLA-A*24:02, HLA-A*25:01, HLA-A*26:02, HLA-A*30:02, HLA-A*66:01, HLA-A*68:02, HLA-B*07:02, HLA-B*08:01, HLA-B*51:01, HLA-B*18:01, HLA-B*27:05 HLA-B*39:01, HLA-B*40:01, HLA-B*58:01 and HLA-B*15:01. A MHC class II supertype can be a HLA class II supertype, and a HLA class II supertype can be a DR, DQ or DP supertype. A HLA-DR supertype can be a DR1, DR3, DR4, DR5 or DR9 supertype, or can be chosen from four supertypes: (DRB1*0401, DRB1*0405, DRB1*0802, DRB1*1101), (DRB3*0101, DRB3*0202), (DRB1*0301, DRB1*1302), and the fourth containing the remaining DR proteins. A HLA-DQ supertype can be a DQ1, DQ2, DQ3 supertype, or can be chosen from two supertypes: (DQB1*0301, DQB1*0302, DQB1*0401) and (DQB1*0201, DQB1*0501, DQB1*0602). A HLA-DP supertype can be a DPw1, DPw2, DPw4 or DPw6 supertype, or can be chosen from the supertype (DPB1*0101, DPB1*0201, DPB1*0401, DPB1*0402, DPB1*0501, and DPB1*1401). Alleles are provided for HLA class II supertypes in Wang et al., Immunoinformatics 1184: 309-317 (2014), doi: 10.1007/978-1-4939-1115-8_17, and non-limiting examples include DRB1*03:01, DRB1*07:01, DRB1*15:01, DRB1*01:01, DRB1*04:01, DRB1*04:04, DRB1*04:05, DRB1*08:02 and DRB1*09:01.

A MAAM can process an input amino acid sequence into smaller subsequences. A MAAM can break down an input amino acid sequence into all possible consecutive amino acid subsequences of selected length “n.” In certain instances, “n” is an integer between 4 and 20 (e.g., 4, 5, 6, 7, 8, 9, 10, 11, 12, 13, 14, 15, 16, 17, 18, 19 or 20). For example, for the amino acid sequence MPPLLAPLLAPL (SEQ ID NO: 93), the possible amino acid sequences for n=9 are: MPPLLAPLL (SEQ ID NO: 94), PPLLAPLLA (SEQ ID NO: 95), PLLAPLLAP (SEQ ID NO: 96), and LLAPLLAPL (SEQ ID NO: 97). A MAAM can receive a value for “n” selected by a user via an interface (e.g., the interface utilized by a user to select the MHC allele or supertype, and/or enter or access the amino acid sequence). Output of the subsequence generating process can be a plurality of amino acid subsequences of length n for a corresponding input amino acid sequence.

A MAAM then can encode each of the amino acid subsequences. Any encoding process suitable for CNN processing can be utilized. A non-limiting example of an encoding process is integer coding followed by one-hot coding. In certain embodiments, each amino acid sequence is encoded with a number that depends on the amino acid composition, where each of the 20 amino acids is assigned a fixed value. In a non-limiting example, a numerical value can be assigned to each amino acid in each amino acid subsequence, where the numerical value is the molecular weight of the amino acid approximated to the next integer (i.e., rounded molecular weight value). For the amino acid sequence MPPLLAPLLAPL (SEQ ID NO: 93) (as an illustrative example), the rounded molecular weights are: M=149, P=115, P=115, L=131, L=131, A=089, P=115, L=131, L=131, A=089, P=115 and L=131. Encoding of the rounded molecular weight values results in the following string: 49115115131131089115131131089115131, which can be converted to a binary string: 111001011011111110001010110101111001100010101100100100000111101111001110011011 101111111010011110010111101011111111011. In another non-limiting example, a numerical value can be assigned to each amino acid in each amino acid subsequence, where the numerical value is an alphabetically assigned numerical value (i.e., each of the 20 amino acids is assigned a numerical value based on occurrence in the alphabet). For the amino acid sequence MPPLLAPLLAPL (SEQ ID NO: 93) (as an illustrative example), the alphabetically assigned numerical values are: M=13, P=15, P=15, L=11, L=11, A=01, P=15, L=11, L=11, A=01, P=15 and L=11. Encoding of the alphabetically assigned numerical values can result in the following string: 131515111101151111011511, which can be converted into a binary string: 11011110110010111001011001110001011011001001100110000001001111101110010110111.

Output from an encoding process can be an encoded string of values derived for each counterpart amino acid subsequence.

A CNN in a MAAM then can receive the encoded strings of values and generate binding affinity estimates for the amino acid subsequence counterparts according to weight values and bias values in the CNN specific for the selected MHC allele or supertype selected. Following is a non-limiting description of a process by which a CNN can generate MHC binding affinity estimates for the amino acid subsequences.

A capsule in a CNN is a group of virtual neurons with each neuron referred to as a node. Each virtual neuron is associated with a vector representing the instantiation parameters of a specific peptide. The length of the activity vector represents the probability that a peptide exists with its instantiation parameters, i.e. IC50 and its associated amino acid subsequence. The orientation of the activity vector is used to represent the instantiation parameters. Active capsules at one level make predictions, via transformation matrices, for the instantiation parameters of higher-level capsules. When multiple predictions agree, a higher level capsule becomes active. An iterative routing-by-agreement mechanism is in place, where a lower-level capsule prefers to send its output to higher level capsules. Activity vectors associated with higher level capsules have a relatively large scalar product when predictions of a higher level capsules are in agreement with predictions of lower-level capsules.

Within each node can be considered as a set of inputs, weight values, and bias values, and weight values and bias values can transform input as follows:

input->x(weight)+(bias)->output.

Within a CNN there often is an input layer, which can receive the input signals and pass them to the next layer. A CNN can contain a series of hidden layers that apply transformations to the input data. It is within the nodes of the hidden layers that the weights and biases can be applied. A final layer of a CNN can function as an output layer. An output layer often tunes the inputs from the hidden layers to produce the desired results (i.e., predictions).

Weight values and bias values are parameters inside a CNN that can be obtained by training. A trainable CNN randomizes the weight values and bias values before training initially begins. During training, both parameters are adjusted toward the desired values and the correct output. The two parameters differ to the extent of their influence upon the input data. Bias represents how far the predictions are from their intended value. Biases make up the difference between the actual output of a function and its intended output. A low bias value suggest that the network is making more assumptions about the form of the output, whereas a high bias value makes fewer assumptions about the form of the output. Weights, on the other hand, can be thought of as the strength of the connection. Weight affects the amount of influence a change in the input will have upon the output. A relatively low weight value will change input by a relatively small amount, whereas a larger weight value will more significantly change the input. Training generates weight values and bias values specific for each MHC allele in the CNN.

A CNN of a MAAM can be trained in any suitable manner. A training processes can implement supervised learning or non-supervised learning. A training process can include instructing a CNN to process a training dataset using multiple models. A model is a collection of operations that are followed until mean-squared error loss reaches a minimum value. In a non-limiting example, an ensemble of five models can be trained for each MHC allele, each on a randomly selected subset (80%) of the original data, with 20% used for validation. Models can be trained using an optimization algorithm (i.e., optimizer) that minimizes an output error rate. Non-limiting examples of optimization algorithms include Adaptive Moment Estimation (Adam) and Stochastic Gradient Descent (SGD). Metrics for determining efficacy of an optimizer include: speed of convergence (i.e., process of reaching a global optimum) and generalization (the model's performance on new data). Adam is efficient for convergence as it is an adaptive learning rate optimization algorithm that can be used to update network weights iteratively based on training data. The technique can update each parameter of a model, observe how a change would affect the objective function, choose a direction that would lower the error rate, and continue iterating until the objective function converges to the minimum. SGD often produces the same performance as Adam when the learning rate is slow, but Adam often outperforms SGD when dealing with complex predictions. In certain instances, training does not include generating a substitution matrix (e.g., a PAM or BLOSUM substitution matrix is not generated). Any suitable dataset can be utilized for training, and non-limiting examples of training sets are available at http://tools.iedb.org/mhci/download/. Data containing numeric affinity values (e.g., IC50 values) generally are selected for training input, and affinity values sometimes are transformed to a normalized value (e.g., via a logarithmic transformation). For example, a logo transformation can be implemented, transforming an illustrative measured IC50 value of 49.83 nanomolar in the training set to a value of 1.69.

A single MHC allele (e.g., HLA allele) can be selected, and a MAAM can estimate a binding affinity for each of the amino acid subsequences of length n for the selected allele based on weight values and bias values in a CNN for the selected allele. Multiple MHC alleles (e.g., HLA alleles) can be selected, and a MAAM can estimate an affinity for each of the amino acid subsequences of length n for each selected allele based on weight values and bias values in a CNN for each selected allele. A MHC supertype (e.g., an HLA supertype) can be selected, and a MAAM can estimate a binding affinity for each of the amino acid subsequences of length n for the selected supertype based on weight values and bias values in a CNN for the supertype.

Binding affinity values estimated by a CNN can be expressed as a molar binding affinity (e.g., a nanomolar binding affinity), for example. A binding affinity value can be expressed as a transformed value, such as a logarithmically transformed value of a molar binding affinity (e.g., a logarithmically transformed value of a nanomolar binding affinity). A non-limiting example of a logarithmic transformation of a molar binding affinity value is according to (1−log₅₀₀₀(affinity)). A binding affinity sometimes is expressed as an IC50 value. An IC50 value generally is 50% of the concentration of a peptide needed to completely compete a standard peptide out of the binding pocket of an MHC molecule. Generally, a relatively lower binding affinity value expressed in molar (e.g., nanomolar) units is indicative of a stronger binding affinity between a MHC molecule and a peptide, and similarly, a relatively lower IC50 value is indicative of a stronger binding affinity between a MHC molecule and a peptide. A binding affinity value estimated by a MAAM often is an IC50 binding affinity value expressed in molar units (e.g., nanomolar units).

A MAAM can assign an offset value for each of the amino acid subsequences. An offset value often is a position of the first amino acid in the amino acid subsequence with respect to the position it occurs in the longer amino acid sequence that contains the subject amino acid subsequence. For example, if an amino acid subsequence starts with “A” and that “A” residue is the second position in the longer amino acid sequence (e.g., the longer amino acid sequence begins with MA . . . ), the offset of the amino acid subsequence is assigned an offset value of 2.

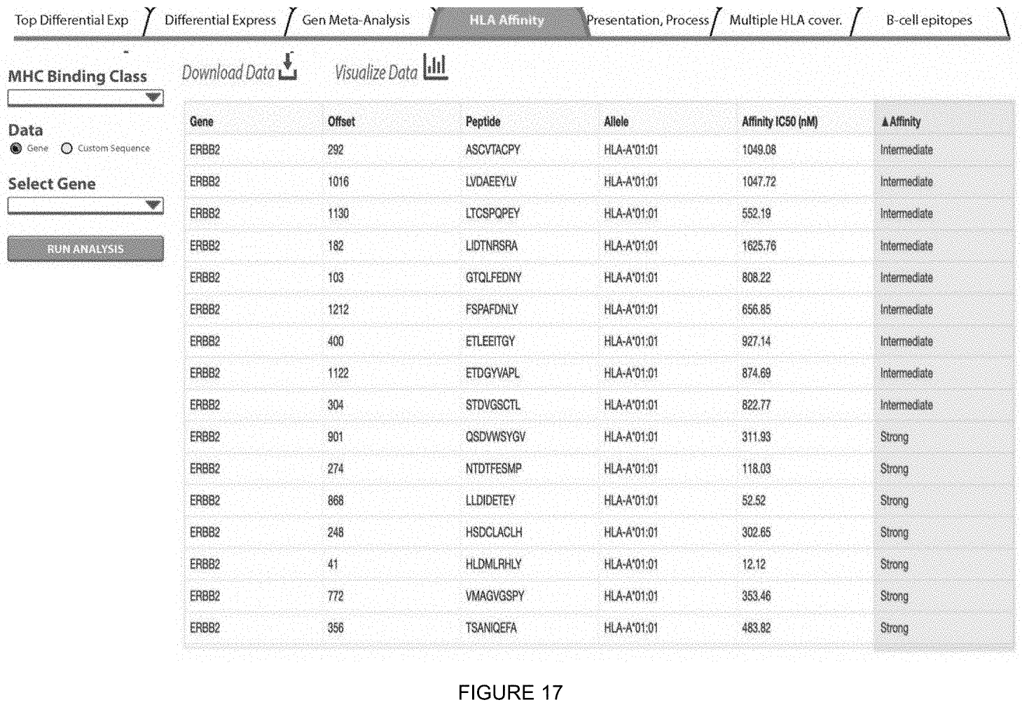

A MAAM can output a list that associates each amino acid subsequence with a binding affinity value determined according to a selected MHC allele or supertype. A MAAM can apply a binding affinity threshold and output a list containing only amino acid subsequences associated with a binding affinity less than, less-than-or-equal-to, greater than, or greater-than-or-equal-to the threshold. When a binding affinity threshold is expressed as molar binding affinity (e.g., nanomolar binding affinity), amino acid subsequences associated with a binding affinity value less than, or less-than-or-equal-to, the threshold can be selected for the outputted list. A list outputted by a MAAM can include, for each of the amino acid subsequences listed, two or more of: MHC allele designation or MHC supertype designation, associated MHC binding affinity value, normalized MHC binding affinity value, gene identifier associated with the longer amino acid sequence (e.g., gene name), offset value, index value, and subjective binding affinity descriptor (e.g., strong, intermediate). A subjective binding descriptor can be assigned according to a binding affinity value threshold. In certain instances, a “strong” subjective binding affinity descriptor can be assigned for an amino acid subsequence associated with a binding affinity that is less than 500 nM, an “intermediate” subjective binding affinity descriptor can be assigned for an amino acid subsequence associated with a binding affinity that is between 500 nanomolar and 1,000 nanomolar, and a “weak” subjective binding affinity descriptor can be assigned for an amino acid subsequence associated with a binding affinity that is greater than 1,000 nanomolar. A list outputted by a MAAM can be sorted, filtered and/or ranked according to any of the values or descriptors listed (e.g., MHC binding affinity value, MHC allele, MHC supertype). A non-limiting example of MAAM output is illustrated in FIG. 17.

It has been determined that a MAAM described herein can compute MHC binding affinity value estimations with less error than a commonly-utilized ANN referred to as NetMHC4.8 (http://www.cbs.dtu.dk/services/NetMHC/). A panel of 42 randomly selected peptides (i.e., amino acid subsequences) with experimentally measured IC50 values across 8 different HLA alleles (i.e., HLA-A*01:01, HLA-A*11:01, HLA-A*24:02, HLA-A*30:02, HLA-A*68:02, HLA-B*15:01, HLA-B*27:05, HLA-B*58:01) was utilized to determine an error proportion associated with HLA allele binding affinities (i.e., IC50) estimated by a MAAM described herein and estimated by NetMHC. Experimentally measured IC50 values ranged from 1 nM to 15,211 nM, with a median of 139 nM. The error proportion was calculated for each peptide/HLA allele pair as:

Error Proportion=(ABS(predicted IC50−measured IC50))/measured IC50

where ABS is the absolute value (without a sign). On average, the MAAM estimated binding affinity values with an error proportion 2.46 times lower than the error proportion for NetMHC estimations. For peptides having a measured IC50 greater than 6 nM, the MAAM consistently outperformed NetMHC, with the MAAM estimating binding affinities with an error proportion from 1.7 to 2.5 times lower than the error proportion for NetMHC estimations.

MHC Composite Feature Module (MCFM)

A MCFM can receive via an associated interface (i) a MHC allele, group of MHC alleles, or MHC supertype selection, and (ii) a length n value (e.g., a default value sometimes is n=9 for MHC class I predictions, and a default value sometimes is n=15 for MHC class II predictions). A MCFM also can receive via an associated interface an amino acid sequence (e.g., via a SAI described herein).

A MCFM also can independently receive and/or independently compute: (i) a proteasome cleavage score and/or (ii) a transporter affinity score. A proteasome cleavage score can be a value based on a likelihood of proteasome processing an input amino acid sequence into a particular amino acid subsequence. A proteasome cleavage score can be generated by executing a prediction process performed by NetChop3.0 described in Nielsen et al., Immunogenetics 57(1-2):33-41 (2005) doi: 10.1007/s00251-005-0781-7. A transporter affinity score can be a value based on a likelihood of a particular amino acid subsequence binding a transporter protein. A transporter affinity score can be a value based on a likelihood of a particular amino acid subsequence binding to a transporter associated with antigen processing (TAP), which is referred to as a TAP affinity score. A TAP affinity score can be calculated according to equation 3 in Peters et al., J Immunol 171 (4) 1741-1749 (2003) DOI: World Wide Web Uniform Resource Locator doi.org/10.4049/jimmunol.171.4.1741. An interface associated with a MCFM can send inputs with a request to a server to generate a proteasome cleavage score and a TAP affinity score, and a request may be sent to the NetCTL server (http://tools.iedb.org/netchop/) to generate these scores.

A MCFM can independently receive and/or independently compute a binding affinity value (i.e., IC50 binding affinity in nanomolar units) predicted for each MHC allele and/or each MHC supertype selected. A binding affinity value generated by a suitable process may be received by a MCFM. A MCFM can receive from a MAAM a binding affinity value predicted for each MHC allele and/or each MHC supertype selected. A MCFM can receive a binding affinity value from a NetCTL server (http://tools.iedb.org/netchop/). In certain instances a MCFM receives a binding affinity value in molar units (e.g., nanomolar units), and the binding affinity value is transformed into a normalized binding affinity value. A binding affinity value in molar units (e.g., nanomolar units) can be transformed into a normalized binding affinity value by a logarithmic transformation (e.g., a logarithmic transformation according to (1−log₅₀₀₀(affinity))).

A MCFM can receive or generate a composite score, for each MHC allele and/or MHC supertype selected, according to (i) a normalized MHC allele or MHC supertype binding affinity, (ii) a proteasome cleavage score, and (iii) a TAP affinity score. A composite score can be a weighted sum of (i), (ii) and (iii), and a weight equal to 1, 0.1 and 0.05 can be assigned to (i), (ii) and (iii) respectively. A composite score can be computed according to the following equation: composite score=(normalized binding affinity value)+((0.1)*(proteasome cleavage score)) ((0.05)*(TAP affinity score)), where the normalized binding affinity value can be a binding affinity value normalized by a percentile score (e.g., a first percentile score). A MFLM can generate an offset value for each of the amino acid subsequences.

A MCFM can output a list that can include, for each of the amino acid subsequences listed, two or more of: MHC allele designation or MHC supertype designation, associated composite score, associated MHC binding affinity value, normalized binding affinity value, proteasome cleavage score, transporter affinity score (e.g., TAP affinity score), gene identifier associated with the longer amino acid sequence (e.g., gene name), offset value, index value, and subjective binding affinity descriptor (e.g., strong, intermediate). A list outputted by a MCFM can be sorted, filtered and/or ranked according to any of the values or descriptors listed (e.g., composite score, MHC binding affinity value, MHC allele, MHC supertype). A MCFM can output a list for the variants according to the composite score, from highest composite score to lowest composite score, for example.

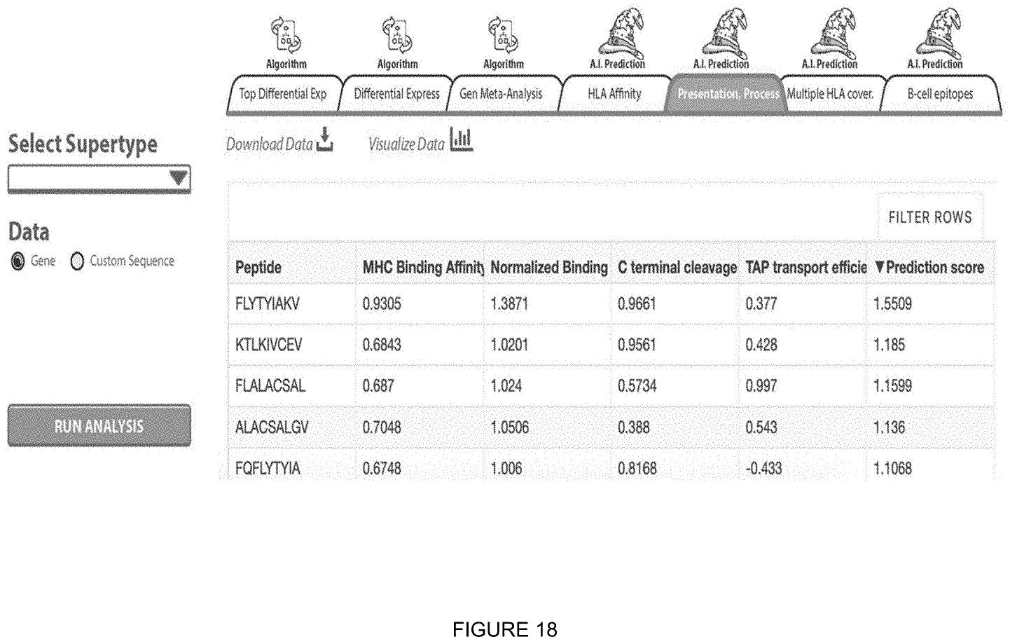

A MCFM can output a graphic representation of the longer amino acid sequence and mapped amino acid subsequences associated with a binding affinity value less than or less-than-or-equal to a threshold. If there are one or more regions of the longer amino acid sequence to which there are two or more amino acid subsequences co-mapped, a graphic representation can include a color rendition for the regions in which there are overlapping amino acid subsequences mapped that is different than the color rendition for regions in which only one amino acid subsequence is mapped or to which no amino subsequences are mapped. A region in which there are two overlapping amino acid subsequences mapped often has a color rendition in a graphic representation that is different than the color rendition of a region in which there are three overlapping amino acid subsequences, for example. Such a graphic representation is referred to as a “heat map” herein. A graphic representation (e.g., heat map) outputted by a MCFM can be associated with a MHC binding affinity value for each amino acid subsequence mapped to the longer amino acid sequence. In certain instances, a user can visualize a binding affinity value for each amino acid subsequence mapped to the longer amino acid subsequence in a graphic representation outputted by a MCFM (e.g., a user may visualize an associated binding affinity value estimate by “hovering” over a particular amino acid subsequence). A non-limiting example of MCFM output is illustrated in FIG. 18.

MHC Fragment Locator Module (MFLM)

A MFLM can facilitate identification and selection of amino acid subsequences for which a stronger MHC allele or MHC supertype binding affinity value have been estimated (e.g., for development of an immunotherapy). A MFLM can facilitate identification of a sub-region in a longer amino acid sequence to which multiple amino acid subsequences associated with stronger MHC binding affinity have been mapped. Such a sub-region, which can contain an amino acid subsequence longer than the mapped amino acid subsequences assessed for MHC binding, can facilitate development of an immunotherapy.

A MFLM can receive an amino acid subsequence and an accompanying MHC binding affinity value estimated by a MAAM for the amino acid subsequence. A MFLM can map the amino acid subsequence to a longer amino acid sequence counterpart. A longer amino acid sequence counterpart can be received by a MFLM, and can be received from a SAI. An amino acid subsequence can be mapped to a longer amino acid sequence from which it was derived, for example. Mapping can be performed by a MFLM by identifying a subsequence within the longer amino acid sequence having an exact match to an amino acid subsequence for which a MHC binding affinity value has been estimated. A MFLM can generate an offset value for each of the mapped amino acid subsequences.

The amino acid subsequences mapped can be limited to particular amino acid subsequences having MHC binding affinity estimates less than, or less-than-or-equal-to, a particular binding affinity threshold for one or more MHC alleles or MHC supertypes, when the binding affinity value is expressed in molar units (e.g., nanomolar units), for example. A MFLM can output a graphic representation of the longer amino acid sequence and mapped amino acid subsequences.

If there are one or more regions of the longer amino acid sequence to which there are two or more amino acid subsequences co-mapped, a graphic representation can include a color rendition for the regions in which there are overlapping amino acid subsequences mapped that is different than the color rendition for regions in which only one amino acid subsequence is mapped or to which no amino subsequences are mapped. A region in which there are two overlapping amino acid subsequences mapped often has a color rendition in a graphic representation that is different than the color rendition of a region in which there are three overlapping amino acid subsequences, for example. Such a graphic representation is referred to as a “heat map” herein.

A graphic representation (e.g., heat map) outputted by a MFLM can be associated with a MHC binding affinity value for each amino acid subsequence mapped to the longer amino acid sequence. In certain instances, a user can visualize a binding affinity value for each amino acid subsequence mapped to the longer amino acid subsequence in a graphic representation outputted by a MFLM (e.g., a user may visualize an associated binding affinity value estimate by “hovering” over a particular amino acid subsequence).

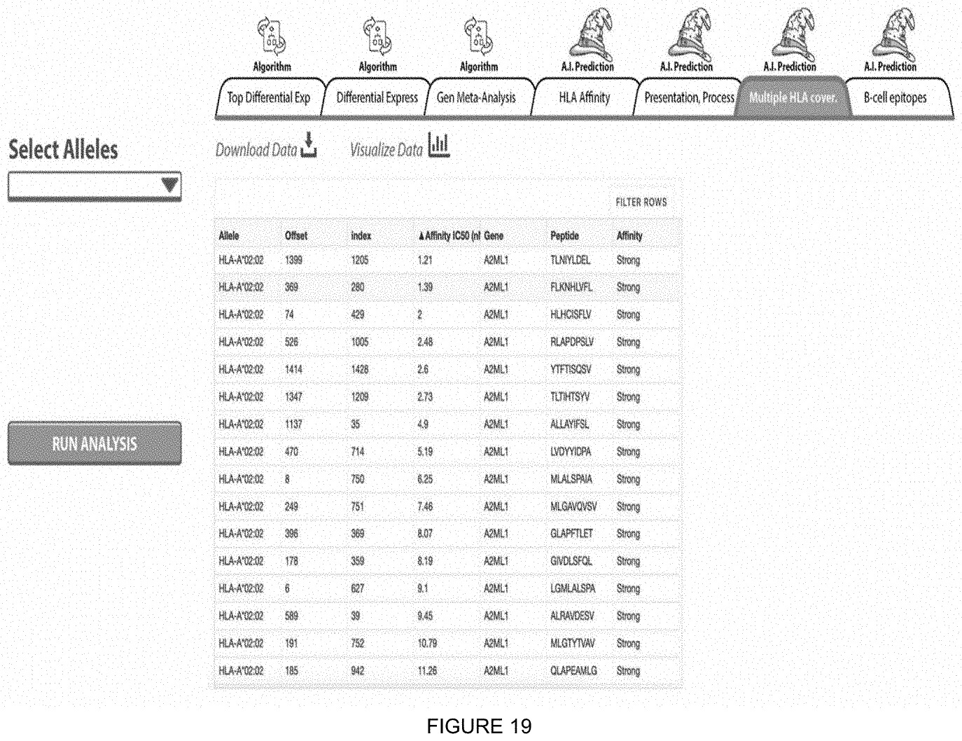

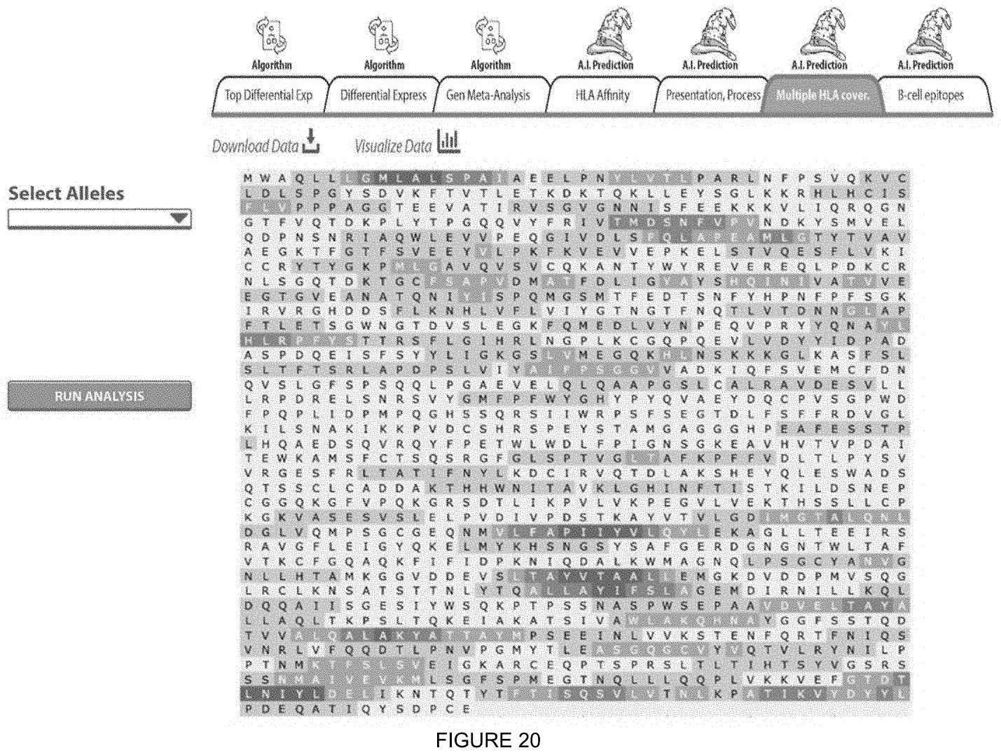

A MFLM can output a list in addition to a graphic representation. A list can include for each of the amino acid subsequences mapped to the longer amino acid sequence in the graphic representation, two or more of: MHC allele designation or MHC supertype designation, associated binding affinity value, normalized binding affinity value, gene identifier associated with the longer amino acid sequence (e.g., gene name), offset value, index value, and subjective binding affinity descriptor (e.g., strong, intermediate). A list outputted by a MFLM can be sorted, filtered and/or ranked according to any of the values or descriptors listed (e.g., MHC binding affinity value, MHC allele, MHC supertype). Non-limiting examples of MFLM output are illustrated in FIG. 19 and FIG. 20.

T-Cell Receptor Immunogenicity Determination Module (TIM)

The methods described herein may include an analysis of T-cell receptor (TCR) binding and/or activation. T cell receptors (TCRs) are immune proteins that specifically bind to antigenic molecules. TCRs are composed of two different polypeptides that are on the surface of T cells. They recognize, or specifically bind to, antigens bound to major histocompatibility complex (MHC) molecules. Typically, a TCR binds to an antigen, and the T cell is activated. By recognize is meant, for example, that the T cell receptor, or fragment or fragments thereof, such as TCR-alpha polypeptide and TCR-beta together, is capable of contacting the antigen and identifying it as a target. TCRs may comprise alpha and beta polypeptides, or chains. The alpha and beta polypeptides include two extracellular domains, a variable domain and a constant domain. The variable domain of the alpha and beta polypeptides has three complementarity determining regions (CDRs); CDR3 is generally considered the main CDR responsible for recognizing the epitope. The alpha polypeptide includes the V and J regions, generated by VJ recombination, and the beta polypeptide includes the V, D, and J regions, generated by VDJ recombination. The intersection of the VJ regions and VDJ regions corresponds to the CDR3 region.

For a peptide to be considered immunogenic, two conditions must be met: a) it should be presented on the surface of cells in conjunction with the appropriate HLA molecule and b) the peptide should bind to and activate the T-cell receptor (TCR) expressed on CD8+ T cells. The Immunotherapy Builder System (IBS) described herein addresses both of these requirements. Despite peptides being predicted to efficiently bind the certain HLA molecules, with high binding affinity (<500 nM), frequently they fail to demonstrate features of robust TCR binding. Therefore, despite the high probability of peptides being presented on the surface of antigen presenting cells, many false positives are likely detected, which are then incapable of engaging and activating the TCR. In order to improve the positive predictive value of peptide prioritization methods based on their immunogenicity, a score for TCR-engagement likelihood is needed, and it is provided by a T-Cell Receptor Immunogenicity Determination Module (TIM) described below.

A TIM can compute a T-cell receptor (TCR) interaction assessment score (hereafter an “immunogenicity score”) for each of one or more amino acid subsequences. A TIM can receive one or amino acid subsequences, which sometimes are identified by output of a MAAM, MCFM or MFLM. Such sequences can be inputted into a TIM. In certain instances, input amino acid subsequences received by a TIM can be amino acid subsequences associated with a strong and/or intermediate binding affinity value predicted by a MAAM, MCFM or MFLM. An amino acid subsequence is referred to as a “target peptide” for a TIM. A TIM can compute an immunogenicity score for each of the target peptides.

An immunogenicity score is a quantitative metric of a probability of functional T-cell recognition of a target peptide, and facilitates robust prioritization of target peptides with high expected immunogenicity. An immunogenicity score is computed according to (i) TCR-peptide contact potential profiling (CPP), a sequence-based simulation framework designed to mimic molecular recognition of MHC-presented peptides by TCR repertoire molecules, and (ii) one or more of the following features: target peptide length, amino acid in each position of a target peptide, and target peptide descriptors (i.e., sequence-based estimates of physicochemical properties).

A TIM can include a plurality of TCR fragment sequences in memory, or can access a database pre-populated with the TCR fragment sequences. A TCR includes an alpha-chain and a beta-chain and three complementarity-determining regions (CDRs; CDR1, CDR2 and CDR3) on each of the alpha-chain and the beta-chain. Each TCR fragment sequence in the plurality of TCR fragment sequences can be a member of a plurality of TCR CDR3 beta-chain fragment sequences. A TCR CDR3 beta-chain fragment sequence can be a portion of an amino acid subsequence of a TCR attributed to a CDR3 of a beta-chain of a TCR, or a portion thereof. In certain instances, the plurality of TCR CDR3 beta-chain fragment sequences includes (i) sliding window-generated fragment sequences, and/or (ii) portions of reverse-oriented TCR CDR3 beta-chain sequences, and/or (iii) TCR CDR3 beta-chain fragment sequences from CD4+ T-cells, and/or (iv) TCR CDR3 beta-chain fragment sequences from CD8+ T-cells.

A TIM can perform a CPP process that includes (i) generating a plurality of target fragment sequences from the amino acid sequences of target peptides received into the TIM, (ii) generating an alignment pair between each target fragment sequence and a TCR fragment sequence, and (iii) computing an alignment score for each alignment pair. A TIM can generate an optimized alignment between the target fragment sequence and the TCR fragment sequence in each alignment pair, where the optimized alignment maximizes the alignment score for the alignment pair. Computing the alignment score can include generating a sum of pairwise scores, where each of the pairwise scores is a score generated for an amino acid in the target fragment sequence and an aligned amino acid in the TCR fragment sequence for the alignment pair. In certain instances, computing an alignment score can include (i) implementing amino acid pairwise contact potential (AACP) scales from the AAIndex database (http://www.genome.jp/aaindex/AAindex/list_of_potentials); and/or (ii) implementing the pairwiseAlignment function in the Biostrings package in Bioconductor (https://www.bioconductor.org/). In certain instances, there are no gaps between the target fragment sequence and the TCR fragment sequence in each alignment pair. In certain instances, the target fragment sequence and/or the TCR fragment sequence in each alignment pair is about 3 amino acids to about 8 amino acids in length for MHC class I polypeptide fragment sequences and/or about 3 amino acids to about 11 amino acids in length for MHC class II polypeptide fragment sequences. Performing a CPP can include assembling the alignment scores into amino acid pairwise contact potential matrices.

A TIM can compute an immunogenicity score for each input target peptide according to output of the CPP. In certain instances, a TIM can compute an immunogenicity score for each input target peptide according to amino acid pairwise contact potential matrices from the CPP. In certain instances, a TIM can compute an immunogenicity score for each input target peptide according to amino acid pairwise contact potential matrices from the CPP and one or more features chosen from: target peptide length, amino acid in each position of a target peptide, and target peptide descriptors peptide descriptor features and MHC binding prediction features. A non-limiting example of an algorithm for generating an immunogenicity score using CPP is REpitope described in Ogishi et al., Frontiers in Immunology 10: 827 (2019) doi: 10.3389/fimmu.2019.00827.

A TIM can output a list of target peptide sequences (i.e., the amino acid subsequences that have been received by the TIM (e.g., inputted into the TIM)) and associated immunogenicity values, and the list can be sorted, filtered and/or ranked according to immunogenicity values. In certain instances, a disease-associated immunogenic candidate polypeptide sequence is selected from among the target peptide sequences ranked in the top 20% according to immunogenicity score.

B-Cell Receptor Epitope Determination Module (BEM)

The methods described herein may include an analysis of B-cell receptor (BCR) binding and/or activation. The B-cell receptor (BCR) is made up of immunoglobulin molecules that form a type 1 transmembrane receptor protein typically located on the outer surface of B cells, and the BCR generally controls the activation of B cells. B cells bind antigens and undergo endocytosis and antigen presentation. A BCR's binding moiety generally includes a membrane-bound antibody that has a unique and randomly determined antigen-binding site. The BCR for an antigen is a sensor that is required for B cell activation, survival, and development. A B cell is activated by its first encounter with an antigen that binds to its receptor, the cell proliferates and differentiates to generate a population of antibody-secreting plasma B cells and memory B cells. A BCR generally has two functions upon interaction with the antigen: 1) signal transduction, involving changes in receptor oligomerization, and 2) mediate internalization for subsequent processing of the antigen and presentation of peptides to helper T cells.

A B-Cell Receptor Epitope Determination Module (BEM) can receive an input amino acid sequence and identify one or more B-cell receptor (BCR) epitopes in the amino acid sequence. An input amino acid sequence can be received by a BEM from a SAI.

A BEM can predict a location of one or more linear BCR epitopes, and optionally conformational BCR epitopes, in an input amino acid sequence using a combination of a hidden Markov model and a propensity scale process, or a Random Forest Regression (RF) algorithm. In certain instances, a BEM has been trained using a dataset containing epitopes annotated from antibody-antigen protein structures (e.g., AntiJen dataset (http://www.jenner.ac.uk/AntiJen)). A BEM can be trained by a process described in Larsen et al., Immunome Res. 2: 2 (2006) (BepiPred 1.0) or Jespersen et al., Nucleic Acids Research 45 (2017) doi: 10.1093/nar/gkx346 (BepiPred 2.0). A BEM can generate a score for each amino acid in an input amino acid sequence indicative of the probability that the amino acid exists within a BCR epitope. Amino acids within an input amino acid sequence having a score above a threshold can be predicted as being within a BCR epitope.

A threshold can be configurable (e.g., in an interface), and a default value for a threshold sometimes is 0.35 or 0.5. Values of the scores often are not affected by the threshold. A BEM may include sensitivity and specificity values, computed on the basis of epitope/non-epitope predictions, for different thresholds. A BEM can display such sensitivity and specificity values for informing a threshold selection. For example, increasing a threshold value can result in fewer epitopes displayed and shorter displayed epitopes.

A BEM sometimes computes one or more scores pertaining to likelihood that an amino acid in an input sequence exists in a secondary structure in the corresponding polypeptide. A score can be generated for likelihood an amino acid is in an alpha helix structure and a score can be generated for likelihood an amino acid is in a beta sheet structure. A BEM also can compute a score pertaining to likelihood that an amino acid is exposed or buried in the corresponding polypeptide. Scores pertaining to secondary structure and exposure can be generated using any suitable process (e.g., using NetSurfP; Petersen et al., BMC Struct. Biol. 9, 51 (2009)).

A BEM can output a graphic representation showing the input amino acid sequence annotated with (i) a score above a threshold displayed adjacent to each corresponding amino acid in the input sequence; and/or (ii) an indicator as to whether an amino acid in the input sequence is located in a BCR epitope (e.g., an “E” indicator). A graphic representation output by a BEM can include one or more scores and/or one or more color gradients scaled to (i) the score for each amino acid in the input sequence (e.g., a first amino acid having a score higher than a second amino acid can be annotated with a different color) and/or (ii) likelihood that an amino acid exists in a secondary structure (e.g., a color gradient scaled to likelihood an amino acid in the input sequence exists in an alpha helix secondary structure; a color gradient scaled to likelihood an amino acid in the input sequence exists in a beta sheet secondary structure); and/or (iii) likelihood that an amino acid is buried or exposed in the corresponding polypeptide.

Immunotherapy Builder System (IBS) implementation

An IBS or one or more portions thereof may be implemented with any type of hardware and/or software, and may be a pre-programmed general purpose computing device. For example, the system may be implemented using a server, a personal computer, a portable computer, a thin client, or any suitable device or devices. The system and/or components thereof may be a single device at a single location, or multiple devices at a single, or multiple, locations that are connected together using any appropriate communication protocols over any communication medium such as electric cable, fiber optic cable, or in a wireless manner.

An IBS is illustrated and discussed herein as having a plurality of modules that perform particular functions. Modules are illustrated based on their function for clarity purposes only, and do not necessarily represent specific hardware or software. Modules may be hardware and/or software implemented to substantially perform the particular functions discussed. Modules may be combined together, or divided into additional modules based on the particular function desired. Thus, the disclosure should not be construed to limit the technology described, but merely be understood to illustrate an example of implementation.

An IBS or one or more portions thereof can include clients and servers. A client and server generally are remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

An IBS or one or more portions thereof can include a back-end component (e.g., a data server), and/or a middleware component (e.g., an application server), and/or a front-end component. A front-end component can be a client computer having a graphical user interface or a Web browser through which a user can interact with an implementation described herein. Components of an IBS can be interconnected by any form or medium of digital data communication, e.g., a communication network. Examples of communication networks include a local area network (“LAN”) and a wide area network (“WAN”), an inter network (e.g., the Internet), and peer-to-peer networks (e.g., ad hoc peer-to-peer networks).

An IBS or one or more portions thereof can be implemented in digital electronic circuitry, or in computer software, firmware, or hardware, including the structures disclosed in this specification and their structural equivalents, or in combinations of one or more of them. An IBS or one or more portions thereof can be implemented as one or more computer programs, i.e., one or more modules of computer program instructions, encoded on computer storage medium for execution by, or to control the operation of, data processing apparatus. Alternatively or in addition, program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. A computer storage medium can also be, or be included in, one or more separate physical components or media (e.g., multiple CDs, disks, or other storage devices).

Operations performed by an IBS or one or more portions thereof can be implemented as operations performed by a “data processing apparatus” on data stored on one or more computer-readable storage devices or received from other sources. A “data processing apparatus” encompasses different types of apparatus, devices, and machines for processing data, non-limiting examples of which include a programmable processor, a computer, a system on a chip, or multiples of, or combinations, of the foregoing. An apparatus can include special purpose logic circuitry, e.g., an FPGA (field programmable gate array) or an ASIC (application specific integrated circuit). An apparatus can also include, in addition to hardware, code that creates an execution environment for a computer program, e.g., code that constitutes processor firmware, a protocol stack, a database management system, an operating system, a cross-platform runtime environment, a virtual machine, or a combination of one or more of them. An apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

Processes and logic flows described for an IBS or one or more portions thereof can be performed by one or more programmable processors executing one or more computer programs to perform actions by operating on input data and generating output. Processes and logic flows can be performed by, and apparatus can also be implemented as, special purpose logic circuitry, e.g., an FPGA (field programmable gate array) or an ASIC (application-specific integrated circuit).

Processors suitable for the execution of a computer program include general and special purpose microprocessors, for example, and any one or more processors of any kind of digital computer. Generally, a processor receives instructions and data from a read-only memory or a random access memory or both. Essential elements of a computer are a processor for performing actions in accordance with instructions and one or more memory devices for storing instructions and data. A computer can also include, or be operatively coupled to receive data from or transfer data to (or both), one or more mass storage devices for storing data, e.g., magnetic, magneto-optical disks, or optical disks. A computer need not include devices. A computer can be embedded in a device including but not limited to a mobile telephone, a personal digital assistant (PDA), a mobile audio or video player, a Global Positioning System (GPS) receiver, or a portable storage device (e.g., a universal serial bus (USB) flash drive). Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. A processor and memory can be supplemented by, or incorporated in, special purpose logic circuitry.

An IBS can include one or more of the following modules: Differential Expression Module (DEM), MHC Allele Affinity Determination Module (MAAM), a MHC Fragment Locator Module (MFLM), a MHC Composite Feature Module (MCFM), a T-Cell Receptor Immunogenicity Determination Module (TIM), a B-Cell Receptor Epitope Determination Module (BEM). An IBS optionally can include a Sequence Acquisition Interface (SAI). In certain instances, an IBS can include a MAAM; a MAAM and a MFLM; a MAAM and a DEM; or a MAAM, a MFLM and a DEM.

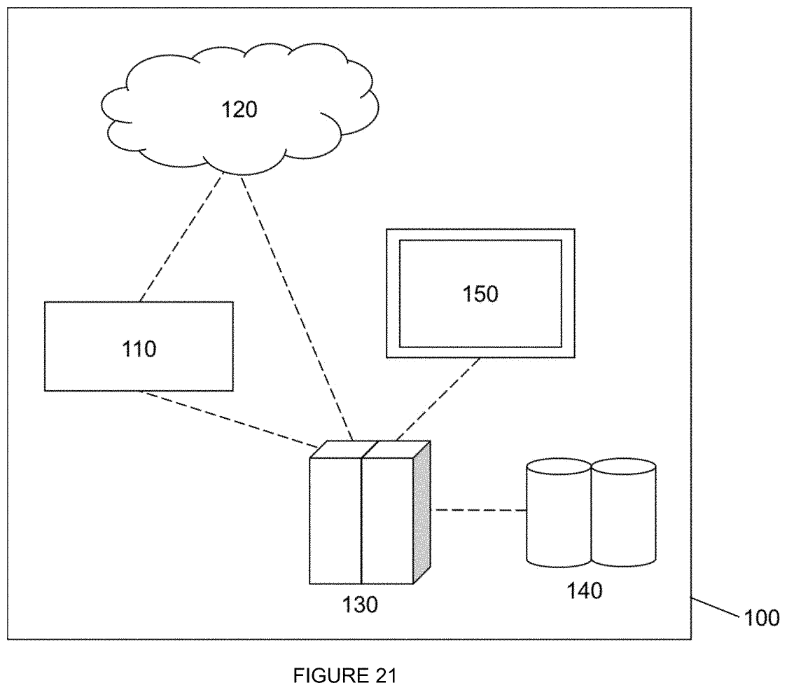

A non-limiting IBS implementation is illustrated in FIG. 21. In FIG. 21, illustrated is an IBS implementation (100) that includes an interface (110; e.g., a Sequence Acquisition Interface (SAI)), a network (120), one or more servers (130), memory (140) and a display (150). In certain instances, network (120) can be within one or more servers (130). Certain modules can be co-localized on a single server, and certain modules can be located on two or more separate servers. In certain implementations, a MAAM, a MAAM and a MFLM, a MAAM and a DEM, or a MAAM and a MFLM and a DEM, exist on the same server or the same group of servers. In certain implementations, memory (140) includes one or more databases. For example, amino acid sequences and associated expression levels can be stored in a database in memory (140). In certain implementations, memory (140) can include weight values and bias values for implementation in a MAAM. Output for one or more modules described herein can depicted on display (150).

In certain instances, a (i) DEM is implemented, outputting a plurality of disease-associated variants, and (ii) amino acid sequences for the variants are received by one or more of a MAAM, a MFLM, a MCFM, a TIM and a BEM, where output (e.g., list of amino acid subsequences and/or graphical representation) of the MAAM, MFLM, MCFM, TIM and/or BEM identifies immunogenic portions (i.e., amino acid subsequences; peptides containing the amino acid subsequences) of the disease-associated variants identified by the DEM. In certain instances, a DEM is not implemented, one or more amino acid sequences are inputted into one or more of a MAAM, a MFLM, a MCFM, a TIM and a BEM, where output (e.g., list of amino acid subsequences and/or graphical representation) of the MAAM, MFLM, MCFM, TIM and/or BEM identifies immunogenic portions (i.e., amino acid subsequences; peptides containing the amino acid subsequences) of the one or more input amino acid sequences.

An immunogenic portion (i.e., amino acid subsequence; peptide containing the amino acid subsequence) can be an immunotherapy candidate, and can be identified by (i) operation of a threshold, and/or (ii) observation by a user. In certain instances, an amino acid subsequence (i.e., peptide containing the amino acid subsequences) can be identified by output of a MAAM, a MCFM and/or a MFLM (e.g., list of amino acid subsequences and/or graphical representation). For example, amino acid subsequences having an estimated MHC allele or MHC supertype binding affinity value (e.g., molar affinity value) less than, or less-than-or-equal-to, a binding affinity threshold, and can be identified as an immunotherapy candidate. Such amino acid subsequences, or longer amino acid sequences each containing one or more of the amino acid subsequences, can be utilized as input for another immunologic assessment process (e.g., input for a TIM and/or input for a BEM).

In certain instances, an amino acid subsequence (i.e., peptide containing the amino acid subsequence) that contains (i) an amino acid subsequence of length n identified by a MAAM, a MCFM and/or a MFLM as having an estimated MHC allele or MHC supertype binding affinity value (e.g., molar binding affinity value) less than or less-than-or-equal-to a threshold, and (ii) a flanking N-terminal amino acid subsequence and/or a flanking C-terminal amino acid subsequence present in a longer amino acid sequence that contains the subject amino acid subsequence of length n, can be identified as an immunotherapy candidate. A flanking N-terminal amino acid subsequence or a flanking C-terminal amino acid subsequence, sometimes is identified according to presence in other overlapping amino acid subsequences of length n identified as having strong or intermediate binding affinity to the same MHC allele or other MHC alleles or a MHC supertype (e.g., identified when amino acid subsequences are mapped to a longer amino acid sequence that contains them (e.g., by a MCFM or MFLM). An amino acid sequence comprising (i) an amino acid subsequence of length n, and (ii) a flanking N-terminal amino acid subsequence and/or a flanking C-terminal amino acid subsequence present in a longer amino acid sequence that contains the subject amino acid subsequence of length n, can be output of a MAAM, a MCFM and/or a MFLM (e.g., list of amino acid subsequences and/or graphical representation), and often output of a MCFM or a MFLM (e.g., graphical representation). Such amino acid subsequences, or longer amino acid sequences each containing one or more of the amino acid subsequences, can be utilized as input for another immunologic assessment process (e.g., input for a TIM and/or input for a BEM).

Output of a MAAM, a MCFM and/or a MFLM can narrow a group of potential immunogenic peptides. In certain instances, peptides identified by a MAAM, a MCFM and/or a MFLM as exhibiting strong or intermediate binding to a particular MHC allele, can be further assessed for strong or intermediate binding to one or more other MHC alleles and/or strong or intermediate binding to a MHC supertype. Peptides identified as exhibiting strong binding or intermediate binding to a plurality of MHC alleles and/or one or more MHC supertypes, can be selected as immunotherapy candidates. A heat map output by a MFLM and/or MCFM can facilitate identification of peptides having strong or intermediate binding to multiple MHC alleles. A plurality of such immunotherapy candidates can be further narrowed by another module, such as a TIM and/or BEM module, as described herein.

When two or three of a MAAM, a MCFM and a MFLM are implemented, output of each module can be assessed, and one subset containing amino acid subsequences and/or amino acid sequences that each include one or more of the amino acid subsequences can be prepared as a single output resulting from implementation of the MAAM/MCFM, MAAM/MFL, MCFM/MFLM or MAAM/MCFM/MFLM combination.

Output of a TIM can narrow a group of potential immunogenic peptides identified by another module. In certain instances, amino acid subsequences (i.e., peptides containing the amino acid subsequences) identified are output by a MAAM, a MCFM and/or a MFLM as having an estimated MHC allele or MHC supertype binding affinity value (e.g., molar binding affinity value) less than, or less-than-or-equal-to, a binding affinity threshold. Such amino acid subsequences, or longer amino acid sequences each containing one or more of the amino acid subsequences, can be received by a TIM, a subset of the amino acid subsequences output by the MAAM, a MCFM and/or a MFLM can be identified by the TIM as having an immunogenicity score above a threshold, and the amino acid subsequences or longer amino acid sequences in the subset can be selected as immunotherapy candidates.

Output of a BEM can narrow a group of potential immunogenic peptides identified by another module. In certain instances, amino acid subsequences (i.e., peptides containing the amino acid subsequences) can be identified by a BEM in amino acid sequences received by one or more of a MAAM, a MCFM, a MFLM and/or a TIM, and the immunogenic portions identified by the BEM can be compared for overlap with the immunogenic peptides identified by the MAAM, MCFM, MFLM and/or TIM. A subset of immunogenic peptides identified by a BEM that overlap (or portions of the peptides overlap) with immunogenic peptides identified by MAAM, MCFM, MFLM and/or TIM, can be selected as immunotherapy candidates.

One or more of a DEM, a MAAM, a MFLM, a MCFM, a TIM and a BEM can receive one or more amino acid sequences from a SAI. In certain instances, one or more of a MAAM, a MFLM, a MCFM and a BEM can receive one or more amino acid sequences from a SAI.

In certain instances, one or more of a MAAM, a MFLM and a MCFM receive one or more input amino acid sequences and are implemented, outputting a subset of amino acid subsequences in the input amino acid sequences according to a MHC binding affinity value threshold, where the amino acid subsequences in the subset, or longer amino acid sequences each containing one or more of the amino acid subsequences, can be considered immunogenic candidates. In certain instances, (i) one or more of a MAAM, a MFLM and a MCFM receive one or more input amino acid sequences and are implemented, outputting a subset of amino acid subsequences in the input amino acid sequences according to a MHC binding affinity value threshold; and (ii) amino acid subsequences in the subset, and/or longer amino acid sequences each containing one or more of the amino acid subsequences, is received by a TIM and the TIM is implemented, outputting a subset of the input amino acid subsequences and/or amino acid sequences according to an immunogenicity score threshold, where the amino acid subsequences in the subset outputted by the TIM, or longer amino acid sequences each containing one or more of the amino acid subsequences, can be considered immunogenic candidates. Optionally, a BEM is implemented, where the BEM receives the same one or more input amino acid sequences received by one or more of a MAAM, a MFLM and a MCFM, outputting a first subset of amino acid subsequences according to a BCR score threshold, where a second subset of the amino acid subsequences in the first subset, or longer amino acid sequences each containing one or more of the amino acid subsequences in the first subset, that also are present in the output of one or more of the MAAM, MFLM, MCFM and TIM, can be considered immunogenic candidates. In certain instances, a DEM is implemented, outputting one or more disease-associated amino acid sequence variants, which are received by one or more of a MAAM, a MFLM and a MCFM as one or more input amino acid sequences in one or more of the foregoing implementations.

In certain instances, a DEM is implemented, outputting one or more disease-associated amino acid sequence variants, which are received by BEM as one or more input amino acid sequences.

Characterization of amino acid subsequences identified by IBS

Amino acid subsequences identified by an IBS can be considered immunogenic peptide candidates, which can be characterized using a variety of methods. Non-limiting examples of such methods include proteomic analyses and immunogenic analyses. A non-limiting example of an immunogenic peptide candidate includes a peptide comprising an amino acid sequence of SEQ ID NO:4, SEQ ID NO:20 or SEQ ID NO:27.

In certain instances, a proteomic analysis can include determining presence, absence or amount of a peptide candidate in polypeptides of disease samples. In a non-limiting example, presence, absence or amount of a peptide candidate is determined using a mass spectrometry assessment of polypeptides of disease samples.

In certain instances, an immunogenic analysis can include measuring binding affinity of a peptide candidate, or portion thereof, to a MHC complex (e.g., HLA complex). In certain instances, an immunogenic analysis can include determining presence, absence or amount of a peptide candidate, or portion thereof, presented on the surface of an antigen presenting cell (APC), such as a dendritic cell, for example. In certain instances, an immunogenic analysis can include determining presence, absence or amount of T-cell receptor (TCR) binding to a peptide candidate/MHC complex (or complex containing a portion of a peptide candidate and MHC molecules). In certain instances, an immunogenic analysis can include determining presence, absence or amount of T-cell cytotoxicity in a system that includes a peptide candidate, an APC and a T-cell. In certain instances, an immunogenic analysis can include determining presence, absence or amount of a peptide candidate, or portion thereof, bound to a B-cell receptor. In certain instances, an immunogenic analysis can include determining presence, absence or amount of an interaction of a peptide candidate, or portion thereof, with one or more of invariant natural killer T cells (iNKT), NK cells, and mucosal-associated innate T (MAIT) cells.

Use of Amino Acid Subsequences and Amino Acid Sequences Identified by an IBS

IBS outputs (e.g., amino acid subsequences, amino acid sequences) may be utilized in a variety of applications. For example, certain applications may include an immunotherapeutic strategy targeting tumor-associated isoforms. In one embodiment, the immunotherapeutic strategy targeting tumor-associated isoforms is a peptide vaccine. In another embodiment, the peptide is encoded by a DNA vector. In another embodiment, the immunotherapeutic strategy targeting tumor-associated isoforms is an adoptive T-cells therapy. In another embodiment, the immunotherapeutic strategy targeting tumor-associated isoforms is monoclonal antibody therapy. In one embodiment, the immunotherapeutic strategy targeting tumor-specific mutations is a peptide vaccine. In another embodiment, the peptide is encoded by a DNA vector. In another embodiment, the immunotherapeutic strategy targeting tumor-specific mutations is an adoptive T-cells therapy. In another embodiment, the immunotherapeutic strategy targeting tumor-specific mutations is monoclonal antibody therapy.

Amino acid subsequences identified by an IBS can be considered immunogenic peptide candidates, which can be utilized in a variety of applications. A non-limiting example of an immunogenic peptide candidate includes a peptide comprising an amino acid sequence of SEQ ID NO:4, SEQ ID NO:20 or SEQ ID NO:27. In certain instances, an immunogenic peptide identified by an IBS can be synthesized. A peptide can be synthesized using any suitable method, including by chemical synthesis, by in vitro translation, or by recombinant translation in host cells. Thus, provided herein is a composition comprising a peptide identified by an IBS and a method for synthesizing a peptide identified by an IBS.

A synthesized peptide can be combined with one or more suitable pharmaceutically acceptable adjuvants and/or one or more suitable pharmaceutically acceptable carriers suitable for a vaccine. Non-limiting examples of pharmaceutically acceptable vaccine adjuvants include aluminum (e.g., amorphous aluminum hydroxyphosphate sulfate (AAHS), aluminum hydroxide, aluminum phosphate, potassium aluminum sulfate); monophosphoryl lipid A (MPL) and aluminum salt (AS04); oil in water emulsion composed of squalene (MF59); monophosphoryl lipid A (MPL) and QS-21, a natural compound extracted from the Chilean soapbark tree, combined in a liposomal formulation (AS01B); and cytosine phosphoguanine (CpG 1018). A pharmaceutically acceptable carrier can be a diluent, excipient, or vehicle included in a composition containing the peptide that is administered. A pharmaceutically acceptable carrier can be a sterile liquid. A pharmaceutically acceptable aqueous carrier, such as a saline solution, aqueous dextrose solution and/or glycerol solution can be included when a vaccine is administered intravenously. A vaccine composition that includes an immunogenic peptide identified by an IBS and a pharmaceutically acceptable adjuvant, and optionally includes a pharmaceutically acceptable carrier, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A polynucleotide encoding an immunogenic peptide identified by an IBS can be prepared. A polynucleotide can include one or more elements from a different type of organism from which the polynucleotide portion encoding the immunogenic peptide originated. In certain instances, a polynucleotide can include a polynucleotide portion form a human gene that encodes a peptide identified by an IBS, and can include one or more polynucleotide portions from a different organism (e.g., from a virus; from a bacterium). A polynucleotide sometimes is an expression vector or expression plasmid. A polynucleotide sometimes is a vector or plasmid suitable for administration to a subject, and can be formulated as a vaccine. A polynucleotide vector sometimes is a DNA vector (e.g., a DNA virus or based on a DNA virus (e.g., double-stranded DNA virus), including a herpesvirus, an adenovirus, and a poxvirus) or a RNA vector (e.g., RNA virus or based on a RNA virus, including a retrovirus and a ssRNA virus). Non-limiting examples of polynucleotide vectors are described in Deng et al., Vaccine 33(48): 6938-6946 (2015). A vaccine composition that includes a polynucleotide encoding an immunogenic peptide identified by an IBS, and optionally includes a pharmaceutically acceptable carrier and/or adjuvant, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A composition that includes an antigen presenting cell (APC) and a peptide identified by an IBS can be prepared. In certain implementations, a composition that includes an APC and a polynucleotide encoding a peptide identified by an IBS can be prepared. A composition that includes an APC transduced with a polynucleotide encoding a peptide identified by an IBS can be prepared. A polynucleotide encoding a peptide identified by an IBS sometimes is an expression plasmid or expression vector, and an APC can be transduced by the polynucleotide. An APC can be transduced by a polynucleotide in any suitable manner, non-limiting examples of which include transduction by naked polynucleotide and transduction by electroporation. A non-limiting example of an APC is a dendritic cell. In certain implementations, a composition comprising an APC and a peptide identified by an IBS (e.g., a vaccine composition), or a composition comprising an APC transduced with a polynucleotide encoding a peptide identified by an IBS (e.g., a vaccine composition), where the composition optionally includes a pharmaceutically acceptable carrier and/or adjuvant, can be administered to a subject in need thereof (e.g., human subject) in an amount sufficient to induce an immune response to the peptide in the subject. Such a composition can be administered to a subject as part of a method for treating a condition (e.g., a disease, a cancer) in which inducing an immune response against the peptide can treat the condition (e.g., ameliorate a symptom associated with the condition).

A peptide identified by an IBS can be administered to a subject for production of antibodies that immunospecifically bind to the peptide. Antibodies produced can be polyclonal antibodies or monoclonal antibodies, for example. A peptide identified by an IBS can be included in a composition administered to an animal subject (e.g., rabbit subject, camelid subject), antiserum can be obtained, and polyclonal antibodies optionally may be enriched and/or isolated from the antiserum. A peptide identified by an IBS can be included in a composition administered to an animal subject (e.g., murine subject, guinea pig subject, rabbit subject) and spleen cells from the subject can be combined with myeloma cells under conditions that produce monoclonal antibody generating hybridomas. Hybridomas can be screened for those that produce monoclonal antibodies that immunospecifically bind to the peptide administered to the animal subject. Accordingly, compositions containing the peptide can be administered to a subject as part of a method for manufacturing antibodies (e.g., monoclonal antibodies, polyclonal antibodies) that immunospecifically bind to the peptide.

Samples

Provided herein are methods for analyzing nucleic acid and/or polypeptides from a sample. Nucleic acid and/or polypeptides may be isolated from a sample obtained from a subject (e.g., a test subject). A subject can be any living or non-living organism, including but not limited to a human, a non-human animal, a plant, a bacterium, a fungus, a protest, or a pathogen. Any human or non-human animal can be selected, and may include, for example, mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female. A subject may be any age (e.g., an embryo, a fetus, an infant, a child, an adult). A subject may be a patient having a disease or condition, a patient suspected of having a disease or condition, a patient in remission for a disease or condition, a patient with a family history of a disease or condition, and/or a subject obtaining a screen for a disease or condition. A subject may be a cancer patient, a patient suspected of having cancer, a patient in remission, a patient with a family history of cancer, and/or a subject obtaining a cancer screen. A subject may be a patient having an infection or infectious disease or infected with a pathogen (e.g., bacteria, virus, fungus, protozoa, and the like), a patient suspected of having an infection or infectious disease or being infected with a pathogen, a patient recovering from an infection, infectious disease, or pathogenic infection, a patient with a history of infections, infectious disease, pathogenic infections, and/or a subject obtaining an infectious disease or pathogen screen.

A sample may be isolated or obtained from any type of suitable biological specimen or sample (e.g., a test sample). A nucleic acid sample may be isolated or obtained from a single cell, a plurality of cells (e.g., cultured cells), cell culture media, conditioned media, a tissue, an organ, or an organism (e.g., bacteria, yeast, or the like).

A sample or test sample may be any specimen that is isolated or obtained from a subject or part thereof (e.g., a human subject, a subject having a disease or condition, a cancer patient, a patient having an infection or infectious disease, a tumor, an infected organ or tissue, a diseased organ or tissue). Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., cancer biopsy), celocentesis sample, cells (blood cells, normal cells, abnormal cells (e.g., cancer cells)) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof.

In some embodiments, a sample is isolated or obtained from a specimen (e.g., healthy or diseased specimen), cell(s) (e.g., healthy or diseased cell), tissue (e.g., healthy or diseased tissue), organ (e.g., healthy or diseased organ), and/or the like of an animal (e.g., an animal subject). A sample obtained from a healthy specimen, cell, tissue, and/or organ may be referred to as a non-disease sample. A non-disease sample may be obtained from a subject with no diagnosis of a particular disease (e.g., cancer), no history of a particular disease (e.g., cancer), and/or no suspicion of having a particular disease (e.g., cancer). A sample obtained from a diseased specimen, cell, tissue, and/or organ may be referred to as a disease sample. A disease sample may be obtained from a subject with a diagnosis of a particular disease or condition (e.g., cancer), a history of a particular disease or condition (e.g., cancer), and/or a subject suspected of having a particular disease or condition (e.g., cancer).

A sample can be a liquid sample. Examples of liquid samples include, but are not limited to, blood or a blood product (e.g., serum, plasma, or the like), urine, cerebral spinal fluid, saliva, sputum, biopsy sample (e.g., liquid biopsy for the detection of cancer), a liquid sample described above, the like or combinations thereof. In certain embodiments, a sample is a liquid biopsy, which generally refers to an assessment of a liquid sample from a subject for the presence, absence, progression or remission of a disease (e.g., cancer). A liquid biopsy can be used in conjunction with, or as an alternative to, a sold biopsy (e.g., tumor biopsy).

A sample may be a tumor sample (i.e., a sample isolated from a tumor). The term “tumor” generally refers to neoplastic cell growth and proliferation, whether malignant or benign, and may include pre-cancerous and cancerous cells and tissues. The terms “cancer” and “cancerous” generally refer to the physiological condition in mammals that is typically characterized by unregulated cell growth/proliferation. Examples of cancer include, but are not limited to, carcinoma, lymphoma, blastoma, sarcoma, leukemia, squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, various types of head and neck cancer, and the like.

Nucleic Acid

Nucleic acid may be analyzed and/or prepared using the methods described herein. The terms nucleic acid(s), nucleic acid molecule(s), nucleic acid fragment(s), target nucleic acid(s), nucleic acid template(s), template nucleic acid(s), nucleic acid target(s), target nucleic acid(s), polynucleotide(s), polynucleotide fragment(s), target polynucleotide(s), polynucleotide target(s), polynucleotide sequence(s), and the like may be used interchangeably throughout the disclosure. The terms refer to nucleic acids of any composition, such as DNA (e.g., complementary DNA (cDNA; synthesized from any RNA or DNA of interest), genomic DNA (gDNA), genomic DNA fragments, mitochondrial DNA (mtDNA), recombinant DNA (e.g., plasmid DNA), and the like), RNA (e.g., message RNA (mRNA), short inhibitory RNA (siRNA), ribosomal RNA (rRNA), transfer RNA (tRNA), microRNA, transacting small interfering RNA (ta-siRNA), natural small interfering RNA (nat-siRNA), small nucleolar RNA (snoRNA), small nuclear RNA (snRNA), long non-coding RNA (lncRNA), non-coding RNA (ncRNA), transfer-messenger RNA (tmRNA), precursor messenger RNA (pre-mRNA), small Cajal body-specific RNA (scaRNA), piwi-interacting RNA (piRNA), endoribonuclease-prepared siRNA (esiRNA), small temporal RNA (stRNA), signal recognition RNA, telomere RNA, RNA highly expressed in a tumor, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, virus, bacterium, autonomously replicating sequence (ARS), mitochondria, centromere, artificial chromosome, chromosome, or other nucleic acid able to replicate or be replicated in vitro or in a host cell, a cell, a cell nucleus or cytoplasm of a cell in certain embodiments. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), alternative splice variants, and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense,” “plus” strand or “minus” strand, “forward” reading frame or “reverse” reading frame) and double-stranded polynucleotides. The term “gene” refers to a section of DNA involved in producing a polypeptide chain; and generally includes regions preceding and following the coding region (leader and trailer) involved in the transcription/translation of the gene product and the regulation of the transcription/translation, as well as intervening sequences (introns) between individual coding regions (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

Nucleic acid may be described herein as being complementary to another nucleic acid and/or being capable of hybridizing to another nucleic acid. The terms “complementary” or “complementarity” or “hybridization” generally refer to a nucleotide sequence that base-pairs by non-covalent bonds to a region of a nucleic acid. In the canonical Watson-Crick base pairing, adenine (A) forms a base pair with thymine (T), and guanine (G) pairs with cytosine (C) in DNA. In RNA, thymine (T) is replaced by uracil (U). As such, A is complementary to T and G is complementary to C. In RNA, A is complementary to U and vice versa. In a DNA-RNA duplex, A (in a DNA strand) is complementary to U (in an RNA strand). Typically, “complementary” or “complementarity” or “capable of hybridizing” refer to a nucleotide sequence that is at least partially complementary. These terms may also encompass duplexes that are fully complementary such that every nucleotide in one strand is complementary or hybridizes to every nucleotide in the other strand in corresponding positions.

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position.

Nucleic acid analyzed by the methods described herein may be from a category or collection of nucleic acids. For example, nucleic acid may from a genome, a transcriptome, a library (e.g., a DNA library (a genomic DNA library, a cDNA library), an RNA library (an mRNA library)), a nucleic acid pool, and the like or combinations thereof. A genome generally refers to a complete list of nucleotides (A, C, G, and T) that make up the chromosomes of an individual or a species, and includes both the genes (coding regions) and noncoding DNA, and may include mitochondrial DNA. A transcriptome generally refers to a set of RNA transcripts, including coding and non-coding, in an individual or a population of cells, and sometimes refers to all RNAs, or just mRNA, depending on the context. Data obtained from a transcriptome may be used to analyze processes such as cellular differentiation, carcinogenesis, transcription regulation, and biomarker discovery, for example. The transcriptome is related to other “omes” such as, for example, the proteome, metabolome, translatome, exome, meiome, and thanatotranscriptome, which describe specific types of RNA transcripts. A nucleic acid library generally refers to a plurality of polynucleotide molecules (e.g., a sample of nucleic acids) that are prepared, assembled and/or modified for a specific process, non-limiting examples of which include immobilization on a solid phase (e.g., a solid support, a flow cell, a bead), enrichment, amplification, cloning, detection and/or for nucleic acid sequencing. In certain embodiments, a nucleic acid library is prepared prior to or during a sequencing process. A nucleic acid library (e.g., sequencing library) can be prepared by a suitable method as known in the art. A nucleic acid library can be prepared by a targeted or a non-targeted preparation process.

Nucleic acid may be derived from one or more sources (e.g., biological sample, blood, cells, serum, plasma, buffy coat, urine, lymphatic fluid, skin, hair, soil, and the like) by methods known in the art. In some embodiments, a sample is collected from a subject and nucleic acid is extracted from the sample. Any suitable method can be used for isolating, extracting and/or purifying DNA from a biological sample, non-limiting examples of which include methods of DNA preparation (e.g., described by Sambrook and Russell, Molecular Cloning: A Laboratory Manual 3d ed., 2001), various commercially available reagents or kits, such as DNeasy®, RNeasy®, QlAprep®, QlAquick®, and QlAamp® (e.g., QlAamp® Circulating Nucleic Acid Kit, QiaAmp® DNA Mini Kit or QiaAmp® DNA Blood Mini Kit) nucleic acid isolation/purification kits by Qiagen, Inc. (Germantown, Md.); GenomicPrep™ Blood DNA Isolation Kit (Promega, Madison, Wis.); GFX™ Genomic Blood DNA Purification Kit (Amersham, Piscataway, N.J.); DNAzoI®, ChargeSwitch®, Purelink®, GeneCatcher® nucleic acid isolation/purification kits by Life Technologies, Inc. (Carlsbad, Calif.); NucleoMag®, NucleoSpin®, and NucleoBond® nucleic acid isolation/purification kits by Clontech Laboratories, Inc. (Mountain View, Calif.); the like or combinations thereof. In certain aspects, the nucleic acid is isolated from a fixed biological sample, e.g., formalin-fixed, paraffin-embedded (FFPE) tissue. Genomic DNA from FFPE tissue may be isolated using commercially available kits—such as the AllPrep® DNA/RNA FFPE kit by Qiagen, Inc. (Germantown, Md.), the RecoverAll® Total Nucleic Acid Isolation kit for FFPE by Life Technologies, Inc. (Carlsbad, Calif.), and the NucleoSpin® FFPE kits by Clontech Laboratories, Inc. (Mountain View, Calif.).

In some embodiments, nucleic acid is extracted from cells (e.g., tumor cells, healthy cells) using a cell lysis procedure. Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. In some instances, a high salt and/or an alkaline lysis procedure may be utilized. In some instances, a lysis procedure may include a lysis step with EDTA/Proteinase K, a binding buffer step with high amount of salts (e.g., guanidinium chloride (GuHCl), sodium acetate) and isopropanol, and binding DNA in this solution to silica-based column. In some instances, a lysis protocol includes certain procedures described in Dabney et al., Proceedings of the National Academy of Sciences 110, no. 39 (2013): 15758-15763.

Nucleic acid may be provided for conducting methods described herein with or without processing of the sample(s) containing the nucleic acid. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. In certain examples, cancer cell nucleic acid can be purified from a mixture comprising cancer cell and non-cancer cell nucleic acid. In certain examples, nucleosomes comprising small fragments of cancer cell nucleic acid can be purified from a mixture of larger nucleosome complexes comprising larger fragments of non-cancer nucleic acid. In some embodiments, nucleic acid is provided for conducting methods described herein without prior processing of the sample(s) containing the nucleic acid. For example, nucleic acid may be analyzed directly from a sample without prior extraction, purification, partial purification, and/or amplification.

Nucleic acids may be amplified under amplification conditions. The term “amplified” or “amplification” or “amplification conditions” as used herein refers to subjecting a target nucleic acid in a sample or a nucleic acid product generated by a method herein to a process that linearly or exponentially generates amplicon nucleic acids having the same or substantially the same nucleotide sequence as the target nucleic acid, or part thereof. In certain embodiments, the term “amplified” or “amplification” or “amplification conditions” refers to a method that comprises a polymerase chain reaction (PCR).

Polypeptides

Polypeptides may be analyzed and/or prepared using the methods described herein. A polypeptide generally refers to a polymer, linked by peptide bonds, that has a sequence of amino acids encoded by a polynucleotide. Proteins or portions thereof (e.g., a subunit of a protein) are generally made up of polypeptides. A peptide generally refers to a portion or fragment of a larger polypeptide. In some instances, a peptide refers to a polymer containing between about 2 amino acids to about 10 amino acids, 2 amino acids to about 20 amino acids, or about 2 amino acids to about 30 amino acids. Peptides, may include, for example, dipeptides, tripeptides, tetrapeptides, and oligopeptides. Amino acids that have been incorporated into peptides and/or polypeptides may be referred to as residues. Peptides and polypeptides typically have an N-terminal (amine group) residue at one end and C-terminal (carboxyl group) residue at the opposite end, and amino acid sequences are typically read in the N-terminal to C-terminal direction.

### EXAMPLES

The examples set forth below illustrate certain embodiments and do not limit the technology.

### Example 1: Identification of Hot-Spot Mutations and Tumor-Associated Splice Isoforms

Prediction of Hot-Spot Mutations (DEM Implementation)

The technology described herein can predict hot-spot mutations for a given cancer type by accessing the TOGA mRNA-level data and ranking non-synonymous mutations based on the percent frequency with which they are detected in a given cancer diagnosis.

Examples of hot-spot mutations identified by the technology described herein are provided in the tables below. Mutations are annotated in the following format: wt—amino acid position—mutated amino acid. So for example, R273C means that “R” at position 273 is mutated into a “C.”

Ovarian cancer

| Gene | Percent | |
|---|---|---|
| (accession no.) | cases | Mutation |
| TP53 (AAA59987.1) | 94 | R273C/R273H |
| BRACA1 (P38398) | 3 | C47W/L1216 frameshift |
| NF1 (P21359) | 4 | L550P/L1726R |

Pancreatic cancer

| Gene | Percent | |
|---|---|---|
| (accession no.) | cases | Mutation |
| PIK3CA (P42336) | 14 | E545K/H1047R |
| PTEN (P60484) | 7 | R130G/K128N |
| KRAS (P01116) | 7 | G12C/G12V |

Breast cancer

| Gene | Percent | |
|---|---|---|
| (accession no.) | cases | Mutation |
| PIK3CA (P42336) | 31 | E545G/H1047L |
| GATA3 (P23771) | 9 | S408 frameshift |
| MAP3K1 (Q13233) | 6 | W201R/W1433S |

Colon cancer

| Gene | Percent | |
|---|---|---|
| (accession no.) | cases | Mutation |
| APC (P25054) | 76 | K1292T/R2431K/D2519G/E136K |
| KRAS (P01116) | 43 | G12D/G12V/G12F |
| FBXW7 (Q969H0) | 16 | R465H/R505C |

AML

| Gene | Percent | |
|---|---|---|
| (accession no.) | cases | Mutation |
| DNMT3A (Q9Y6K1) | 30 | R882H |

BCL

| Gene | Percent | |
|---|---|---|
| (accession no.) | cases | Mutation |
| CREBBP (Q92793) | 30 | R1446H |

MM

| Gene | Percent | |
|---|---|---|
| (accession no.) | cases | Mutation |
| KRAS (P01116) | 25 | G12V |

CLL

| Gene | Percent | |
|---|---|---|
| (accession no.) | cases | Mutation |
| SF3B1 (O75533) | 15 | K700E |

Non-mutated amino acid sequences for the genes listed in the tables above are as follows:

TP53 (GENBANK accession no. AAA59987.1)
(SEQ ID NO: 33)
MEEPQSDPSVEPPLSQETFSDLWKLLPENNVLSPLPSQAMDDLMLSPDDIEQWFTEDPGPDEAPRMPEAAPRVAPGPAAP
TPAAPAPAPSWPLSSSVPSQKTYQGSYGERLGELHSGTAKSVTCTYSPALNKMFCQLAKTCPVQLWVDSTPPPGTRVRAM
AIYKQSQHMTEVVRRCPHHERCSDSDGLAPPQHLIRVEGNLRVEYLDDRNTFRHSVVVPYEPPEVGSDCTTIHYNYMCNS
SCMGGMNRRPILTIITLEDSSGNLLGRNSFEVRVCACPGRDRRTEEENLRKKGEPHHELPPGSTKRALPNNTSSSPQPKK
KPLDGEYFTLQIRGRERFEMFRELNEALELKDAQAGKEPGGSRAHSSHLKSKKGQSTSRHKKLMFKTEGPDSD
BRACA1 (UNIPROT accession no. P38398)
(SEQ ID NO: 34)
MDLSALRVEEVQNVINAMQKILECPICLELIKEPVSTKCDHIFCKFCMLKLLNQKKGPSQCPLCKNDITKRSLQESTRFS
QLVEELLKIICAFQLDTGLEYANSYNFAKKENNSPEHLKDEVSIIQSMGYRNRAKRLLQSEPENPSLQETSLSVQLSNLG
TVRTLRTKQRIQPQKTSVYIELGSDSSEDTVNKATYCSVGDQELLQITPQGTRDEISLDSAKKAACEFSETDVTNTEHHQ
PSNNDLNTTEKRAAERHPEKYQGSSVSNLHVEPCGTNTHASSLQHENSSLLLTKDRMNVEKAEFCNKSKQPGLARSQHNR
WAGSKETCNDRRTPSTEKKVDLNADPLCERKEWNKQKLPCSENPRDTEDVPWITLNSSIQKVNEWFSRSDELLGSDDSHD
GESESNAKVADVLDVLNEVDEYSGSSEKIDLLASDPHEALICKSERVHSKSVESNIEDKIFGKTYRKKASLPNLSHVTEN
LIIGAFVTEPQIIQERPLTNKLKRKRRPTSGLHPEDFIKKADLAVQKTPEMINQGTNQTEQNGQVMNITNSGHENKTKGD
SIQNEKNPNPIESLEKESAFKTKAEPISSSISNMELELNIHNSKAPKKNRLRRKSSTRHIHALELVVSRNLSPPNCTELQ
IDSCSSSEEIKKKKYNQMPVRHSRNLQLMEGKEPATGAKKSNKPNEQTSKRHDSDTFPELKLTNAPGSFTKCSNTSELKE
FVNPSLPREEKEEKLETVKVSNNAEDPKDLMLSGERVLQTERSVESSSISLVPGTDYGTQESISLLEVSTLGKAKTEPNK
CVSQCAAFENPKGLIHGCSKDNRNDTEGFKYPLGHEVNHSRETSIEMEESELDAQYLQNTEKVSKRQSFAPFSNPGNAEE
ECATESAHSGSLKKQSPKVTFECEQKEENQGKNESNIKPVQTVNITAGFPVVGQKDKPVDNAKCSIKGGSRFCLSSQFRG
NETGLITPNKHGLLQNPYRIPPLFPIKSFVKTKCKKNLLEENFEEHSMSPEREMGNENIPSTVSTISRNNIRENVFKEAS
SSNINEVGSSTNEVGSSINEIGSSDENIQAELGRNRGPKLNAMLRLGVLQPEVYKQSLPGSNCKHPEIKKQEYEEVVQTV
NTDFSPYLISDNLEQPMGSSHASQVCSETPDDLLDDGEIKEDTSFAENDIKESSAVFSKSVQKGELSRSPSPFTHTHLAQ
GYRRGAKKLESSEENLSSEDEELPCFQHLLFGKVNNIPSQSTRHSTVATECLSKNTEENLLSLKNSLNDCSNQVILAKAS
QEHHLSEETKCSASLFSSQCSELEDLTANTNTQDPFLIGSSKQMRHQSESQGVGLSDKELVSDDEERGTGLEENNQEEQS
MDSNLGEAASGCESETSVSEDCSGLSSQSDILTTQQRDTMQHNLIKLQQEMAELEAVLEQHGSQPSNSYPSIISDSSALE
DLRNPEQSTSEKAVLTSQKSSEYPISQNPEGLSADKFEVSADSSTSKNKEPGVERSSPSKCPSLDDRWYMHSCSGSLQNR
NYPSQEELIKVVDVEEQQLEESGPHDLTETSYLPRQDLEGTPYLESGISLFSDDPESDPSEDRAPESARVGNIPSSTSAL
KVPQLKVAESAQSPAAAHTTDTAGYNAMEESVSREKPELTASTERVNKRMSMVVSGLTPEEFMLVYKFARKHHITLTNLI
TEETTHVVMKTDAEFVCERTLKYFLGIAGGKWVVSYFWVTQSIKERKMLNEHDFEVRGDVVNGRNHQGPKRARESQDRKI
FRGLEICCYGPFTNMPTDQLEWMVQLCGASVVKELSSFTLGTGVHPIVVVQPDAWTEDNGFHAIGQMCEAPVVTREWVLD
SVALYQCQELDTYLIPQIPHSHY
NF1 (UNIPROT accession no. P21359)
(SEQ ID NO: 35)
MAAHRPVEWVQAVVSREDEQLPIKTGQQNTHTKVSTEHNKECLINISKYKFSLVISGLTTILKNVNNMRIFGEAAEKNLY
LSQLIILDTLEKCLAGQPKDTMRLDETMLVKQLLPEICHFLHTCREGNQHAAELRNSASGVLFSLSCNNFNAVFSRISTR
LQELTVCSEDNVDVHDIELLQYINVDCAKLKRLLKETAFKFKALKKVAQLAVINSLEKAFWNWVENYPDEFTKLYQIPQT
DMAECAEKLFDLVDGFAESTKRKAAVWPLQIILLILCPETIQDISKDVVDENNMNKKLFLDSLRKALAGHGGSRQLTESA
AIACVKLCKASTYINWEDNSVIFLLVQSMVVDLKNLLFNPSKPFSRGSQPADVDLMIDCLVSCFRISPHNNQHFKICLAQ
NSPSTEHYVLVNSLHRIITNSALDWWPKIDAVYCHSVELRNMFGETLHKAVQGCGAHPAIRMAPSLTEKEKVTSLKEKEK
PTDLETRSYKYLLLSMVKLIHADPKLLLCNPRKQGPETQGSTAELITGLVQLVPQSHMPEIAQEAMEALLVLHQLDSIDL
WNPDAPVETFWEISSQMLFYICKKLTSHQMLSSTEILKWLREILICRNKFLLKNKQADRSSCHFLLFYGVGCDIPSSGNT
SQMSMDHEELLRTPGASLRKGKGNSSMDSAAGCSGTPPICRQAQTKLEVALYMFLWNPDTEAVLVAMSCFRHLCEEADIR
CGVDEVSVHNLLPNYNTEMEFASVSNMMSTGRAALQKRVMALLRRIEHPTAGNTEAWEDTHAKWEQATKLILNYPKAKME
DGQAAESLHKTIVKRRMSHVSGGGSIDLSDTDSLQEWINMTGFLCALGGVCLQQRSNSGLATYSPPMGPVSERKGSMISV
MSSEGNADTPVSKFMDRLLSLMVCNHEKVGLQIRTNVKDLVGLELSPALYPMLFNKLKNTISKFFDSQGQVLLTDTNTQF
VEQTIAIMKNLLDNHTEGSSEHLGQASIETMMLNLVRYVRVLGNMVHAIQIKTKLCQLVEVMMARRDDLSFCQEMKFRNK
MVEYLTDWVMGTSNQAADDDVKCLTRDLDQASMEAVVSLLAGLPLQPEEGDGVELMEAKSQLFLKYFTLFMNLLNDCSEV
EDESAQTGGRKRGMSRRLASLRHCTVLAMSNLLNANVDSGLMHSIGLGYHKDLQTRATFMEVLTKILQQGTEFDTLAETV
LADRFERLVELVTMMGDQGELPIAMALANVVPCSQWDELARVLVTLFDSRHLLYQLLWNMFSKEVELADSMQTLFRGNSL
ASKIMTFCFKVYGATYLQKLLDPLLRIVITSSDWQHVSFEVDPTRLEPSESLEENQRNLLQMTEKFFHAIISSSSEFPPQ
LRSVCHCLYQATCHSLLNKATVKEKKENKKSVVSQRFPQNSIGAVGSAMFLRFINPAIVSPYEAGILDKKPPPRIERGLK
LMSKILQSIANHVLETKEEHMRPENDFVKSNEDAARRFFLDIASDCPTSDAVNHSLSFISDGNVLALHRLLWNNQEKIGQ
YLSSNRDHKAVGRRPFDKMAILLAYLGPPEHKPVADTHWSSLNLISSKFEEFMTRHQVHEKEEFKALKILSIFYQAGISK
AGNPIFYYVARRFKIGQINGDLLIYHVLLTLKPYYAKPYEIVVDLTHIGPSNRFKIDFLSKWFVVFPGFAYDNVSAVYIY
NCNSWVREYTKYHERLLTGLKGSKRLVFIDCPGKLAEHIEHEQQKLPAATLALEEDLKVFHNALKLAHKDTKVSIKVGST
AVQVISAERTKVLGQSVFLNDIYYASEIEEICLVDENQFTLTIANQGTPLIFMHQECEAIVQSIIHIRTRWELSQPDSIP
QHTKIRPKDVPGILLNIALLNLGSSDPSLRSAAYNLLCALICTFNLKIEGQLLETSGLCIPANNTLFIVSISKTLAANEP
HLTLEFLEECISGFSKSSIELKHLCLEYMTPWLSNLVRFCKHNDDAKRQRVTAILDKLITMTINEKQMYPSIQAKIWGSL
GQIIDLLDVVLDSFIKTSAIGGLGSIKAEVMADTAVALASGNVKLVSSKVIGRMCKIIDKICLSPIPTLEQHLMWDDIAI
LARYMLMLSFNNSLDVAAHLPYLFHVVTFLVATGPLSLRASTHGLVINIIHSLCICSQLHFSEETKQVLRLSLIEFSLPK
FYLLFGISKVKSAAVIAFRSSYRDRSFSPGSYERETFALTSLETVTEALLEIMEACMRDIPTCKWLDQWTELAQRFAFQY
NPSLQPRALVVEGCISKRVSHGQIKQIIRILSKALESCLKGPDTYNSQVLIEATVIALTKLQPLLNKDSPLHKALFWVAV
AVLQLDEVNLYSAGTALLEQNLHILDSLRIFNDKSPEEVFMAIRNPLEWHCKQMDHFVGLNENSNFNFALVGHLLKGYRH
PSPAIVARTVRILHILLTLVNKHRNCDKFEVNIQSVAYLAALLIVSEEVRSRCSLKHRKSLLLTDISMENVPMDTYPIHH
GDPSYRILKETQPWSSPKGSEGYLAATYPTVGQTSPRARKSMSLDMGQPSQANTKKLLGTRKSEDHLISDTKAPKRQEME
SGITIPPKMRRVAETDYEMETQRISSSQQHPHLRKVSVSESNVLLDEEVLTDPKIQALLLTVLATLVKYTTDEFDQRILY
EYLAEASVVFPKVFPVVHNLLDSKINTLLSLCQDPNLLNPIHGIVQSVVYHEESPPQYQTSYLQSFGENGLWRFAGPFSK
QTQIPDYAELIVKFLDALIDTYLPGIDEETSEESLLIPTSPYPPALQSQLSITANLNLSNSMISLATSQHSPGIDKENVE
LSPTIGHCNSGRTRHGSASQVQKQRSAGSFKRNSIKKIV
PIK3CA (UNIPROT accession no. P42336)
(SEQ ID NO: 36)
MPPRPSSGELWGIHLMPPRILVECLLPNGMIVTLECLREATLITIKHELFKEARKYPLHQLLQDESSYIFVSVIQEAERE
EFFDETRRLCDLRLFQPFLKVIEPVGNREEKILNREIGFAIGMPVCEFDMVKDPEVQDFRRNILNVCKEAVDLRDLNSPH
SRAMYVYPPNVESSPELPKHIYNKLDKGQIIVVIWVIVSPNNDKQKYILKINHDCVPEQVIAEAIRKKIRSMLLSSEQLK
LCVLEYQGKYILKVCGCDEYFLEKYPLSQYKYIRSCIMLGRMPNLMLMAKESLYSQLPMDCFIMPSYSRRISTATPYMNG
ETSTKSLWVINSALRIKILCATYVNVNIRDIDKIYVRTGIYHGGEPLCDNVNIQRVPCSNPRWNEWLNYDIYIPDLPRAA
RLCLSICSVKGRKGAKEEHCPLAWGNINLFDYTDILVSGKMALNLWPVPHGLEDLLNPIGVIGSNPNKETPCLELEFDWF
SSVVKFPDMSVIEEHANWSVSREAGESYSHAGLSNRLARDNELRENDKEQLKAISTRDPLSEITEQEKDFLWSHRHYCVT
IPEILPKLLLSVKWNSRDEVAQMYCLVKDWPPIKPEQAMELLDCNYPDPMVRGFAVRCLEKYLTDDKLSQYLIQLVQVLK
YEQYLDNLLVRELLKKALTNQRIGHFFEWHLKSEMHNKTVSQRFGLLLESYCRACGMYLKHLNRQVEAMEKLINLIDILK
QEKKDETQKVQMKFLVEQMRRPDFMDALQGFLSPLNPAHQLGNLRLEECRIMSSAKRPLWLNWENPDIMSELLFQNNEII
FKNGDDLRQDMLTLQIIRIMENIWQNQGLDLRMLPYGCLSIGDCVGLIEVVRNSHTIMQIQCKGGLKGALQFNSHILHQW
LKDKNKGEIYDAAIDLFIRSCAGYCVATFILGIGDRHNSNIMVKDDGQLFHIDFGHFLDHKKKKFGYKRERVPFVLIQDF
LIVISKGAQECTKIREFERFQEMCYKAYLAIRQHANLFINLFSMMLGSGMPELQSFDDIAYIRKTLALDKTEQEALEYFM
KQMNDAHHGGWTTKMDWIFHTIKQHALN
PTEN (UNIPROT accession no. P60484)
(SEQ ID NO: 37)
MTAIIKEIVSRNKRRYQEDGFDLDLTYIYPNIIAMGFPAERLEGVYRNNIDDVVRFLDSKHKNHYKIYNLCAERHYDTAK
FNCRVAQYPFEDHNPPQLELIKPFCEDLDQWLSEDDNHVAAIHCKAGKGRIGVMICAYLLHRGKFLKAQEALDFYGEVRT
RDKKGVTIPSQRRYVYYYSYLLKNHLDYRPVALLFHKMMFETIPMFSGGICNPQFVVCQLKVKIYSSNSGPIRREDKFMY
FEFPQPLPVCGDIKVEFFHKQNKMLKKDKMFHFWVNIFFIPGPEETSEKVENGSLCDQEIDSICSIERADNDKEYLVLIL
TKNDLDKANKDKANRYFSPNFKVKLYFIKTVEEPSNPEASSSTSVTPDVSDNEPDHYRYSDITDSDPENEPFDEDQHTQI
TKV
KRAS (UNIPROT accession no. P01116)
(SEQ ID NO: 38)
MTEYKLVVVGAGGVGKSALTIQLIQNHFVDEYDPTIEDSYRKQVVIDGETCLLDILDTAGQEEYSAMRDQYMRTGEGFLC
VFAINNIKSFEDIHHYREQIKRVKDSEDVPMVLVGNKCDLPSRTVDTKQAQDLARSYGIPFIETSAKTRQRVEDAFYILV
REIRQYRLKKISKEEKTPGCVKIKKCIIM
GATA3 (UNIPROT accession no. P23771)
(SEQ ID NO: 39)
MEVTADQPRWVSHHHPAVLNGQHPDTHHPGLSHSYMDAAQYPLPEEVDVLFNIDGQGNHVPPYYGNSVRATVQRYPPTHH
GSQVCRPPLLHGSLPWLDGGKALGSHHTASPWNLSPFSKTSIHHGSPGPLSVYPPASSSSLSGGHASPHLFTEPPIPPKD
VSPDPSLSTPGSAGSARQDEKECLKYQVPLPDSMKLESSHSRGSMTALGGASSSTHHPITTYPPYVPEYSSGLFPPSSLL
GGSPIGEGCKSRPKARSSIGRECVNCGATSTPLWRRDGIGHYLCNACGLYHKMNGQNRPLIKPKRRLSAARRAGTSCANC
QTTITTLWRRNANGDPVCNACGLYYKLHNINRPLIMKKEGIQTRNRKMSSKSKKCKKVHDSLEDFPKNSSFNPAALSRHM
SSLSHISPFSHSSHMLITPIPMHPPSSLSFGPHHPSSMVTAMG
MAP3K1 (UNIPROT accession no. Q13233)
(SEQ ID NO: 40)
MAAAAGNRASSSGFPGARATSPEAGGGGGALKASSAPAAAAGLLREAGSGGRERADWRRRQLRKVRSVELDQLPEQPLFL
AASPPASSTSPSPEPADAAGSGTGFQPVAVPPPHGAASRGGAHLTESVAAPDSGASSPAAAEPGEKRAPAAEPSPAAAPA
GREMENKETLKGLHKMDDRPEERMIREKLKATCMPAWKHEWLERRNRRGPVVVKPIPVKGDGSEMNHLAAESPGEVQASA
ASPASKGRRSPSPGNSPSGRTVKSESPGVRRKRVSPVPFQSGRITPPRRAPSPDGFSPYSPEETNRRVNKVMRARLYLLQ
QIGPNSFLIGGDSPDNKYRVFIGPQNCSCARGTECIHLLFVMLRVFQLEPSDPMLWRKTLKNFEVESLFQKYHSRRSSRI
KAPSRNTIQKFVSRMSNSHTLSSSSTSTSSSENSIKDEEEQMCPICLLGMLDEESLTVCEDGCRNKLHHHCMSIWAEECR
RNREPLICPLCRSKWRSHDFYSHELSSPVDSPSSLRAAQQQTVQQQPLAGSRRNQESNFNLTHYGTQQIPPAYKDLAEPW
IQVFGMELVGCLFSRNWNVREMALRRLSHDVSGALLLANGESTGNSGGSSGSSPSGGATSGSSQTSISGDVVEACCSVLS
MVCADPVYKVYVAALKTLRAMLVYTPCHSLAERIKLQRLLQPVVDTILVKCADANSRTSQLSISTLLELCKGQAGELAVG
REILKAGSIGIGGVDYVLNCILGNQTESNNWQELLGRLCLIDRLLLEFPAEFYPHIVSTDVSQAEPVEIRYKKLLSLLTF
ALQSIDNSHSMVGKLSRRIYLSSARMVTTVPHVFSKLLEMLSVSSSTHFTRMRRRLMAIADEVEIAEATQLGVEDTLDGQ
QDSFLQASVPNNYLETTENSSPECTVHLEKTGKGLCATKLSASSEDISERLASISVGPSSSTTTTTTTTEQPKPMVQTKG
RPHSQCLNSSPLSHHSQLMFPALSTPSSSTPSVPAGTATDVSKHRLQGFIPCRIPSASPQTQRKFSLQFHRNCPENKDSD
KLSPVFTQSRPLPSSNIHRPKPSRPTPGNTSKQGDPSKNSMTLDLNSSSKCDDSFGCSSNSSNAVIPSDETVFTPVEEKC
RLDVNTELNSSIEDLLEASMPSSDTTVTEKSEVAVLSPEKAENDDTYKDDVNHNQKCKEKMEAEEEEALAIAMAMSASQD
ALPIVPQLQVENGEDIIIIQQDTPETLPGHTKAKQPYREDTEWLKGQQIGLGAFSSCYQAQDVGTGTLMAVKQVTYVRNT
SSEQEEVVEALREEIRMMSHLNHPNIIRMLGATCEKSNYNLFIEWMAGGSVAHLLSKYGAFKESVVINYTEQLLRGLSYL
HENQIIHRDVKGANLLIDSTGQRLRIADFGAAARLASKGTGAGEFQGQLLGTIAFMAPEVLRGQQYGRSCDVWSVGCAII
EMACAKPPWNAEKHSNHLALIFKIASATTAPSIPSHLSPGLRDVALRCLELQPQDRPPSRELLKHPVERTTW
APC (UNIPROT accession no. P25054)
(SEQ ID NO: 41)
MAAASYDQLLKQVEALKMENSNLRQELEDNSNHLTKLETEASNMKEVLKQLQGSIEDEAMASSGQIDLLERLKELNLDSS
NFPGVKLRSKMSLRSYGSREGSVSSRSGECSPVPMGSFPRRGEVNGSRESTGYLEELEKERSLLLADLDKEEKEKDWYYA
QLQNLTKRIDSLPLTENFSLQTDMTRRQLEYEARQIRVAMEEQLGTCQDMEKRAQRRIARIQQIEKDILRIRQLLQSQAT
EAERSSQNKHETGSHDAERQNEGQGVGEINMATSGNGQGSTTRMDHETASVLSSSSTHSAPRRLTSHLGTKVEMVYSLLS
MLGTHDKDDMSRTLLAMSSSQDSCISMRQSGCLPLLIQLLHGNDKDSVLLGNSRGSKEARARASAALHNIIHSQPDDKRG
RREIRVLHLLEQTRAYCETCWEWQEAHEPGMDQDKNPMPAPVEHQICPAVCVLMKLSFDEEHRHAMNELGGLQATAELLQ
VDCEMYGLTNDHYSITLRRYAGMALTNLTEGDVANKATLCSMKGCMRALVAQLKSESEDLQQVIASVLRNLSWRADVNSK
KTLREVGSVKALMECALEVKKESTLKSVLSALWNLSAHCTENKADICAVDGALAFLVGTLTYRSQTNTLAIIESGGGILR
NVSSLIATNEDHRQILRENNCLQTLLQHLKSHSLTIVSNACGTLWNLSARNPKDQEALWDMGAVSMLKNLIHSKHKMIAM
GSAAALRNLMANRPAKYKDANIMSPGSSLPSLHVRKQKALEAELDAQHLSETFDNIDNLSPKASHRSKQRHKQSLYGDYV
FDTNRHDDNRSDNENTGNMTVLSPYLNTTVLPSSSSSRGSLDSSRSEKDRSLERERGIGLGNYHPATENPGTSSKRGLQI
STTAAQIAKVMEEVSAIHTSQEDRSSGSTTELHCVTDERNALRRSSAAHTHSNTYNFTKSENSNRTCSMPYAKLEYKRSS
NDSLNSVSSSDGYGKRGQMKPSIESYSEDDESKFCSYGQYPADLAHKIHSANHMDDNDGELDTPINYSLKYSDEQLNSGR
QSPSQNERWARPKHIIEDEIKQSEQRQSRNQSTTYPVYTESTDDKHLKFQPHFGQQECVSPYRSRGANGSETNRVGSNHG
INQNVSQSLCQEDDYEDDKPTNYSERYSEEEQHEEEERPTNYSIKYNEEKRHVDQPIDYSLKYATDIPSSQKQSFSFSKS
SSGQSSKTEHMSSSSENTSTPSSNAKRQNQLHPSSAQSRSGQPQKAATCKVSSINQETIQTYCVEDTPICFSRCSSLSSL
SSAEDEIGCNQTTQEADSANTLQIAEIKEKIGTRSAEDPVSEVPAVSQHPRTKSSRLQGSSLSSESARHKAVEFSSGAKS
PSKSGAQTPKSPPEHYVQETPLMFSRCTSVSSLDSFESRSIASSVQSEPCSGMVSGIISPSDLPDSPGQTMPPSRSKTPP
PPPQTAQTKREVPKNKAPTAEKRESGPKQAAVNAAVQRVQVLPDADTLLHFATESTPDGESCSSSLSALSLDEPFIQKDV
ELRIMPPVQENDNGNETESEQPKESNENQEKEAEKTIDSEKDLLDDSDDDDIEILEECIISAMPTKSSRKAKKPAQTASK
LPPPVARKPSQLPVYKLLPSQNRLQPQKHVSFTPGDDMPRVYCVEGTPINFSTATSLSDLTIESPPNELAAGEGVRGGAQ
SGEFEKRDTIPTEGRSTDEAQGGKTSSVTIPELDDNKAEEGDILAECINSAMPKGKSHKPFRVKKIMDQVQQASASSSAP
NKNQLDGKKKKPTSPVKPIPQNTEYRTRVRKNADSKNNLNAERVFSDNKDSKKQNLKNNSKVFNDKLPNNEDRVRGSFAF
DSPHHYTPIEGTPYCFSRNDSLSSLDFDDDDVDLSREKAELRKAKENKESEAKVTSHTELTSNQQSANKTQATAKQPINR
GQPKPILQKQSTFPQSSKDIPDRGAATDEKLQNFAIENTPVCFSHNSSLSSLSDIDQENNNKENEPIKETEPPDSQGEPS
KPQASGYAPKSFHVEDTPVCFSRNSSLSSLSIDSEDDLLQECISSAMPKKKKPSRLKGDNEKHSPRNMGGILGEDLTLDL
KDIQRPDSEHGLSPDSENFDWKAIQEGANSIVSSLHQAAAAACLSRQASSDSDSILSLKSGISLGSPFHLTPDQEEKPFT
SNKGPRILKPGEKSTLETKKIESESKGIKGGKKVYKSLITGKVRSNSEISGQMKQPLQANMPSISRGRTMIHIPGVRNSS
SSTSPVSKKGPPLKTPASKSPSEGQTATTSPRGAKPSVKSELSPVARQTSQIGGSSKAPSRSGSRDSTPSRPAQQPLSRP
IQSPGRNSISPGRNGISPPNKLSQLPRTSSPSTASTKSSGSGKMSYTSPGRQMSQQNLTKQTGLSKNASSIPRSESASKG
LNQMNNGNGANKKVELSRMSSTKSSGSESDRSERPVLVRQSTFIKEAPSPTLRRKLEESASFESLSPSSRPASPTRSQAQ
TPVLSPSLPDMSLSTHSSVQAGGWRKLPPNLSPTIEYNDGRPAKRHDIARSHSESPSRLPINRSGTWKREHSKHSSSLPR
VSTWRRTGSSSSILSASSESSEKAKSEDEKHVNSISGTKQSKENQVSAKGTWRKIKENEFSPTNSTSQTVSSGATNGAES
KTLIYQMAPAVSKTEDVWVRIEDCPINNPRSGRSPTGNTPPVIDSVSEKANPNIKDSKDNQAKQNVGNGSVPMRTVGLEN
RLNSFIQVDAPDQKGTEIKPGQNNPVPVSETNESSIVERTPFSSSSSSKHSSPSGTVAARVTPFNYNPSPRKSSADSTSA
RPSQIPTPVNNNTKKRDSKTDSTESSGTQSPKRHSGSYLVTSV
FBXW7 (UNIPROT accession no. Q969H0)
(SEQ ID NO: 42)
MNQELLSVGSKRRRTGGSLRGNPSSSQVDEEQMNRVVEEEQQQQLRQQEEEHTARNGEVVGVEPRPGGQNDSQQGQLEEN
NNRFISVDEDSSGNQEEQEEDEEHAGEQDEEDEEEEEMDQESDDFDQSDDSSREDEHTHTNSVTNSSSIVDLPVHQLSSP
FYTKTTKMKRKLDHGSEVRSFSLGKKPCKVSEYTSTTGLVPCSATPTTFGDLRAANGQGQQRRRITSVQPPTGLQEWLKM
FQSWSGPEKLLALDELIDSCEPTQVKHMMQVIEPQFQRDFISLLPKELALYVLSFLEPKDLLQAAQTCRYWRILAEDNLL
WREKCKEEGIDEPLHIKRRKVIKPGFIHSPWKSAYIRQHRIDTNWRRGELKSPKVLKGHDDHVITCLQFCGNRIVSGSDD
NTLKVWSAVTGKCLRTLVGHTGGVWSSQMRDNIIISGSTDRTLKVWNAETGECIHTLYGHTSTVRCMHLHEKRVVSGSRD
ATLRVWDIETGQCLHVLMGHVAAVRCVQYDGRRVVSGAYDFMVKVWDPETETCLHTLQGHTNRVYSLQFDGIHVVSGSLD
TSIRVWDVETGNCIHTLTGHQSLTSGMELKDNILVSGNADSTVKIWDIKTGQCLQTLQGPNKHQSAVTCLQFNKNEVITS
SDDGTVKLWDLKTGEFIRNLVTLESGGSGGVVWRIRASNTKLVCAVGSRNGTEETKLLVLDFDVDMK
DNMT3A (UNIPROT accession no. Q9Y6K1)
(SEQ ID NO: 43)
MPAMPSSGPGDTSSSAAEREEDRKDGEEQEEPRGKEERQEPSTTARKVGRPGRKRKHPPVESGDTPKDPAVISKSPSMAQ
DSGASELLPNGDLEKRSEPQPEEGSPAGGQKGGAPAEGEGAAETLPEASRAVENGCCTPKEGRGAPAEAGKEQKETNIES
MKMEGSRGRLRGGLGWESSLRQRPMPRLTFQAGDPYYISKRKRDEWLARWKREAEKKAKVIAGMNAVEENQGPGESQKVE
EASPPAVQQPTDPASPTVATTPEPVGSDAGDKNATKAGDDEPEYEDGRGEGIGELVWGKLRGESWWPGRIVSWWMTGRSR
AAEGTRWVMWEGDGKESVVCVEKLMPLSSFCSAFHQATYNKQPMYRKAIYEVLQVASSRAGKLFPVCHDSDESDTAKAVE
VQNKPMIEWALGGFQPSGPKGLEPPEEEKNPYKEVYTDMWVEPEAAAYAPPPPAKKPRKSTAEKPKVKEIIDERTRERLV
YEVRQKCRNIEDICISCGSLNVTLEHPLFVGGMCQNCKNCFLECAYQYDDDGYQSYCTICCGGREVLMCGNNNCCRCFCV
ECVDLLVGPGAAQAAIKEDPWNCYMCGHKGTYGLLRRREDWPSRLQMFFANNHDQEFDPPKVYPPVPAEKRKPIRVLSLF
DGIATGLLVLKDLGIQVDRYIASEVCEDSITVGMVRHQGKIMYVGDVRSVTQKHIQEWGPFDLVIGGSPCNDLSIVNPAR
KGLYEGTGRLFFEFYRLLHDARPKEGDDRPFEWLFENVVAMGVSDKRDISRFLESNPVMIDAKEVSAAHRARYFWGNLPG
MNRPLASTVNDKLELQECLEHGRIAKFSKVRTITTRSNSIKQGKDQHFPVFMNEKEDILWCTEMERVEGFPVHYTDVSNM
SRLARQRLLGRSWSVPVIRHLFAPLKEYFACV
CREBBP (UNIPROT accession no. Q92793)
(SEQ ID NO: 44)
MAENLLDGPPNPKRAKLSSPGFSANDSTDFGSLFDLENDLPDELIPNGGELGLLNSGNLVPDAASKHKQLSELLRGGSGS
SINPGIGNVSASSPVQQGLGGQAQGQPNSANMASLSAMGKSPLSQGDSSAPSLPKQAASTSGPTPAASQALNPQAQKQVG
LATSSPATSQTGPGICMNANFNQTHPGLLNSNSGHSLINQASQGQAQVMNGSLGAAGRGRGAGMPYPTPAMQGASSSVLA
ETLTQVSPQMTGHAGLNTAQAGGMAKMGITGNTSPFGQPFSQAGGQPMGATGVNPQLASKQSMVNSLPTEPTDIKNTSVT
NVPNMSQMQTSVGIVPTQATATGPTADPEKRKLIQQQLVLLLHAHKCQRREQANGEVRACSLPHCRTMKNVLNHMTHCQA
GKACQVAHCASSRQIISHWKNCTRHDCPVCLPLKNASDKRNQQTILGSPASGIQNTIGSVGTGQQNATSLSNPNPIDPSS
MQRAYAALGLPYMNQPQTQLQPQVPGQQPAQPQTHQQMRTLNPLGNNPMNIPAGGITTDQQPPNLISESALPTSLGATNP
LMNDGSNSGNIGTLSTIPTAAPPSSTGVRKGWHEHVTQDLRSHLVHKLVQATEPTPDPAALKDRRMENLVAYAKKVEGDM
YESANSRDEYYHLLAEKIYKIQKELEEKRRSRLHKQGILGNQPALPAPGAQPPVIPQAQPVRPPNGPLSLPVNRMQVSQG
MNSFNPMSLGNVQLPQAPMGPRAASPMNHSVQMNSMGSVPGMATSPSRMPQPPNMMGAHTNNMMAQAPAQSQFLPQNQFP
SSSGAMSVGMGQPPAQTGVSQGQVPGAALPNPLNMLGPQASQLPCPPVTQSPLHPTPPPASTAAGMPSLQHTTPPGMTPP
QPAAPTQPSTPVSSSGQTPTPTPGSVPSATQTQSTPTVQAAAQAQVTPQPQTPVQPPSVATPQSSQQQPTPVHAQPPGTP
LSQAAASIDNRVPTPSSVASAETNSQQPGPDVPVLEMKTETQAEDTEPDPGESKGEPRSEMMEEDLQGASQVKEETDIAE
QKSEPMEVDEKKPEVKVEVKEEEESSSNGTASQSTSPSQPRKKIFKPEELRQALMPTLEALYRQDPESLPFRQPVDPQLL
GIPDYFDIVKNPMDLSTIKRKLDTGQYQEPWQYVDDVWLMENNAWLYNRKTSRVYKFCSKLAEVFEQEIDPVMQSLGYCC
GRKYEFSPQTLCCYGKQLCTIPRDAAYYSYQNRYHFCEKCFTEIQGENVTLGDDPSQPQTTISKDQFEKKKNDTLDPEPF
VDCKECGRKMHQICVLHYDIIWPSGFVCDNCLKKTGRPRKENKFSAKRLQTTRLGNHLEDRVNKFLRRQNHPEAGEVFVR
VVASSDKTVEVKPGMKSRFVDSGEMSESFPYRTKALFAFEEIDGVDVCFFGMHVQEYGSDCPPPNTRRVYISYLDSIHFF
RPRCLRTAVYHEILIGYLEYVKKLGYVTGHIWACPPSEGDDYIFHCHPPDQKIPKPKRLQEWYKKMLDKAFAERIIHDYK
DIFKQATEDRLTSAKELPYFEGDFWPNVLEESIKELEQEEEERKKEESTAASETTEGSQGDSKNAKKKNNKKTNKNKSSI
SRANKKKPSMPNVSNDLSQKLYATMEKHKEVFFVIHLHAGPVINTLPPIVDPDPLLSCDLMDGRDAFLTLARDKHWEFSS
LRRSKWSTLCMLVELHTQGQDRFVYTCNECKHHVETRWHCTVCEDYDLCINCYNTKSHAHKMVKWGLGLDDEGSSQGEPQ
SKSPQESRRLSIQRCIQSLVHACQCRNANCSLPSCQKMKRVVQHTKGCKRKTNGGCPVCKQLIALCCYHAKHCQENKCPV
PFCLNIKHKLRQQQIQHRLQQAQLMRRRMATMNTRNVPQQSLPSPTSAPPGTPTQQPSTPQTPQPPAQPQPSPVSMSPAG
FPSVARTQPPTTVSTGKPTSQVPAPPPPAQPPPAAVEAARQIEREAQQQQHLYRVNINNSMPPGRTGMGTPGSQMAPVSL
NVPRPNQVSGPVMPSMPPGQWQQAPLPQQQPMPGLPRPVISMQAQAAVAGPRMPSVQPPRSISPSALQDLLRTLKSPSSP
QQQQQVLNILKSNPQLMAAFIKQRTAKYVANQPGMQPQPGLQSQPGMQPQPGMHQQPSLQNLNAMQAGVPRPGVPPQQQA
MGGLNPQGQALNIMNPGHNPNMASMNPQYREMLRRQLLQQQQQQQQQQQQQQQQQQGSAGMAGGMAGHGQFQQPQGPGGY
PPAMQQQQRMQQHLPLQGSSMGQMAAQMGQLGQMGQPGLGADSTPNIQQALQQRILQQQQMKQQIGSPGQPNPMSPQQHM
LSGQPQASHLPGQQIATSLSNQVRSPAPVQSPRPQSQPPHSSPSPRIQPQPSPHHVSPQTGSPHPGLAVIMASSIDQGHL
GNPEQSAMLPQLNTPSRSALSSELSLVGDTTGDTLEKFVEGL
SF3B1 (UNIPROT accession no. O75533)
(SEQ ID NO: 45)
MAKIAKTHEDIEAQIREIQGKKAALDEAQGVGLDSTGYYDQEIYGGSDSRFAGYVISIAATELEDDDDDYSSSTSLLGQK
KPGYHAPVALLNDIPQSTEQYDPFAEHRPPKIADREDEYKKHRRIMIISPERLDPFADGGKTPDPKMNARTYMDVMREQH
LIKEEREIRQQLAEKAKAGELKVVNGAAASQPPSKRKRRWDQTADQTPGATPKKLSSWDQAETPGHTPSLRWDETPGRAK
GSETPGATPGSKIWDPIPSHIPAGAATPGRGDTPGHATPGHGGATSSARKNRWDETPKTERDTPGHGSGWAETPRIDRGG
DSIGETPTPGASKRKSRWDETPASQMGGSTPVLIPGKTPIGTPAMNMATPTPGHIMSMTPEQLQAWRWEREIDERNRPLS
DEELDAMFPEGYKVLPPPAGYVPIRTPARKLTATPTPLGGMTGFHMQTEDRIMKSVNDQPSGNLPFLKPDDIQYFDKLLV
DVDESTLSPEEQKERKIMKLLLKIKNGIPPMRKAALRQIIDKAREFGAGPLENQILPLLMSPTLEDQERHLLVKVIDRIL
YKLDDLVRPYVHKILVVIEPLLIDEDYYARVEGREIISNLAKAAGLATMISTMRPDIDNMDEYVRNITARAFAVVASALG
IPSLLPFLKAVCKSKKSWQARHIGIKIVQQIAILMGCAILPHLRSLVEIIEHGLVDEQQKVRTISALAIAALAEAATPYG
IESFDSVLKPLWKGIRQHRGKGLAAFLKAIGYLIPLMDAEYANYYTREVMLILIREFQSPDEEMKKIVLKVVKQCCGIDG
VEANYIKTEILPPFFKHFWQHRMALDRRNYRQLVDTIVELANKVGAAEIISRIVDDLKDEAEQYRKMVMETIEKIMGNLG
AADIDHKLEEQLIDGILYAFQEQTTEDSVMLNGFGTVVNALGKRVKPYLPQICGTVLWRLNNKSAKVRQQAADLISRTAV
VMKTCQEEKLMGHLGVVLYEYLGEEYPEVLGSILGALKAIVNVIGMHKMIPPIKDLLPRLIPILKNRHEKVQENCIDLVG
RIADRGAEYVSAREWMRICFELLELLKAHKKAIRRATVNIFGYIAKAIGPHDVLAILLNNLKVQERQNRVCTIVAIAIVA
ETCSPFTVLPALMNEYRVPELNVQNGVLKSLSFLFEYIGEMGKDYIYAVTPLLEDALMDRDLVHRQTASAVVQHMSLGVY
GFGCEDSLNHLLNYVWPNVFETSPHVIQAVMGALEGLRVAIGPCRMLQYCLQGLFHPARKVRDVYWKIYNSIYIGSQDAL
IAHYPRIYNDDKNTYIRYELDYIL

Identification of Tumor-Associated Splice Isoforms

The technology described herein can identify splice variants that are differentially expressed in tumor cells and non-tumor cells. Cancer specific isoforms derived from such differentially spliced mRNAs are identified in an unbiased manner by analyzing genome-scale isoform expression data. Median isoform expression values from large sets of tumor samples are compared to isoform expression data of normal samples. One tumor type is explored at a time, but the targets identified are often present in multiple tumor types. Tumor expression levels are compared to both tissue-matched normal expression values and a composite panel of all available normal samples. Isoforms that are not expressed in normal tissue but that are expressed in tumor tissue are identified as well as isoforms that are strongly expressed in tumor but expressed at minimal level in normal tissue. Target proteins are reported in a rank-order list sorted by highest tumor expression level and fold change compared to normal. Protein multi-sequence alignment is performed for all gene isoforms to identify unique protein peptides of the cancer specific isoform. Currently, the pipeline uses TCGA tumor and normal data as well as GTEX normal data.

Following the methods described above, cancer-specific isoforms were predicted and prioritized for Breast Cancer (BCA), ovarian cancer (OC), B-cell lymphomas, bladder urothelial carcinoma, and mesothelioma.
Breast Cancer (BCA)

Sodium/potassium-transporting ATPase subunit beta-1-interacting protein 1 (NKAIN1) splice variant uc001bsn (ENST00000263693) was analyzed. FIGS. 1A and 1B show differential expression results between normal tissues (GTEx database) versus cancer samples (TOGA database). The following is an alignment between the canonical form (top; SEQ ID NO:1) and the BCA-associated isoform (bottom; bold; SEQ ID NO:2 (unique sequence underlined)):

>uc010ogc
(SEQ ID NO: 1)
--------------------------------------------
MAVILGIFGTVQYRSRYLIL---------------------------
DRDFIMTFNTSLHRSWWMENGPGCLVTPVLNSRLALEDHHVISVTGCLLDYPYIEALSSALQIFLA
LFGFVFACYVSKVFLEEEDSFDFIGGFDSYGYQAPQKTSHLQLQPLYTSG
>uc001bsn
(SEQ ID NO: 2)
--------------------------------------------
MAVILGIFGTVQYRSRYLILYAAWLVLWVGWNAFIICFYLEVGQLSQDRDFIMTFNTSLHRSTAINNE
NGPGCLVTPVLNSRLALEDHHVISVTGCLLDYPYIEALSSALQIFLALFGFVFACYVSKVFLEEED
SFDFIGGFDSYGYQAPQKTSHLQLQPLYTSG

The tumor isoform has the following additional amino acid sequence: YAAWLVLVVVGWNAFIICFYLEVGQLSQ (SEQ ID NO:3). This sequence was used by the technology described herein to predict immunogenic peptides. Results from peptide prioritization analysis returned the following peptide: YLILYAAWLVLWVGWNAFIICFYLEV (SEQ ID NO:4), which contains peptides predicted to bind to the following HLA molecules:

| nM | | | SEQ |
|---|---|---|---|
| affinity | Allele | Sequence | ID NO |
| 9.16 | HLA-A*02:1220 | FIICFYLEV | 5 |
| 112.99 | HLA-A*24:026 | LYAAWLVLW | 6 |
| 270.71 | HLA-A*30:0217 | WNAFIICFY | 7 |
| 5.16 | HLA-A*68:0218 | NAFIICFYL | 8 |
| 146.88 | HLA-B*08:013 | YLILYAAWL | 9 |
| 116.76 | HLA-B*15:0112 | VLWVGWNAF | 10 |
| 16.58 | HLA-B*58:016 | LYAAWLVLW | 11 |
| 30.15 | HLA-DRB1*01:01 | AWLVLWVGWNAFIIC | 12 |
| 12.99 | HLA-DRB1*07:01 | LWVGWNAFIICFYLE | 13 |
| 129.13 | HLA-DRB1*08:02 | AAWLVLWVGWNAFII | 14 |
| 104.51 | HLA-DRB1*09:01 | VLWVGWNAFIICFYL | 15 |
| 14.48 | HLA-DRB1*15:01 | WVGWNAFIICFYLEV | 16 |

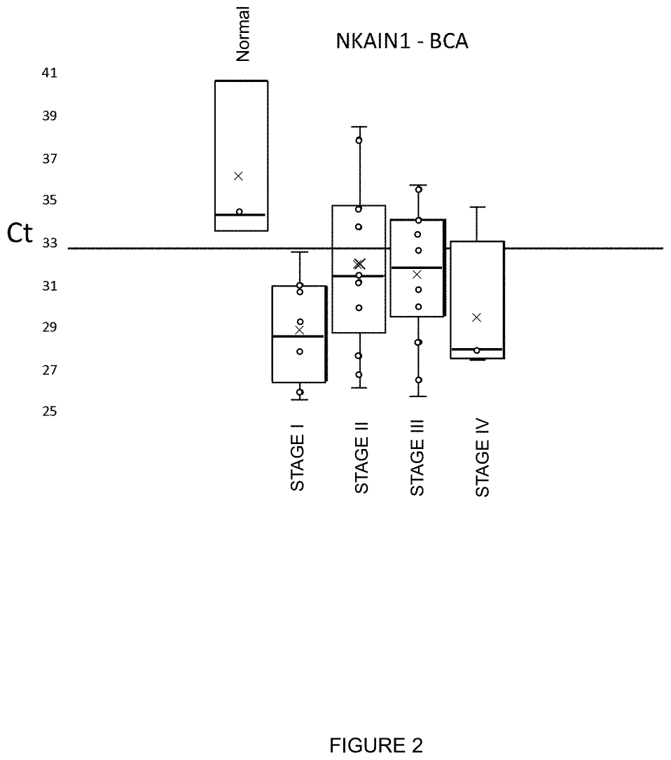

The differential expression of the uc001bsn splice variant was validated on a panel of 40 BCA samples and 8 normal breast samples by splice-specific qRT-PCR, where one of the two primers maps on the tumor-associated-specific sequence of the cDNA. FIG. 2 shows the results expressed as threshold cycle (Ct) using a SYBR-green-based qPCR assay.
Ovarian Cancer (OC)

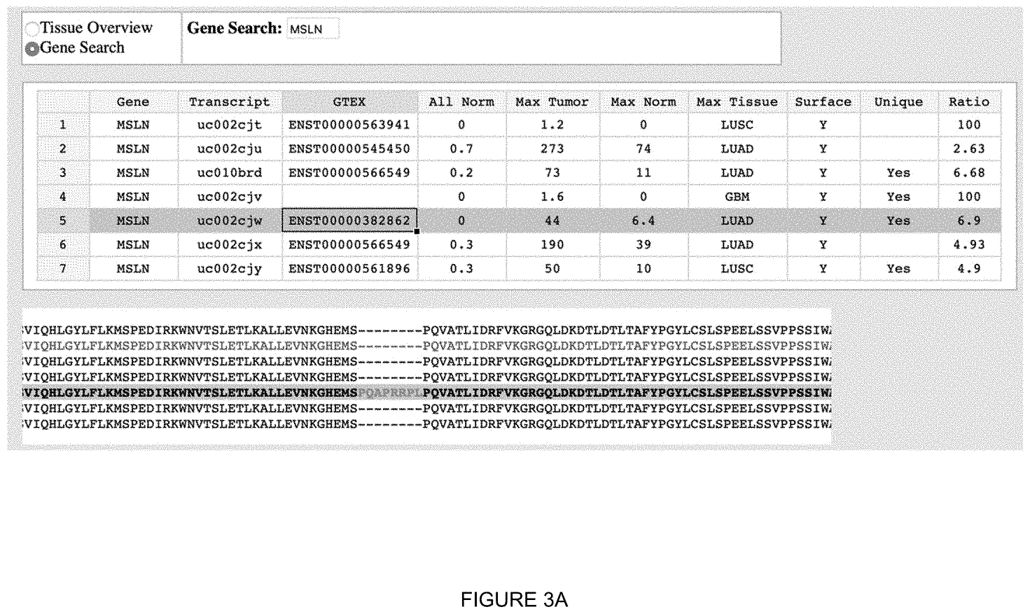

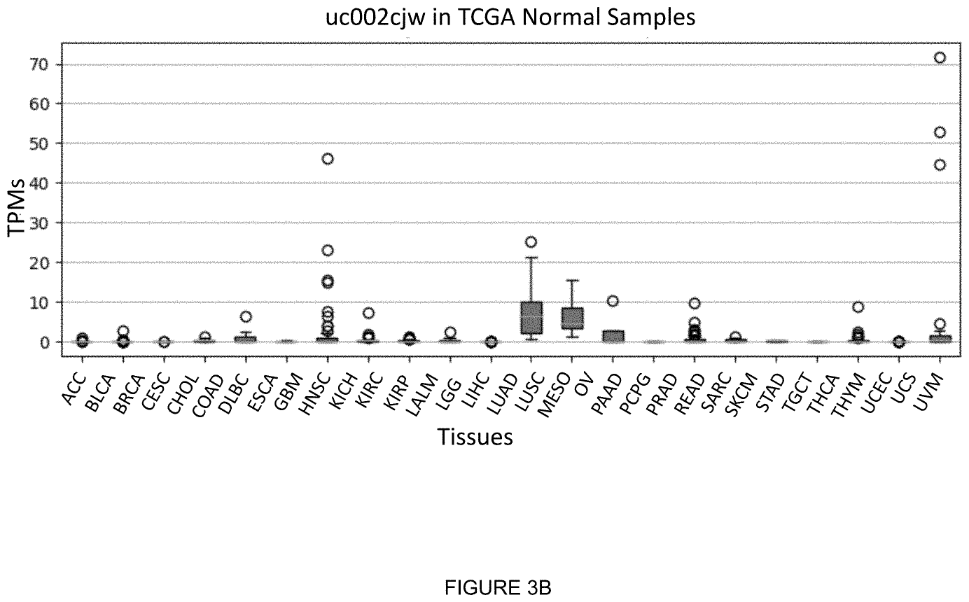

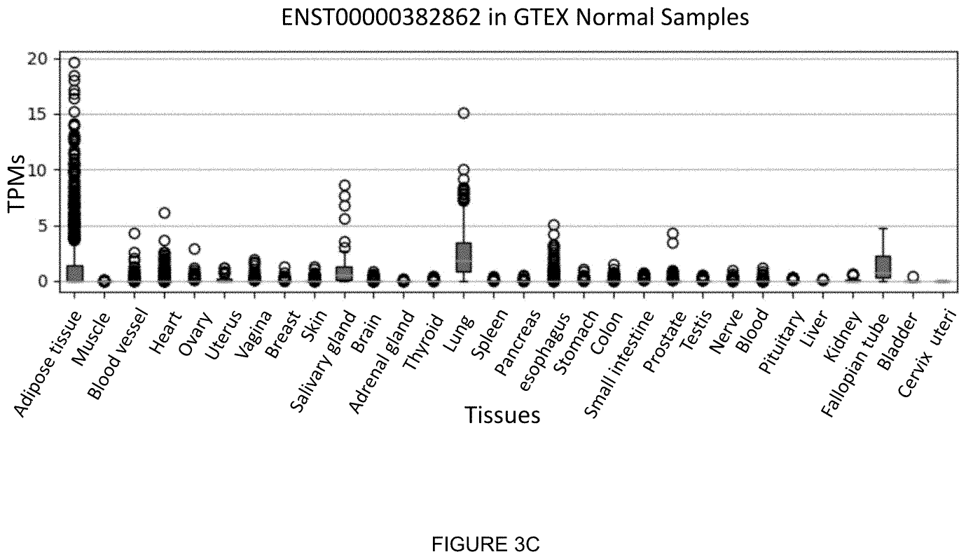

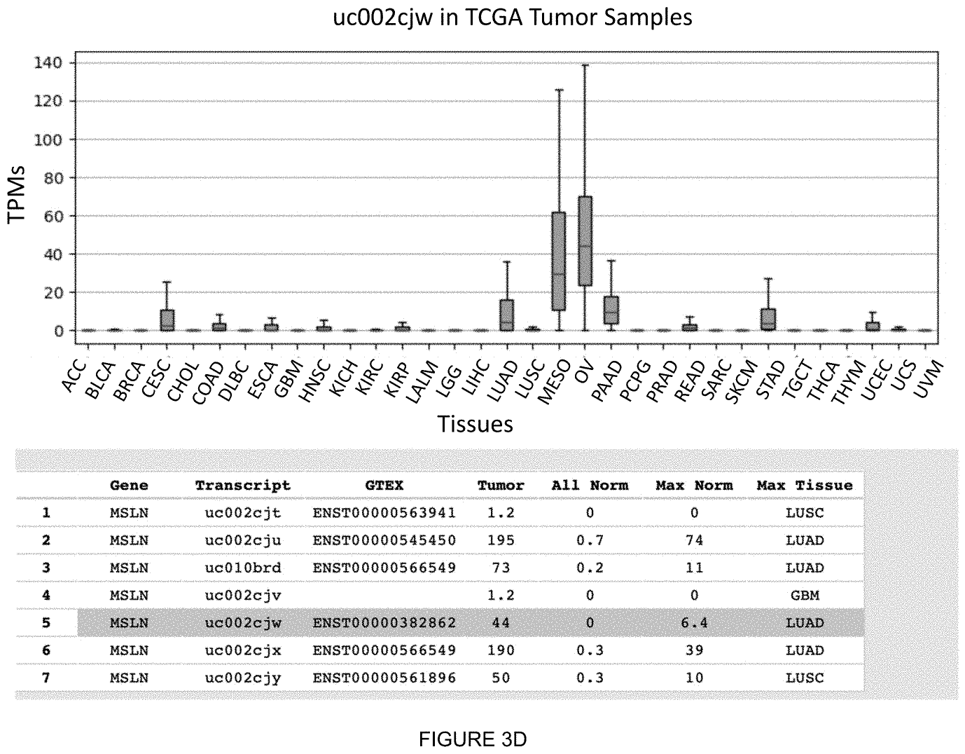

Mesothelin (MSLN) splice variant uc002cjw (ENST00000382862) was analyzed (graphical interface for the analysis (search gene option) and sequence alignments for isoform variants is shown in FIG. 3A). FIG. 3B to FIG. 3D show differential expression results between normal tissues (TOGA database, GTEx database) versus cancer samples (TOGA database).

The following is an alignment between the canonical form (top; SEQ ID NO:17); the BCA-associated isoform (second from top; bold; SEQ ID NO:18 (unique sequence underlined)); and five minor isoforms (third to seventh from top; SEQ ID NOs: 50-54, respectively)

```
>uc002cju
(SEQ ID NO: 17)
MALPTARPLLGSCGTPALGSLLFLLFSLGWVQPSRTLAGETGQEAAPLDGVLANPPNISSL
SPRQLLGFPCAEVSGLSTERVRELAVALAQKNVKLSTEQLRCLAHRLSEPPEDLDALPLDLLLFLN
PDAFSGPQACTRFFSRITKANVDLLPRGAPERQRLLPAALACWGVRGSLLSEADVRALGGLACDLP
GRFVAESAEVLLPRLVSCPGPLDQDQQEAARAALQGGGPPYGPPSTWSVSTMDALRGLLPVLGQPI
IRSIPQGIVAAWRQRSSRDPSWRQPERTILRPRFRREVEKTACPSGKKAREIDESLIFYKKWELEA
CVDAALLATQMDRVNAIPFTYEQLDVLKHKLDELYPQGYPESVIQHLGYLFLKMSPEDIRKWNVTS
LETLKALLEVNKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQEALSGTP-----
-----------------------------CLLGPGPVLTVLALLLASTLA----------------
----------------------
>uc002cjw
(SEQ ID NO: 18)
MALPTARPLLGSCGTPALGSLLFLLFSLGWVQPSRTLAGETGQEAAPLDGVLANPPNISS
LSPRQLLGFPCAEVSGLSTERVRELAVALAQKNVKLSTEQLRCLAHRLSEPPEDLDALPLDLLLFL
NPDAFSGPQACTRFFSRITKANVDLLPRGAPERQRLLPAALACWGVRGSLLSEADVRALGGLACDL
PGRFVAESAEVLLPRLVSCPGPLDQDQQEAARAALQGGGPPYGPPSTWSVSTMDALRGLLPVLGQP
IIRSIPQGIVAAWRQRSSRDPSWRQPERTILRPRFRREVEKTACPSGKKAREIDESLIFYKKWELE
ACVDAALLATQMDRVNAIPFTYEQLDVLKHKLDELYPQGYPESVIQHLGYLFLKMSPEDIRKWNVT
SLETLKALLEVNKGHEMSPQAPRRPLPQVATLIDRFVKGRGQLDKDTLDTLTAFYPGYLCSLSPEE
LSSVPPSSIWAVRPQDLDTCDPRQLDVLYPKARLAFQNMNGSEYFVKIQSFLGGAPTEDLKALSQQ
NVSMDLATFMKLRTDAVLPLTVAEVQKLLGPHVEGLKAEERHRPVRDWILRQRQDDLDTLGLGLQG
GIPNGYLVLDLSMQEALSGTP----------------------------------
CLLGPGPVLTVLALLLASTLA--------------------------------------
>uc002cjt
(SEQ ID NO: 50)
MALPTARPLLGSCGTPALGSLLFLLFSLGWVQPSRTLAGETGQEAAPLDGVLANPPNISS
LSPRQLLGFPCAEVSGLSTERVRELAVALAQKNVKLSTEQLRCLAHRLSEPPEDLDALPLDLLLFL
NPDAFSGPQACTRFFSRITKANVDLLPRGAPERQRLLPAALACWGVRGSLLSEADVRALGGLACDL
PGRFVAESAEVLLPRLVSCPGPLDQDQQEAARAALQGGGPPYGPPSTWSVSTMDALRGLLPVLGQP
IIRSIPQGIVAAWRQRSSRDPSWRQPERTILRPRFRREVEKTACPSGKKAREIDESLIFYKKWELE
ACVDAALLATQMDRVNAIPFTYEQLDVLKHKLDELYPQGYPESVIQHLGYLFLKMSPEDIRKWNVT
SLETLKALLEVNKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQEALSGTP-----
-----------------------------CLLGPGPVLTVLALLLASTLA----------------
>uc010brd
(SEQ ID NO: 51)
            MALPTARPLLGSCGTPALGSLLFLLFSLGWVQPSRTLAGETGQ-
AAPLDGVLANPPNISSLSPRQLLGFPCAEVSGLSTERVRELAVALAQKNVKLSTEQLRCLAHRLSE
PPEDLDALPLDLLLFLNPDAFSGPQACTRFFSRITKANVDLLPRGAPERQRLLPAALACWGVRGSL
LSEADVRALGGLACDLPGRFVAESAEVLLPRLVSCPGPLDQDQQEAARAALQGGGPPYGPPSTWSV
STMDALRGLLPVLGQPIIRSIPQGIVAAWRQRSSRDPSWRQPERTILRPRFRREVEKTACPSGKKA
REIDESLIFYKKWELEACVDAALLATQMDRVNAIPFTYEQLDVLKHKLDELYPQGYPESVIQHLGY
LFLKMSPEDIRKWNVTSLETLKALLEVNKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQEALSGTP-----
-----------------------------CLLGPGPVLTVLALLLASTLA----------------
>uc002cjv
(SEQ ID NO: 52)
MALPTARPLLGSCGTPALGSLLFLLFSLGWVQPSRTLAGETGQEAAPLDGVLANPPNISS
LSPRQLLGFPCAEVSGLSTERVRELAVALAQKNVKLSTEQLRCLAHRLSEPPEDLDALPLDLLLFL
NPDAFSGPQACTRFFSRITKANVDLLPRGAPERQRLLPAALACWGVRGSLLSEADVRALGGLACDL
PGRFVAESAEVLLPRLVSCPGPLDQDQQEAARAALQGGGPPYGPPSTWSVSTMDALRGLLPVLGQP
IIRSIPQGIVAAWRQRSSRDPSWRQPERTILRPRFRREVEKTACPSGKKAREIDESLIFYKKWELE
ACVDAALLATQMDRVNAIPFTYEQLDVLKHKLDELYPQGYPESVIQHLGYLFLKMSPEDIRKWNVT
SLETLKALLEVNKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQ------------
--------------------------------GPGPVLTVLALLLASTLA----------------
>uc002cjx
(SEQ ID NO: 53)
            MALPTARPLLGSCGTPALGSLLFLLFSLGWVQPSRTLAGETGQEAAPLDGVLANPPNISS
LSPRQLLGFPCAEVSGLSTERVRELAVALAQKNVKLSTEQLRCLAHRLSEPPEDLDALPLDLLLFL
NPDAFSGPQACTRFFSRITKANVDLLPRGAPERQRLLPAALACWGVRGSLLSEADVRALGGLACDL
PGRFVAESAEVLLPRLVSCPGPLDQDQQEAARAALQGGGPPYGPPSTWSVSTMDALRGLLPVLGQP
IIRSIPQGIVAAWRQRSSRDPSWRQPERTILRPRFRREVEKTACPSGKKAREIDESLIFYKKWELE
ACVDAALLATQMDRVNAIPFTYEQLDVLKHKLDELYPQGYPESVIQHLGYLFLKMSPEDIRKWNVT
SLETLKALLEVNKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQEALSGTP-----
-----------------------------CLLGPGPVLTVLALLLASTLA----------------
----------------------
>uc002cjy
(SEQ ID NO: 54)
            ------------------------------------------------------------
------------------------------------------------------------------
------------------------------------------------------------------
------------------------------------------------------------------
------------------------------------------------------------------
-----------
MDRVNAIPFTYEQLDVLKHKLDELYPQGYPESVIQHLGYLFLKMSPEDIRKWNVTSLETLKALLEV
NKGHEMS--------
PQVATLIDRFVKGRGQLDKDTLDTLTAFYPGYLCSLSPEELSSVPPSSIWAVRPQDLDTCDPRQLD
VLYPKARLAFQNMNGSEYFVKIQSFLGGAPTEDLKALSQQNVSMDLATFMKLRTDAVLPLTVAEVQ
KLLGPHVEGLKAEERHRPVRDWILRQRQDDLDTLGLGLQGGIPNGYLVLDLSMQGGRGGQARAGGR
AGGVEVGALSHPSLCRGPLGDALPPRTWTCSHRPGTAPSLHPGLRAPLPCWPQPCWGSPPGQEQAR
VVPVPPQENSRSVNGNMPPADT
```

The tumor isoform has the following additional amino acid sequence: PQAPRRPL (SEQ ID NO:19). This information was used by the technology described herein to predict immunogenic peptides. Results from peptide prioritization analysis returned the following peptide: VNKGHEMSPQAPRRPLPQVATLI (SEQ ID NO:20), which contains peptides predicted to bind to the following HLA molecules:

| nM | | | SEQ |
|---|---|---|---|
| affinity | Allele | Sequence | ID NO |
| 356.11 | HLA-A*24:0214 | PLPQVATLI | 21 |
| 351.88 | HLA-A*02:0214 | PLPQVATLI | 21 |
| 4.14 | HLA-B*07:027 | SPQAPRRPL | 22 |
| 163.84 | HLA-DRB1*04:01 | VNKGHEMSPQAPRRP | 23 |
| 146.74 | HLA-DRB1*04:04 | NKGHEMSPQAPRRPL | 24 |
| 359.27 | HLA-DRB1*09:01 | NKGHEMSPQAPRRPL | 24 |

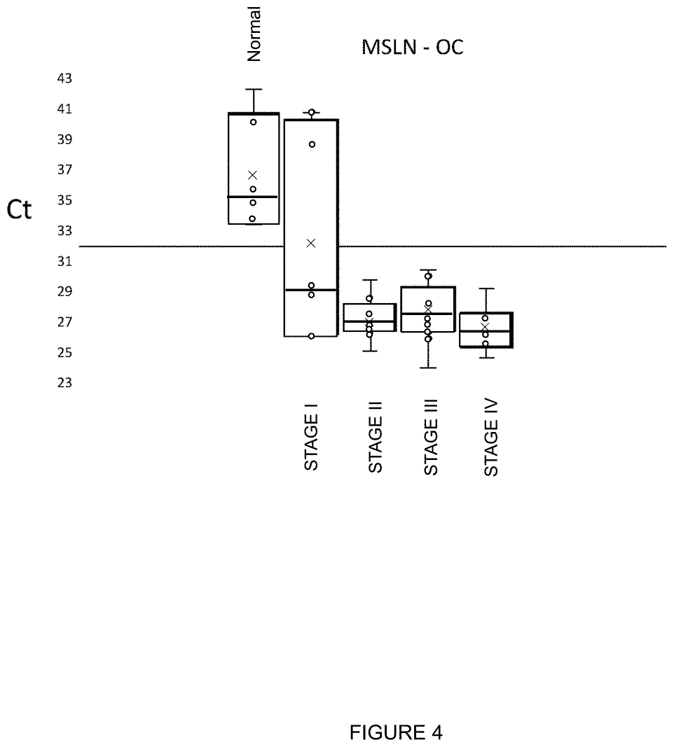

The differential expression of the uc002cjw splice variant was validated on a panel of 40 OC samples and 8 normal ovary samples by splice-specific qRT-PCR, where one of the two primers maps on the tumor-associated-specific sequence of the cDNA. FIG. 4 shows the results expressed as threshold cycle (Ct) using a SYBR-green-based qPCR assay.

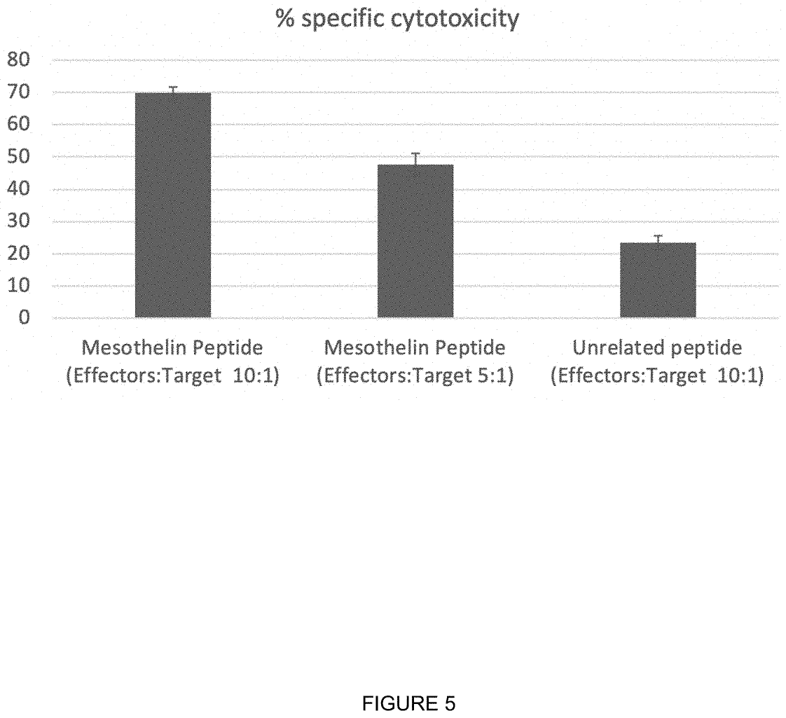

The peptide unique to the isoform variant (i.e., PQAPRRPL (SEQ ID NO: 19)) was then tested for immunogenicity using a dendritic cell (DC)- T-cell co-culture system as previously described (Chiriva-Internati et al. Blood. 2002 Aug. 1;100(3):961-5). A standard 4-hour Calcein-AM-release assay was performed to determine the cytotoxic activity of the IsoMSLN peptide stimulated T cells. Autologous DCs pulsed with the IsoMSLN peptide or with an unrelated peptide (derived from HIV), were used as target cells (at an effector-target cell [E/T] ratio of 10:1 and 5:1). FIG. 5 shows the percent specific killing, confirming the peptide predicted by the technology described herein was immunogenic when tested on the peripheral blood mononuclear cells of a healthy individual.

The transcriptomics data, based on RNA-seq data mining using the technology described herein, was confirmed at the protein level, by analyzing a mass spectrometry dataset containing ovarian cancer samples and adjacent non-tumoral tissues. Data from the clinical study “S038 Confirmatory Study” were then downloaded from CPTAC (World Wide Web Uniform Resource Locator cptac-data-portal.georgetown.edu/study-summary/5038). 13 datasets were analyzed, which included 94 ovarian tumor and 23 ovarian normal tissue samples from the same group of ovarian cancer patients. Data parsing and data quality control data were processed by MS Biowork through the MaxQuant software v1.6.2.3. As shown in the table below, the peptide that is present only in the isoform variant was detected in 71% of tumor tissues and in 61% of normal tissues, while a peptide corresponding to the canonical form of Mesothelin was detected in 100% of both tumors and normal samples, indicating that, as predicted by the technology described herein, the isoform expression was more selective for cancer tissues.

| | Peptide | MSLN | % of Adjacent | % of Tumor |
|---|---|---|---|---|
| Protein | (SEQ ID NO) | Transcripts | Normal Tissues | Tissues |
| IsoMSLN | RPLPQVATLIDR (25) | uc002cjw | 61 | 71 |
| MSLN | GHEMSPQVATLIDR (26) | uc002cjv; | 100 | 100 |
| | | uc010brd; | | |
| | | uc002cju; | | |
| | | uc002cjy | | |

Ovarian Cancer (OC), Bladder Urothelial Carcinoma, and Mesothelioma

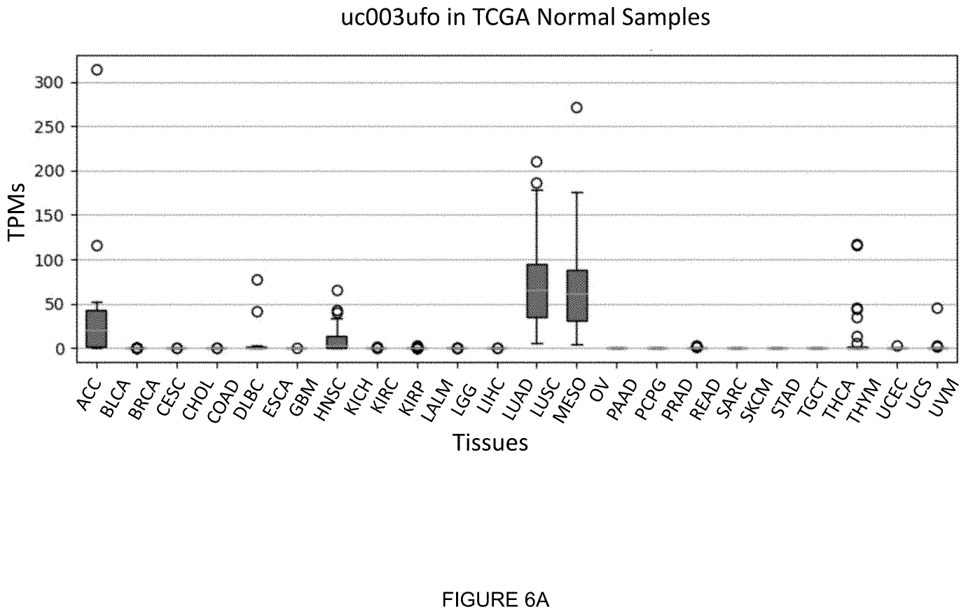

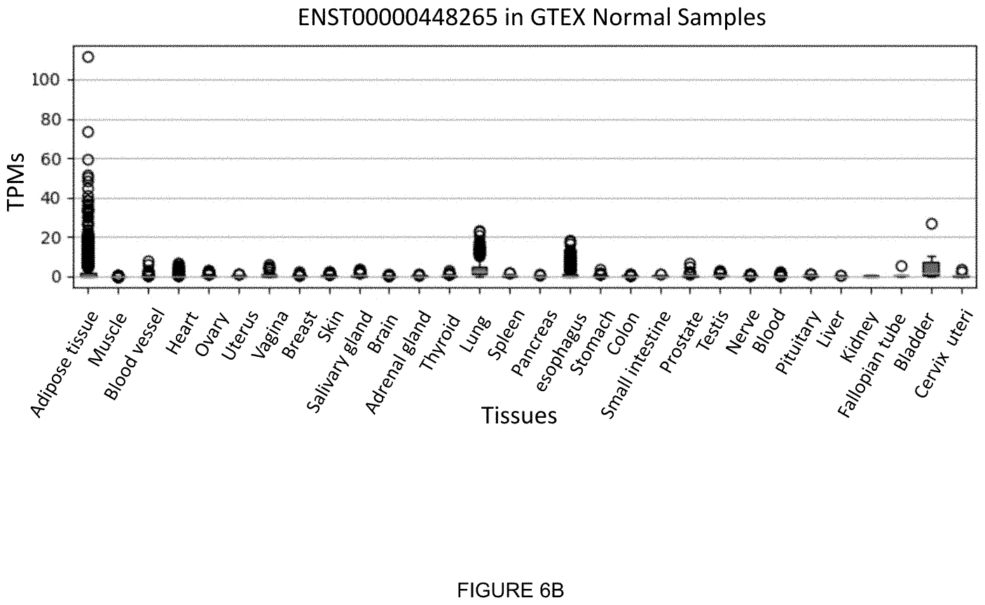

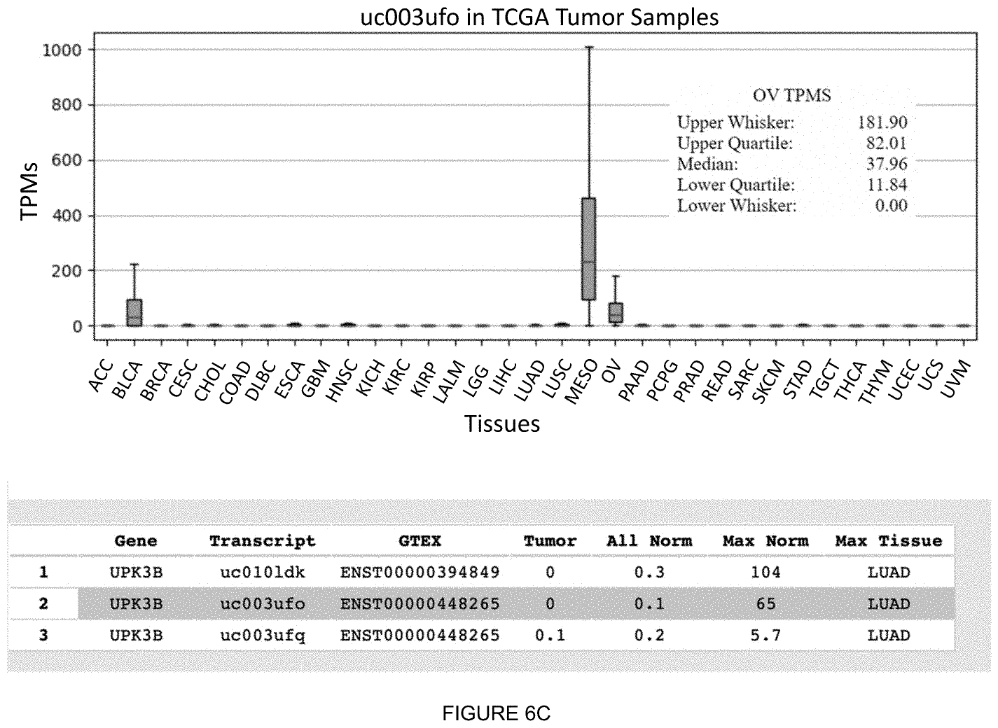

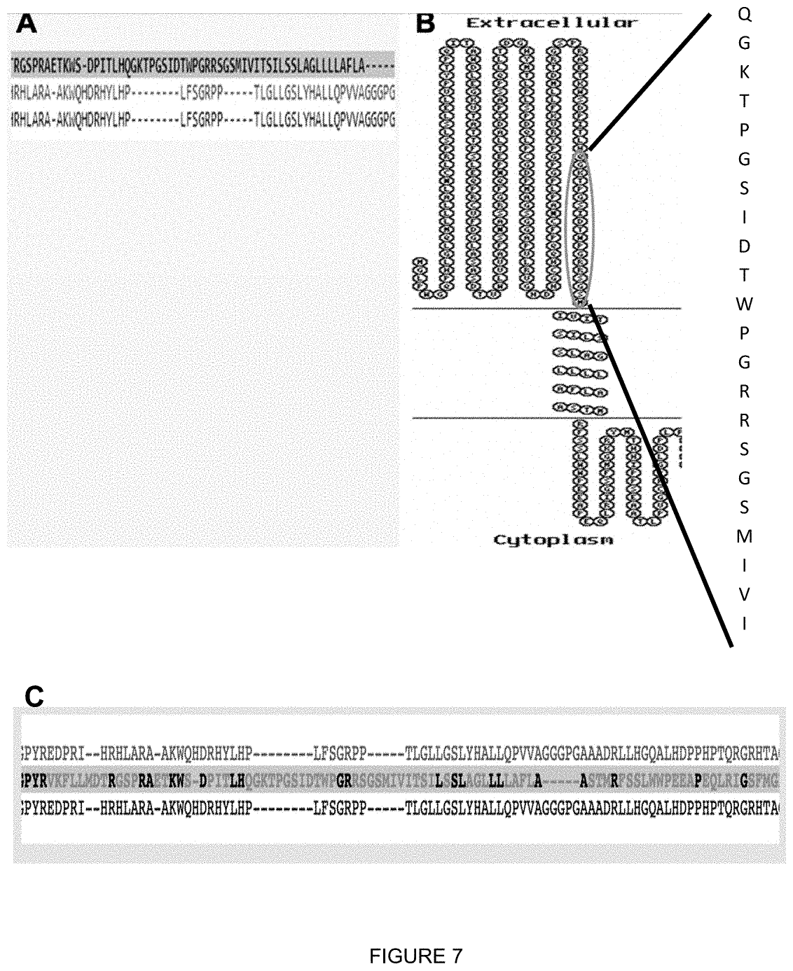

Uroplakin3b (UPK3B) splice variant uc003ufo (ENST00000448265) was analyzed. FIGS. 6A-6C shows differential expression results between normal tissues (TOGA database, GTEx database) versus tumor samples (TOGA database). As shown in FIG. 7, the UPK3B isoform identified by the technology described herein has a unique amino acid sequence that has a predicted location in the extracellular domain of the protein.

The following is an alignment between the canonical form (top; SEQ ID NO:46), tumor-associated isoform (middle; bold; SEQ ID NO:47 (unique sequence underlined)), and a minor isoform (bottom; SEQ ID NO:47):

>uc010ldk
(SEQ ID NO: 46)
MGLPWGQPHLGLQMLLLALNCLRPSLSL--------------------------
-----------------------------
ELVPYTPQITAWDLEGKVTATTFSLEQPRCVFDGLASASDTVWLVVAFSNASRGFQNPETLADIPA
SPQLLTDGHYMTLPLSPDQLPCGDPMAGSGGAPVLRVGHDHGCHQQPFCNAPLPGPGPYREDPRI-
-HRHLARA-AKWQHDRHYLHP--------LFSGRPP-----
TLGLLGSLYHALLQPVVAGGGPGAAADRLLHGQALHDPPHPTQRGRHTAGGLQAWPGPPPQPQPLA
WPLCMGLGEMGRWE-
>uc003ufo
(SEQ ID NO: 47)
MGLPWGQPHLGLQMLLLALNCLRPSLSL--------------------------
-----------------------------
ELVPYTPQITAWDLEGKVTATTFSLEQPRCVFDGLASASDTVWLVVAFSNASRGFQNPETLADIPA
SPQLLTDGHYMTLPLSPDQLPCGDPMAGSGGAPVLRVGHDHGCHQQPFCNAPLPGPGPYRVKFLLM
DTRGSPRAETKWS-DPITLHQGKTPGSIDTWPGRRSGSMIVITSILSSLAGLLLLAFLA-----
ASTMRFSSLWWPEEAPEQLRIGSFMGKRYMTHHIPPSEAATLPVGCKPGLDPLPSLSP
>uc003ufq
(SEQ ID NO: 48)
MGLPWGQPHLGLQMLLLALNCLRPSLSLGEWGSWMDASSQTQGAGGPAGVIGPWAPAPLR
LGEAAPGTPTPVSVAHLLSPVATELVPYTPQITAWDLEGKVTATTFSLEQPRCVFDGLASASDTVW
LVVAFSNASRGFQNPETLADIPASPQLLTDGHYMTLPLSPDQLPCGDPMAGSGGAPVLRVGHDHGC
HQQPFCNAPLPGPGPYREDPRI--HRHLARA-AKWQHDRHYLHP--------LFSGRPP-----
TLGLLGSLYHALLQPVVAGGGPGAAADRLLHGQALHDPPHPTQRGRHTAGGLQAWPGPPPQPQPLA
WPLCMGLGEMGRRE-

The tumor isoform has the following additional amino acid sequence:

(SEQ ID NO: 49)
VKFLLMDTRGSPRAETKWS-DPITLHQGKTPGSIDTWPGRRSGSMIVITSILSSLAGLLLLAFLA-----
ASTMRFSSLWWPEEAPEQLRIGSFMGKRYMTHHIPPSEAATLPVGCKPGLDPLPSLSP

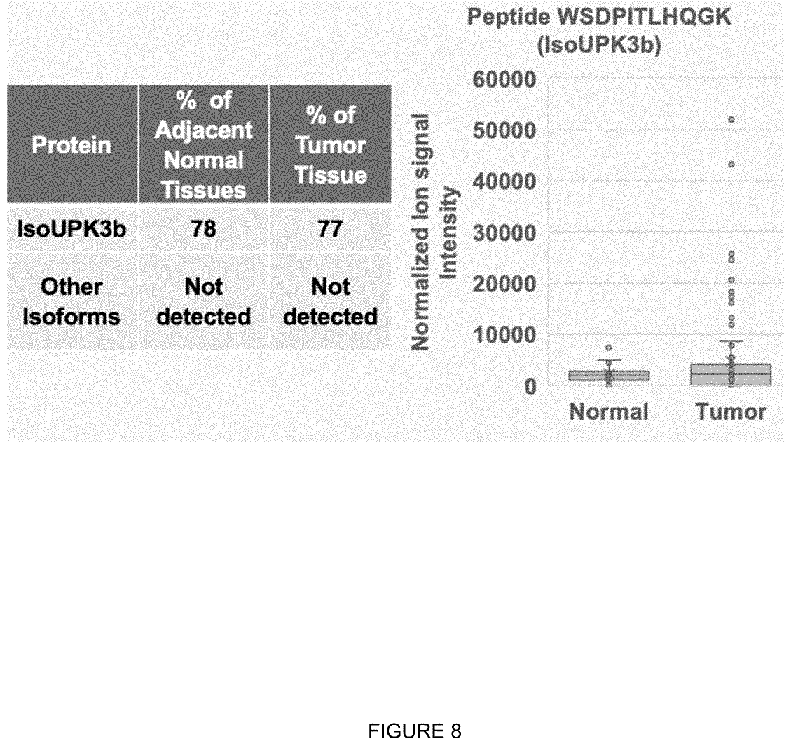

In order to confirm the presence of the UPK3B isoform in ovarian cancer specimens, publicly available proteomic datasets were analyzed to detect the presence of the unique peptide VKFLLMDTRGSPRAETKWS-DPITLHQGKTPGSIDTWPGRRSGSMIVITSILSSLAGLLLLAFLA ASTMRFSSLVWVPEEAPEQLRIGSFMGKRYMTHHIPPSEAATLPVGCKPGLDPLPSLSP (SEQ ID NO:49) and/or a fragment of the unique peptide (WSDPITLHQGK (SEQ ID NO:27)). WSDPITLHQGK (SEQ ID NO:27) is a fragment or sub-peptide of the unique peptide (derived by trypsin processing of the full length protein), where the amount of the sub-peptide is directly proportional to that of the isoform-unique peptide. The sub-peptide may be targeted by a binding molecule (e.g., an antibody). Data mining was performed on 13 datasets originated from the analysis of 94 ovarian tumors and 23 adjacent normal tissue samples with a TMT10plex high-resolution accurate-mass ORBITRAP system. Results show that, although the IsoUPK3B peptide was detected in 77% of tumor samples and 78% of adjacent normal tissues (FIG. 8), its levels were significantly higher in the tumor group (FIG. 8).
B-Cell Lymphomas

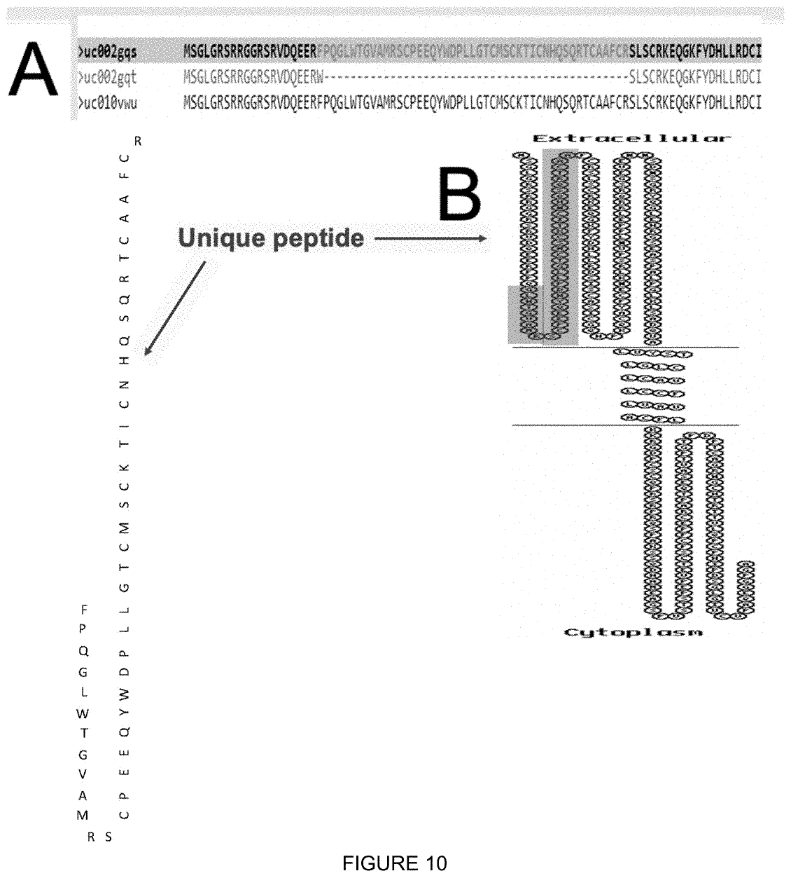

TNF Receptor Superfamily Member 13B (TNFRSF13B) splice variant uc002gqs (ENST00000261652) was analyzed. FIG. 9 shows differential expression results between normal tissues (GTEx database) versus cancer samples (TOGA database). As shown in FIG. 10, the TNFRSF13B isoform identified by the technology described herein has a unique amino acid sequence with a predicted location in the extracellular domain of the protein.

The following is an alignment between the tumor-associated isoform (top; bold; SEQ ID NO:29 (unique sequence underlined)), the canonical form (middle; SEQ ID NO:30), and a minor isoform (bottom; SEQ ID NO:31):

```
>uc002gqs
(SEQ ID NO: 29)
            MSGLGRSRRGGRSRVDQEERFPQGLWTGVAMRSCPEEQYWDPLLGTCMSCKTICNHQSQR
TCAAFCRSLSCREEQGKFYDHLLRDCISCASICGQHPKQCAYFCENKLRSPVNLPPELRRQRSGEV
ENNSDNSGRYQGLEHRGSEASPALPGLKLSADQVALVYSTLGLCLCAVLCCFLVAVACFLKKRGDP
CSCQPRSRPRQSPAKSSQDHAMEAGSPVSTSPEPVETCSFCFPECRAPTQESAVTPGTPDPTCAGR
WGCHTRTTVLQPCPHIPDSGLGIVCVPAQEGGPGA
>uc002gqt
(SEQ ID NO: 30)
                        MSGLGRSRRGGRSRVDQEERW---------------------------------
-------------
SLSCRKEQGKFYDHLLRDCISCASICGQHPKQCAYFCENKLRSPVNLPPELRRQRSGEVENNSDNS
GRYQGLEHRGSEASPALPGLKLSADQVALVYSTLGLCLCAVLCCFLVAVACFLKKRGDPCSCQPRS
RPRQSPAKSSQDHAMEAGSPVSTSPEPVETCSFCFPECRAPTQESAVTPGTPDPTCAGRWGCHTRT
TVLQPCPHIPDSGLGIVCVPAQEGGPGA
>uc010vwu
(SEQ ID NO: 31)
            MSGLGRSRRGGRSRVDQEERFPQGLWTGVAMRSCPEEQYWDPLLGTCMSCKTICNHQSQR
TCAAFCRSLSCRKEQGKFYDHLLRDCISCASICGQHPKQCAYFCENKLRSPVNLPPELRRQRSGEV
ENNSDNSGRYQGLEHRGSEASPA-------------------------------------------
--------PRGCPA-----------------------------------------PGTRKSF----
WDKE----------NFQGEGFHLG-----------
```

The tumor isoform has the following additional amino acid sequence:

(SEQ ID NO: 32)
FPQGLWTGVAMRSCPEEQYWDPLLGTOMSCKTICNHQSQRTCAAFCR.

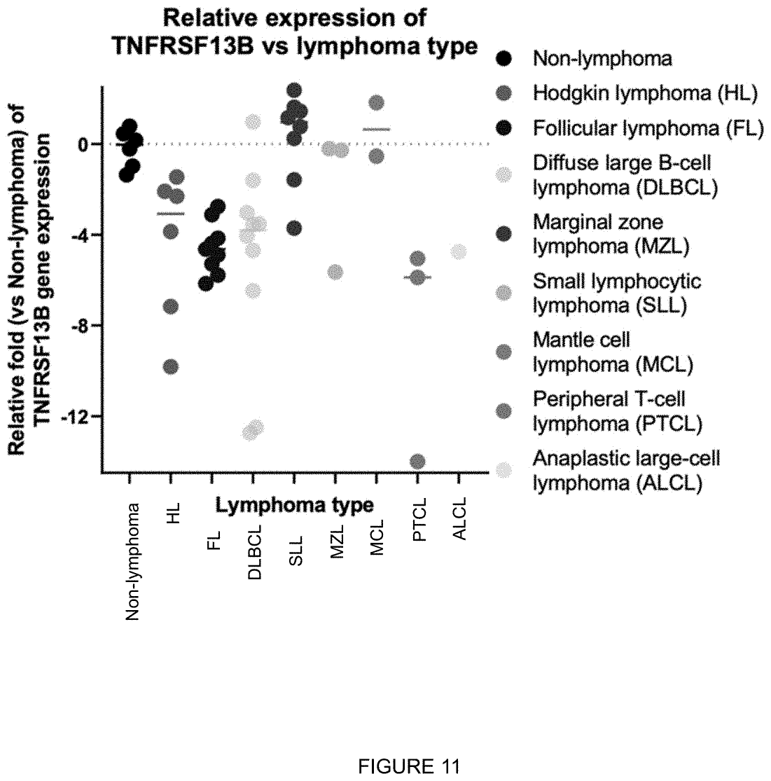

The differential expression of the TNFRSF13B isoform was confirmed by quantitative RT-PCR (qRT-PCR) on a panel of lymphoma samples. FIG. 11 shows the relative fold of expression in a cDNA array of different lymphoma and non-tumor B-cells (detailed in the table below, the isoform of TNFRSF13B was expressed in marginal zone lymphomas (MZL) at higher levels compared with healthy cells, indicating that the TNFRSF13B isoform may be a novel target for MZL. The table below provides a list and quantities of the samples tested in the cDNA array.

| Composition of the cDNA array | |
|---|---|
| Tissue type | Number |
| Non-lymphoma | 6 |
| Hodgkin lymphoma (HL) HL | 6 |
| Follicular lymphoma (FL) | 9 |
| Diffuse large B-cell lymphoma (DLBCL) | 10 |
| Marginal zone lymphoma (MZL) | 8 |
| Small lymphocytic lymphoma (SLL) | 3 |
| Mantle cell lymphoma (MCL) | 2 |
| Peripheral T-cell lymphoma (PTCL) | 3 |
| Anaplastic large-cell lymphoma (ALCL) | 1 |

Identification of MHC-Class I Binding Peptides within a Protein Sequence

Given a selected mutated or cancer splicing derived-antigen, the technology described herein generates all possible peptides of a given pre-selected length containing the mutated amino acid. Such individual target antigen may be queried for peptide-MHC binding affinity. For MHC I, ensembles of allele-specific neural networks are trained to predict peptide-MHC binding affinity. The training dataset is composed of peptide-MHC I binding affinity data from the IEDB database in addition to data from Kim et al., Dataset Size and Composition Impact the Reliability of Performance Benchmarks for peptide-MHC Binding Predictions. BMC Bioinformatics. 2014 Jul. 14;15(1):241. Peptides with lengths between 8-15mer were selected for inclusion in the training set. The following alleles were included in the dataset: HLA-A*01:01, HLA-A*02:02, HLA-A*02:05, HLA-A*02:07, HLA-A*02:12, HLA-A*02:50, HLA-A*11:01, HLA-A*24:02, HLA-A*25:01, HLA-A*26:02, HLA-A*30:02, HLA-A*66:01, HLA-A*68:02, HLA-B*07:02, HLA-B*08:01, HLA-B*51:01, HLA-B*18:01, HLA-B*27:05, HLA-B*39:01, HLA-B*40:01, HLA-B*58:01, and HLA-B*15:01.

Once a target is elected, all possible peptide combinations of the selected length are generated for prediction. Peptides are next encoded into a 15-string placeholder. The first four and last four peptides are always mapped to the first and last four positions to preserve positionality of the stabilizing contacts with the MHC molecules (O'Donnell, T. J. et al. MHCflurry: Open-Source Class I MHC Binding Affinity Prediction. Cell Syst 7, 129-132 e124 (2018)). Peptide representations are then one-hot encoded with 20 features to represent the possible amino acids in a peptide sequence.

Neural Network Architecture and Training (MAAM and MFLM)

The neural network predictors are based on state-of-the-art Capsule Networks (Sabour S, Frosst N, Hinton G. Dynamic Routing Between Capsules. arXiv (2017) 31st Conference on Neural Information Processing Systems (NIPS 2017), Long Beach, Calif., USA). Each network is comprised of three Capsule layers, each of which contains a nested subnetwork. Within a capsule, the network represents features of the data via locally connected one dimensional convolutional layers, without weight sharing. The output from each capsule represents the probability of existence of sub-components of the input peptide. Higher order capsules are dynamically activated via a routing-by-agreement mechanism, similar to the max-pooling operation in a standard convolutional neural network with additional robustness to positional variation.

Nonlinearities are applied to a nested set of layers (the capsule) rather than the output from each individual layer. The output from the capsule network is then fed into a dense feedforward network with scaled exponential linear unit activation (Klambauer G, Unterthiner T, Mayr A. Self-Normalizing Neural Networks. arXiv (2017) 31st Conference on Neural Information Processing Systems (NIPS 2017), Long Beach, Calif., USA). A final linear output unit outputs the nanomolar affinity, transformed between 0.0 and 1.0 via a logarithmic transform (1−log 5000(affinity)). An ensemble of 5 models was trained for each allele, each on a randomly selected subset (80%) of the original data, with 20% used for validation. Models were trained using Adam stochastic optimization (Kingma D, Ba J. Adam: A Method for Stochastic Optimization. arXiv (2017) Published as a conference paper at the 3rd International Conference for Learning Representations, San Diego, 2015) until mean squared error loss reached a minimum value.

Identification of MHC-Class II Binding Peptides within a Protein Sequence (MAAM and MFLM Implementation)

Given a selected mutated antigen or tumor-associated isoform, the technology described herein utilizes the IEDB MHC class II binding prediction tool using the NetMHCIIPan prediction method (Andreatta, M. et al. Accurate pan-specific prediction of peptide-MHC class II binding affinity with improved binding core identification. Immunogenetics 67, 641-650 (2015)) to identify all possible 15-mers peptides binding to a selected HLA class II allele containing the mutated amino acid or at least one amino acid residue present in the tumor-associated isoform but not in the canonical sequence. The following alleles were included in the dataset: DRB1*03:01, DRB1*07:01, DRB1*15:01, DRB1*01:01, DRB1*04:01, DRB1*04:04, DRB1*04:05, DRB1*08:02, and DRB1*09:01.

Identification of Antibody-Bound Peptides within a Protein Sequence (BEM Implementation)

Given a selected mutated antigen or tumor-associated isoform, a BEM can be utilized, or other available module (e.g., BepiPred-2.0 (Jespersen, M. C., Peters, B., Nielsen, M. & Marcatili, P. BepiPred-2.0: improving sequence-based B-cell epitope prediction using conformational epitopes. Nucleic acids research 45, W24-w29 (2017))) to identify all possible linear B-cell epitopes containing the mutated amino acid or at least one amino acid residue present in the tumor-associated isoform but not in the canonical sequence.

Identification of TCR-Activating Peptides within a Protein Sequence (TIM Implementation)

After having determined the MHC binder peptides for class-I molecules, the technology described herein filters the peptides having a predicted affinity of 500 nM or lower to detect those that are more likely to induce a T-cell response by being bound by a T-cell Receptor (TCR). This function is performed by a TIM or REpitope module.

After peptides are identified based on the AI-driven prediction of MHC binding (i.e., from a MAAM and/or MFLM), the REpitope package or a TIM is implemented to predict the targets that are likely to trigger T cell activation in a large portion of the population. REpitope and a TIM utilize a machine learning algorithm that uses amino acid pairwise contact potential matrices as features to evaluate T cell receptor-epitope interaction. The Immune Epitope Database (IEDB) and T cell receptor fragment library are used with selected features to train the algorithm to predict immunogenicity for MHC-I and MHC-II epitopes. An IBS takes sections of the cancer specific peptides and determines the strongest candidates for therapy development using a TIM or the REpitope immunogenicity score.

Immunogenicity score performance can be determined using a suitable process. A performance evaluation can be performed as follows: (1) navigate to https://www.iedb.org/home_v3.php; (2) set search criteria as follows: epitope “linear”, host “human”, assay “T cell assays”, MHC restriction “class I”, disease “any disease;” (3) click on “search;” (4) click on the “assays” tab; (5) select “export results” from the to-right corner of the table, choose “CVS file;” (6) apply a filter for the “Qualitative Measure” column, selecting “positive”, “positive-high”, “positive-intermediate”, and “positive-low;” (7) apply a filter for the “Allele Name” column, de-selecting “HLA-I;” (8) for each epitope, download the FASTA sequence of the corresponding sequence form UniProt; (9) using the “custom sequence” entry mode, copy the full protein sequence from UniProt into the Diamond interface and run the “MHC binding” prediction (or the “long peptides” prediction) using a MAAM or MFLM, respectively; the HLA allele selected for this prediction should be the same HLA allele listed in the Excel database for that specific epitope (from step 8); (10) make a list of all peptides with IC50 less-than-or-equal-to 500 nM; (11) using a TIM, calculate an immunogenicity score for each peptides from step 10; (12) rank the peptides basing on their immunogenicity scores, in descending order; (13) compare the epitope sequence listed in the Excel database with the sequence of the top 20% ranking peptides from the TIM analysis (i.e., steps 11 and 12); and (14) calculate the results: for positive assays, if the top-20% ranking predicted epitopes contain the peptide listed in the Excel database (i.e. the experimentally validated epitope) that is considered a positive (successful) prediction, and for negative assays, if the top-20% ranking predicted epitopes do not contain the peptide listed in the Excel database (i.e. the experimentally validated epitope) that is considered a positive (successful) prediction.

### Example 2: Examples of Implementations

Following is a listing of certain non-limiting implementations of the technology.

A1. A method for computing a binding affinity of an amino acid subsequence for a selected MHC allele or MHC supertype, comprising:
(a) processing an input amino acid sequence into amino acid subsequences of a particular length;
(b) encoding the amino acid subsequences into numerical strings; and
(c) computing a binding affinity value for each of the amino acid subsequences for a MHC allele or MHC supertype from the numerical strings according to bias values and weight values associated with the MHC allele or the MHC supertype, thereby computing binding affinities for the amino acid subsequences for the MHC allele or the MHC supertype, wherein: the computing is performed by a convolutional neural network (CNN) that contains a plurality of virtual neurons arranged in capsules.

A2. The method of embodiment A1, wherein (a) comprises processing the input amino acid sequence into all possible consecutive amino acid subsequences of a selected length n.

A3. The method of embodiment A2, wherein n is an integer between 4 and 20 and output of (a) is a plurality of amino acid subsequences of length n for a corresponding input amino acid sequence.

A4. The method of any one of embodiments A1-A3, wherein (b) comprises integer coding followed by one-hot coding.

A5. The method of embodiment A4, wherein output of (b) is an encoded string of values derived for each counterpart amino acid subsequence.

A6. The method of any one of embodiments A1-A5, wherein each of the capsules is a group of virtual neurons and the capsules are arranged in layers in the CNN.

A7. The method of embodiment A6, wherein there are three layers of capsules.

A8. The method of embodiment A6 or A7, wherein one of the layers is an input layer that receives the strings and passes them to a next layer.

A9. The method of any one of embodiments, A6-A8, wherein one of the layers is a hidden layer that applies transformations to the strings.

A10. The method of embodiment A9, wherein the weight values and the bias values are applied to the strings in the virtual neurons of the hidden layer.

A11. The method of any one of embodiments A6-A10, wherein one of the layers is an output layer.

A12. The method of embodiment A11, wherein the output layer adjusts the strings from the hidden layers to produce each binding affinity.

A13. The method of any one of embodiments A1-A12, wherein:
the weight values and the bias values have been generated from a training process, and
the training process comprises instructing the CNN to process a training dataset using multiple models.

A14. The method of embodiment A13, wherein each of the models is a collection of operations followed until mean-squared error loss reaches a minimum value.

A15. The method of embodiment A13 or A14, wherein five models have been trained for each MHC allele and each MHC supertype.

A16. The method of any one of embodiments A13-A15, wherein:
each of the models has been trained on a randomly selected subset of the dataset, and
a balance of the dataset is utilized for validation.

A17. The method of embodiment A16, wherein 80% of the dataset has been utilized for training the models and 20% of the dataset has been utilized for validation.

A18. The method of any one of embodiments A13-A17, wherein:
the training process comprises implementing an optimization algorithm to train the models, and
the optimization algorithm minimizes output error.

A19. The method of embodiment A18, wherein the optimization algorithm is an Adaptive Moment Estimation (Adam) optimization algorithm or a Stochastic Gradient Descent (SGD) optimization algorithm.

A20. The method of any one of embodiments A13-A19, wherein:
the dataset comprises measured IC50 values; and
the IC50 values are for peptides binding to MHC alleles and/or MHC supertypes.

A21. The method of embodiment A20, wherein the IC50 values are in nanomolar units.

A22. The method of embodiment A20, wherein the IC50 values are transformed to unitless values prior to training.

A23. The method of embodiment A22, wherein the IC50 values are logarithmically transformed.

A24. The method of any one of embodiments A13-A23, wherein one or more of the amino acid subsequences processed in (a) are not present in the dataset.

A25. The method of any one of embodiments A1-A24, wherein:
a single MHC allele is selected, and
a binding affinity for each of the amino acid subsequences processed in (a) is computed in (c) for the single MHC allele selected.

A26. The method of any one of embodiments A1-A24, wherein:
a MHC supertype is selected, and
a binding affinity for each of the amino acid subsequences processed in (a) is computed in (c) for the MHC supertype selected.

A27. The method of any one of embodiments A1-A24, wherein:
a plurality of MHC alleles is selected, and
a binding affinity for each of the amino acid subsequences processed in (a) is computed in (c) for each MHC allele in the plurality of the MHC alleles selected.

A28. The method of any one of embodiments A1-A27, wherein each binding affinity value computed in (c) is a molar binding affinity value.

A29. The method of embodiment A28, wherein the molar binding affinity value is a nanomolar binding affinity value.

A30. The method of embodiment A29 or A29, wherein the molar binding affinity value is transformed into a normalized value.

A31. The method of embodiment A30, wherein the molar binding affinity value is transformed by a logarithmic transformation.

A32. The method of embodiment A31, wherein the logarithmic transformation is according to (1−log 5000(affinity)).

A33. The method of any one of embodiments A28-A32, wherein each binding affinity value computed in (c) is an IC50 value.

A34. The method of any one of embodiments A1-A33, comprising assigning an offset value for each of the amino acid subsequences.

A35. The method of any one of embodiments A1-A34, comprising outputting a list that associates each amino acid subsequence with a binding affinity value computed in (c).

A36. The method of embodiment A35, wherein the list contains only amino acid subsequences associated with a binding affinity value less than, less-than-or-equal-to, greater than, or greater-than-or-equal-to a threshold.

A37. The method of embodiment A35 or A36, wherein the list outputted comprises, for each of the amino acid subsequences listed, two or more of: MHC allele designation or MHC supertype designation, associated MHC binding affinity value, normalized MHC binding affinity value, gene identifier associated with the longer amino acid sequence (e.g., gene name), offset value, index value, and subjective binding affinity descriptor (e.g., strong, intermediate).

A38. The method of embodiment A37, wherein the subjective binding descriptor is assigned according to a binding affinity value threshold.

A39. The method of embodiment A38, wherein:
a strong subjective binding affinity descriptor is assigned for an amino acid subsequence associated with a binding affinity value that is less than 500 nM,
an intermediate subjective binding affinity descriptor is assigned for an amino acid subsequence associated with a binding affinity value that is between 500 nanomolar and 1,000 nanomolar, and
a weak subjective binding affinity descriptor is assigned for an amino acid subsequence associated with a binding affinity that is greater than 1,000 nanomolar.

A40. The method of any one of embodiments A1-A39, wherein:
the MHC allele is a MHC class I allele or a MHC class II allele, and/or
the MHC supertype is a MHC class I supertype or a MHC class II supertype.

A41. The method of embodiment A40, wherein:
the MHC class I allele is a HLA class I allele; and/or
the MHC class I supertype is a HLA class I supertype.

A42. The method of embodiment A41, wherein:
the HLA class I allele is a HLA-A allele or a HLA-B allele; and/or
the HLA class I supertype is a HLA-A supertype or HLA-B supertype.

A43. The method of embodiment A42, wherein:
the HLA-A allele is a A1, A2, A3, A24 or A26 allele; and/or
the HLA-A supertype is a A1, A2, A3, A24 or A26 supertype.

A44. The method of embodiment A42, wherein:
the HLA-B allele is a B7, B8, B27, B39, B44, B58 or B62 allele; and/or
the HLA-B supertype is a B7, B8, B27, B39, B44, B58 or B62 supertype.

A45. The method of any one of embodiments A42-A44, wherein:
the HLA-A allele is chosen from HLA-A*01:01, HLA-A*02:02, HLA-A*02:05, HLA-A*02:07, HLA-A*02:12, HLA-A*02:50, HLA-A*11:01, HLA-A*24:02, HLA-A*25:01, HLA-A*26:02, HLA-A*30:02, HLA-A*66:01 and HLA-A*68:02, and/or
the HLA-B allele is chosen from HLA-B*07:02, HLA-B*08:01, HLA-B*51:01, HLA-B*18:01, HLA-B*27:05 HLA-B*39:01, HLA-B*40:01, HLA-B*58:01 and HLA-B*15:01.

A46. The method of any one of embodiments A40-A45, wherein:
the MHC class II allele is a HLA class II allele; and/or
the MHC class II supertype is a HLA class II supertype.

A47. The method of embodiment A46, wherein:
the HLA class II allele is a DR, DQ or DP allele; and/or
the HLA class II supertype is a DR, DQ or DP supertype.

A48. The method of embodiment A47, wherein:
the DR allele is a DR1, DR3, DR4, DR5 or DR9 allele, or is an allele within a supertype chosen from four supertypes: (DRB1*0401, DRB1*0405, DRB1*0802, DRB1*1101), (DRB3*0101, DRB3*0202), (DRB1*0301, DRB1*1302), and the fourth containing the remaining DR proteins; and/or
the DR supertype is a DR1, DR3, DR4, DR5 or DR9 supertype, or is chosen from four supertypes: (DRB1*0401, DRB1*0405, DRB1*0802, DRB1*1101), (DRB3*0101, DRB3*0202), (DRB1*0301, DRB1*1302), and the fourth containing the remaining DR proteins.

A49. The method of embodiment A47, wherein:
the DQ allele is a DQ1, DQ2, DQ3 allele, or is an allele within a supertype chosen from two supertypes: (DQB1*0301, DQB1*0302, DQB1*0401) and (DQB1*0201, DQB1*0501, DQB1*0602); and/or
the DQ supertype is a DQ1, DQ2, DQ3 supertype, or is chosen from two supertypes: (DQB1*0301, DQB1*0302, DQB1*0401) and (DQB1*0201, DQB1*0501, DQB1*0602).

A50. The method of embodiment A47, wherein:
the DP allele is a DPw1, DPw2, DPw4 or DPw6 supertype, or is an allele within the supertype (DPB1*0101, DPB1*0201, DPB1*0401, DPB1*0402, DPB1*0501, and DPB1*1401); and/or
the DP supertype is a DPw1, DPw2, DPw4 or DPw6 supertype, or is chosen from the supertype (DPB1*0101, DPB1*0201, DPB1*0401, DPB1*0402, DPB1*0501, and DPB1*1401).

A51. The method of embodiment A47, wherein the HLA class II allele is chosen from DRB1*03:01, DRB1*07:01, DRB1*15:01, DRB1*01:01, DRB1*04:01, DRB1*04:04, DRB1*04:05, DRB1*08:02 and DRB1*09:01.

B1. The method of any one of embodiments A1-A51, comprising mapping each amino acid subsequence for which the binding affinity is computed to an amino acid sequence that contains the amino acid subsequence.

B2. The method of embodiment B1, wherein the mapping comprises identifying a subsequence within the longer amino acid sequence having an exact match to each amino acid subsequence for which for which the binding affinity is computed.

B3. The method of embodiment B1 or B2, wherein an offset value for each of the mapped amino acid subsequences is determined.

B4. The method of any one of embodiments B1-B3, wherein the amino acid subsequences mapped are limited to a subset of amino acid subsequences having a MHC binding affinity value computed that is less than, less-than-or-equal-to, greater than, or greater-than-or-equal-to, a binding affinity threshold.

B5. The method of any one of embodiments B1-B4, comprising outputting a graphic representation of the amino acid sequence and mapped amino acid subsequences.

B6. The method of embodiment B5, wherein the graphic representation includes:
a color rendition of a region in the amino acid sequence in which there are overlapping amino acid subsequences mapped is different than the color rendition for a region in which only one amino acid subsequence is mapped or to which no amino subsequences is mapped; and/or
a color rendition of region in the amino acid sequence in which there are two overlapping amino acid subsequences mapped is different than a color rendition of a region in which there are three overlapping amino acid subsequences.

B7. The method of embodiment B5 or B6, wherein the graphic representation is associated with a binding affinity value for each amino acid subsequence mapped to the amino acid sequence.

B8. The method of any one of embodiments B5-B7, wherein:
a list is outputted; and
the list comprises for each of the amino acid subsequences mapped to the amino acid sequence in the graphic representation, two or more of: MHC allele designation or MHC supertype designation, associated binding affinity value, normalized binding affinity value, gene identifier associated with the amino acid sequence, offset value, index value, and subjective binding affinity descriptor.

C1. A method for computing or receiving a composite binding affinity value of an amino acid subsequence for a selected MHC allele or MHC supertype, comprising: for an input amino acid sequence processed into amino acid subsequences of a particular length, computing or receiving a composite binding affinity value for each of the amino acid subsequences based on (i) a proteasome cleavage score for the amino acid subsequence, and/or (ii) a transporter affinity score for the amino acid subsequence, and/or (iii) a MHC allele or MHC supertype binding affinity value for the amino acid subsequence.

C2. The method of embodiment C1, wherein the proteasome cleavage score is a value based on a likelihood of proteasome processing an input amino acid sequence into an amino acid subsequence processed in (a).

C3. The method of embodiment C1 or C2, wherein the transporter affinity score is a value based on a likelihood of a particular amino acid subsequence binding a transporter protein.

C4. The method of embodiment C3, wherein:
the transporter protein is a transporter associated with antigen processing (TAP), and
the transporter affinity score is a TAP affinity score.

C5. The method of any one of embodiments C1-04, wherein the MHC allele or the MHC supertype binding affinity value for the amino acid subsequence are computed according to a method of any one of embodiments A1-A51 and/or B1-B8.

C6. The method of any one of embodiments C1-05, wherein the (i) proteasome cleavage score, and/or (ii) the transporter affinity score, and/or (iii) a MHC allele or MHC supertype binding affinity value, for each amino acid subsequence, independently is received or is computed.

C7. The method of any one of embodiments C1-06, wherein the MHC allele or the MHC supertype binding affinity value is a normalized binding affinity value.

C8. The method of embodiment C7, wherein the normalized binding affinity value normalized according to a percentile.

C9. The method of embodiment C8, wherein the percentile is a first percentile.

C10. The method of any one of embodiments C7-C9, wherein the composite value equals (normalized binding affinity value)+((0.1)*(proteasome cleavage score)) ((0.05)*(TAP affinity score)).

C11. The method of any one of embodiments C1-010, comprising outputting a list comprising, for each of the amino acid subsequences listed, two or more of: MHC allele designation or MHC supertype designation, associated composite value, associated MHC binding affinity value, normalized binding affinity value, proteasome cleavage score, transporter affinity score (e.g., TAP affinity score), gene identifier associated with the longer amino acid sequence, offset value, index value, and subjective binding affinity descriptor (e.g., strong, intermediate).

C12. The method of any one of embodiments C1-011, comprising outputting a graphic representation of the amino acid sequence and mapped amino acid subsequences.

C13. The method of embodiment C12, wherein the graphic representation includes:
a color rendition of a region in the amino acid sequence in which there are overlapping amino acid subsequences mapped is different than the color rendition for a region in which only one amino acid subsequence is mapped or to which no amino subsequences is mapped; and/or
a color rendition of region in the amino acid sequence in which there are two overlapping amino acid subsequences mapped is different than a color rendition of a region in which there are three overlapping amino acid subsequences.

D1. A method for computing a T-cell receptor (TCR) immunogenicity score, comprising computing, for each amino acid subsequence in a plurality of amino acid subsequences of one or more amino acid sequences, a TCR immunogenicity score according to a TCR-peptide contact potential profiling (CPP) process.

D2. The method of embodiment D1, wherein the immunogenicity score is computed according to (i) the TCR-peptide contact potential profiling (CPP) assessment, and (ii) one or more of: target peptide length, amino acid in each position of the amino acid subsequence, and descriptors for the amino acid subsequence.

D3. The method of embodiment D1 or D2, wherein the amino acid subsequences:
are the amino acid subsequences of a method of any one of embodiments A1-A51, B1-B8 and/or C1-013, whereby the amino acid subsequences are target fragment sequences; and/or
are target fragment sequences generated by processing the amino acid subsequences of a method of any one of embodiments A1-A51, B1-B8 and/or C1-013 into amino acid subsequences of a particular length; and/or
are target fragment sequences generated by processing an amino acid sequence from which the amino acid subsequences of a method of any one of embodiments A1-A51, B1-B8 and/or C1-013 were generated, into amino acid subsequences of a particular length.

D4. The method of any one of embodiments D1-D3, wherein the immunogenicity score is computed according to amino acid pairwise contact potential matrices from the CPP.

D5. The method of embodiment D3 or D4, comprising:
(i) optionally generating the target fragment sequences;
(ii) generating an alignment pair between each target fragment sequence and a TCR fragment sequence from a plurality of TCR fragment sequences, and
(iii) computing an alignment score for each alignment pair.

D6. The method of embodiment D5, comprising generating an optimized alignment between the target fragment sequence and the TCR fragment sequence in each alignment pair, where the optimized alignment maximizes the alignment score for the alignment pair.

D7. The method of embodiment D5 or D6, wherein:
computing the alignment score comprises generating a sum of pairwise scores, and
each of the pairwise scores is a score generated for an amino acid in the target fragment sequence and an aligned amino acid in the TCR fragment sequence for the alignment pair.

D8. The method of any one of embodiments D5-D7, wherein there are no gaps between the target fragment sequence and the TCR fragment sequence in each alignment pair.

D9. The method of any one of embodiments D5-D8, wherein the target fragment sequence and/or the TCR fragment sequence in each alignment pair is about 3 amino acids to about 8 amino acids in length.

D10. The method of any one of embodiments D5-D8, wherein the target fragment sequence and/or the TCR fragment sequence in each alignment pair is about 3 amino acids to about 11 amino acids in length.

D11. The method of any one of embodiments D5-D10, comprising assembling the alignment scores into, or fitting the alignment scores to, amino acid pairwise contact potential matrices.

D12. The method of any one of embodiments D5-D11, wherein a portion of the TCR fragment sequences is, or the TCR fragment sequences are, TCR CDR3 beta-chain fragment sequences.

D13. The method of embodiment D12, wherein the TCR CDR3 beta-chain fragment sequences are a portion of amino acid subsequences of TCRs attributed to a CDR3 of a beta-chain of a TCR, or a portion thereof.

D14. The method of embodiment D12 or D13, wherein the TCR CDR3 beta-chain fragment sequences include (i) sliding window-generated fragment sequences, and/or (ii) portions of reverse-oriented TCR CDR3 beta-chain sequences, and/or (iii) TCR CDR3 beta-chain fragment sequences from CD4+ T-cells, and/or (iv) TCR CDR3 beta-chain fragment sequences from CD8+ T-cells.

D15. The method of any one of embodiments D1-D14, comprising outputting a list of amino acid subsequences and associated immunogenicity scores.

E1. A method for identifying a disease-associated polypeptide variant, comprising:
for a dataset containing expression level values for transcripts in disease samples and non-disease samples from multiple tissues, wherein the dataset includes transcripts corresponding to amino acid sequence variants encoded by a gene,
(a) computing an average expression level value for each transcript for disease samples;
(b) computing for each amino acid sequence variant encoded by a gene a related variant value for disease samples and a related variant value for non-disease samples, wherein the related variant value is (i) an average expression level value for the variant, divided by (ii) a sum of average expression level values for each variant of the gene; and
(c) computing for each amino acid sequence variant a fold change value, where the fold change value is (i) the average expression level value for the amino acid sequence variant in disease samples, divided by (ii) the average expression level value for the amino acid sequence variant in non-disease samples.

E2. The method of embodiment E1, wherein the related variant value is a percentage.

E3. The method of embodiment E1 or E2, comprising in part (a) matching each average expression level value for each transcript with (i) a composite average expression level for the transcript for all non-disease samples for each tissue, and/or (ii) a highest tissue expression level identified from all non-disease samples for the transcript.

E4. The method of any one of embodiments E1-E3, comprising, after performing (a) and/or (b) and/or (c), outputting a disease sample only variant list, wherein each variant selected for the list is expressed in disease samples and insignificantly expressed or not expressed in non-diseased samples.

E5. The method of embodiment E4, wherein the average expression level value is a TPM level and insignificantly expressed is an average expression level value of less than 0.00001.

E6. The method of any one of embodiments E1-E5, comprising, after performing (a) and/or (b) and/or (c), outputting a disease sample specific variant list, wherein each variant selected for the disease sample specific variant list is (i) the dominant variant in disease samples, and/or (ii) not the dominant variant in non-disease samples.

E7. The method of any one of embodiments E1-E6, comprising, after performing (a) and/or (b) and/or (c), outputting a disease upregulated variant list, wherein each variant selected for the list exhibits a fold change value great than or greater-than-or-equal-to a threshold value.

E8. The method of embodiment E7, wherein the threshold value is a two-fold threshold value.

E9. The method of any one of embodiments E4-E8, comprising, after performing (a) and/or (b) and/or (c), outputting a multi-sequence alignment (MSA) for each variant included in a list.

E10. The method of any one of embodiments E1-E9, comprising, after performing (a) and/or (b) and/or (c), outputting a box plot of average expression level values for non-disease samples by tissue for a given variant.

E11. The method of any one of embodiments E1-E10, comprising, after performing (a) and/or (b) and/or (c), outputting a box plot of expression level values of a variant in disease samples for different tissues.

E12. The method of embodiment E11, comprising displaying one or more of: an upper whisker, a lower whisker, an upper quartile, a lower quartile, an average of the distribution of values for a selected disease type, and a maximum expression level value for a non-disease sample from a tissue.

E13. The method of any one of embodiments E1-E12, wherein a gene of interest is selected and output is limited to amino acid sequence variants for the gene for multiple tissues.

E14. The method of any one of embodiments E1-E12, wherein a tissue of interest is selected and output includes amino acid sequence variants for multiple genes for the tissue.

E15. The method of any one of embodiments E1-E14, wherein an expression level value threshold filter is selected and output is limited to amino acid sequence variants associated with a minimum disease sample expression level value.

E16. The method of any one of embodiments E1-E15, wherein an expression level value threshold filter is selected that limits output to variants associated with a maximum non-disease sample expression level value.

E17. The method of any one of embodiments E1-E16, wherein a filter is selected that limits output to amino acid sequence variants of genes that encode a cell-surface polypeptide.

E18. The method of any one of embodiments E1-E17, wherein a filter is selected that limits output to amino acid sequence variants having at least one insertion of a single amino acid or two or more consecutive amino acids relative to a canonical amino acid sequence.

E19. The method of any one of embodiments E1-E18, wherein an amino acid sequence variant comprises at least one variation relative to another amino acid variant encoded by the same gene chosen from a single amino acid substitution, single amino acid insertion, single amino acid deletion, substitution of two or more consecutive amino acids, insertion of two or more consecutive amino acids, deletion of two or more consecutive amino acids, or a combination thereof.

E20. The method of any one of embodiments E1-E19, wherein expression level values are transcripts per million (TPM) values, fragments per kilobase per million reads mapped (FPKM) values, reads per kilobase per million reads mapped (RPKM) values, RNA-seq by expectation-maximization (RSEM) values, or combination of such values.

E21. The method of any one of embodiments E1-E20, wherein expression level values are average TPM values, average FPKM values, average RPKM values, average RSEM values, or combination thereof.

E22. The method of any one of embodiments E1-E21, wherein an average value is a mean, median or mode value.

E23. The method of any one of embodiments E1-E22, wherein each disease sample in the dataset is associated with (i) a tissue of origin, and (ii) matched expression level values for each transcript derived from non-disease tissue adjacent to the disease tissue.

E24. The method of any one of embodiments E1-E23, wherein a disease sample is a sample from a subject having or suspected of having Alzheimer's disease, Parkinson's disease, Lupus, IPEX syndrome, diabetes, rheumatoid arthritis, influenza, pneumonia, or tuberculosis. A disease sample can be a cancer sample, and non-limiting examples of cancer samples include samples from subjects having or suspected as having acute myeloid leukemia, adrenocortical carcinoma, bladder urothelial carcinoma, brain lower grade glioma, breast invasive carcinoma, cervical squamous cell carcinoma and endocervical adenocarcinoma, cholangiocarcinoma, colon adenocarcinoma, esophageal carcinoma, glioblastoma multiforme, head and neck squamous cell carcinoma, kidney chromophobe, kidney renal clear cell carcinoma, kidney renal papillary cell carcinoma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thymoma, thyroid carcinoma, uterine carcinosarcoma, uterine corpus endometrial carcinoma, uveal melanoma, chronic myeloid leukemia, acute lymphocytic leukemia, multiple myeloma, non-Hodgkin lymphoma, Hodgkin lymphoma, marginal zone lymphoma, follicular lymphoma, small lymphocyte lymphoma, E-cell lymphoma, diffuse large E-cell lymphoma or mantle cell lymphoma.

F1. A method for predicting a location of one or more B-cell receptor (BCR) epitopes in an amino acid sequence, comprising:
generating a BCR epitope score for each amino acid in an input amino acid sequence indicative of the probability that the amino acid exists within a BCR epitope based on a hidden Markov model and a propensity scale process, or a Random Forest Regression (RF) algorithm;
predicting that the amino acid is in a BCR epitope in a polypeptide corresponding to the input amino acid sequence according to a score threshold.

F2. The method of embodiment F1, wherein the BCR epitopes comprise linear BCR epitopes and/or conformational BCR epitopes.

F3. The method of embodiment F1 or F2, wherein the threshold is configurable.

F4. The method of any one of embodiments F1-F3, wherein the scores are not affected by the threshold.

F5. The method of any one of embodiments F1-F4, comprising computing or receiving one or more scores pertaining to (i) likelihood that the amino acid exists in a secondary structure in the corresponding polypeptide; and/or (ii) likelihood that the amino acid is exposed or buried in the corresponding polypeptide.

F6. The method of embodiment F5, wherein the secondary structure is an alpha helix structure or beta sheet structure.

F7. The method of any one of embodiments F1-F6, comprising outputting a graphic representation showing the input amino acid sequence annotated with (i) a BCR epitope score above a threshold displayed adjacent to each corresponding amino acid in the input amino acid sequence; and/or (ii) an indicator as to whether an amino acid in the input sequence is located in a BCR epitope; and/or (iii) one or more color gradients scaled to the BCR epitope scores.

F8. The method of embodiment F7, wherein:
the graphic representation comprises one or more scores pertaining to (i) likelihood that an amino acid exists in a secondary structure, and/or (ii) likelihood that an amino acid is buried or exposed in the corresponding polypeptide; and/or
one or more color gradients scaled to the one or more additional scores.

F9. The method of any one of embodiments F1-F7, wherein the input amino acid sequence is of a disease-associated sequence variant identified by a method of any one of embodiments E1-E24.

G1. A method of any one of embodiments, A1-A51, B1-B8, C1-013, D1-D15, E1-E24 and/or F1-F9, comprising receiving an input amino acid sequence or amino acid subsequence from a sequence acquisition interface (SAD.

G2. The method of embodiment G1, wherein:
a gene identifier is inputted into a SAI, and
an algorithm associated with the SAI identifies the gene identifier in a database and retrieves the associated amino acid sequence from the database.

G3. The method of embodiment G2, wherein the gene identifier is a gene name, a gene tag or an accession number.

G4. The method of any one of embodiments G1-G3, comprising inputting the amino acid sequence directly into the SAI (e.g., copying and pasting an amino acid sequence).

G5. The method of any one of embodiments G1-G4, wherein the input amino acid sequence is a polypeptide amino acid sequence, a peptide amino acid sequence, or a portion of a polypeptide amino acid sequence or a peptide amino acid sequence.

H1. A process, comprising:
(a) implementing a method of any one of embodiments A1-A51, and/or B1-B8, and/or 01-013 in association with receiving one or more input amino acid sequences; and
(b) outputting a subset of amino acid subsequences in the input amino acid sequences according to a MHC binding affinity value threshold, wherein the amino acid subsequences in the subset, and/or longer amino acid sequences each containing one or more of the amino acid subsequences in the subset, are immunogenic candidates.

H2. A process, comprising:
(a) implementing a method of any one of embodiments A1-A51, and/or B1-B8, and/or C1-C13 in association with receiving one or more input amino acid sequences;
(b) outputting a subset of amino acid subsequences in the input amino acid sequences according to a MHC binding affinity value threshold;
(c) implementing a method of any one of embodiments D1-D15 in association with receiving the amino acid subsequences in the subset outputted in (b), and/or longer amino acid sequences each containing one or more of the amino acid subsequences in the subset outputted in (b); and
(d) outputting a subset of the amino acid subsequences and/or amino acid sequences received in (c) according to an immunogenicity score threshold, wherein the amino acid subsequences and/or amino acid sequences in the subset outputted in (d) are immunogenic candidates.

H3. The process of embodiment H1 or embodiment H2, comprising:
(1) implementing a method of any one of embodiments F1-F9 in association with receiving one or more of the input amino acid sequences received in (a);
(2) outputting a subset of amino acid subsequences in the input amino acid sequences according to a B-cell receptor (BCR) score threshold;
(3) comparing (i) the subset of the amino acid subsequences in the subset of (2), and/or one or more amino acid sequences each containing one or more of the amino acid subsequences in the subset of (2), with (ii) the amino acid subsequences in the subset of (b) in H1, and/or longer amino acid sequences each containing one or more of the amino acid subsequences in the subset of (b) in H1, and/or with (iii) the amino acid subsequences in the subset of (d) in H2, and/or longer amino acid sequences each containing one or more of the amino acid subsequences in the subset of (d) in H2; wherein:
amino acid subsequences in the subset of (2), and/or one or more amino acid sequences each containing one or more of the amino acid subsequences in the subset of (2), that are present in (ii) or (iii) are considered immunogenic candidates.

H4. A process of any one of embodiments H1-H3, comprising:
implementing a method of any one of embodiments E1-E24; and
outputting one or more disease-associated amino acid sequence variants, which are the one or more input amino acid sequences of (a) in H1, of (a) in H2 and/or (1) in H3.

H5. A process, comprising:
implementing a method of any one of embodiments E1-E24;
outputting one or more disease-associated amino acid sequence variants;
implementing a method of any one of embodiments F1-F9 in association with receiving the one or more disease-associated amino acid sequence variants as input amino acid sequences; and
outputting a subset of amino acid subsequences in the input amino acid sequences according to a B-cell receptor (BCR) score threshold, wherein the amino acid subsequences in the subset, and/or one or more amino acid sequences each containing one or more of the amino acid subsequences in the subset, are considered immunogenic candidates.

I1. A method, comprising contacting components of a polypeptide interaction system with a polypeptide comprising an amino acid sequence or amino acid subsequence identified by a method of any one of embodiments A1-A51, B1-B8, C1-013, D1-D15, E1-E24, F1-F9 and/or G1-G5.

I2. The method of embodiment I1, wherein the polypeptide interaction system is an in vitro system or ex vivo system.

I3. The method of embodiment I1, wherein the polypeptide interaction system is an in vivo system.

I4. The method of any one of embodiments I1-I3, wherein the polypeptide interaction system comprises antigen presenting cells.

I5. The method of embodiment I4, wherein the antigen presenting cells comprise one or more of the following cell types: dendritic cells, B-cells and macrophages.

I6. The method of embodiment I4 or I5, wherein the antigen presenting cells display the polypeptide or portion thereof.

I7. The method of any one of embodiments I1-I6, wherein the polypeptide interaction system comprises T-cells.

I8. The method of embodiment I7, wherein the T-cells comprise one or more of the following cell types: CD4+ T-cells, CD8+ T-cells and gamma-delta T-cells.

I9. The method of embodiment I7 or I8, wherein the T-cells comprise tumor-infiltrating lymphocytes and/or peripheral blood lymphocytes.

I10. The method of any one of embodiments I7-I9, wherein the T-cells comprise expanded T-cells.

I11. The method of any one of embodiments I7-I10, wherein the T-cells comprise activated T-cells.

I12. The method of any one of embodiments I7-I10, comprising determining T-cell cytotoxicity.

I13. The method of any one of embodiments I1-I12, comprising B-cells.

I14. The method of embodiment I13, wherein the B-cells comprise activated B-cells.

I15. The method of embodiment I13 or I14, comprising determining binding of the B-cells to the polypeptide.

I15.1. The method of any one of embodiments I1-I15, comprising one or more of invariant natural killer T cells (iNKT), NK cells, and mucosal-associated innate T (MAIT) cells.

I16. A method of any one of embodiments A1-A51, B1-B8, C1-013, D1-D15, E1-E24, F1-F9, G1-G5 and/or I1-I15.1, comprising determining presence, absence and/or amount of a polypeptide comprising an immunogenic candidate polypeptide sequence, or portion thereof, in polypeptides of disease samples and/or non-disease samples.

I17. The method of embodiment I16, wherein the polypeptides of disease samples and/or non-disease samples are determined by mass spectrometry.

J1. An isolated polypeptide or a polynucleotide encoding a polypeptide, wherein the polypeptide comprises an amino acid subsequence identified by a method of any one of embodiments A1-A51, B1-B8, C1-013 and/or D1-D15.

J2. The isolated polypeptide or polynucleotide of embodiment J2, wherein the polypeptide comprises the amino acid subsequence of SEQ ID NO:4, SEQ ID NO:20, or SEQ ID NO:27.

J3. An isolated polypeptide or a polynucleotide encoding a polypeptide, wherein the polypeptide comprises an amino acid sequence or a subsequence thereof identified by a method of any one of embodiments E1-E24.

J4. The isolated polypeptide or polynucleotide of embodiment J4, wherein the polypeptide comprises the amino acid subsequence of SEQ ID NO:3, SEQ ID NO:19, SEQ ID NO:32 or SEQ ID NO:49.

J5. The isolated polypeptide or polynucleotide of any one of embodiments J1-J4, wherein the amino acid subsequence or the amino acid sequence is from a polypeptide encoded by a human gene.

J6. The isolated polynucleotide of embodiment J5, comprising a first polynucleotide portion encoding the polypeptide and a second polynucleotide portion from a non-human organism.

J7. The isolated polynucleotide of embodiment J6, wherein the non-human organism is a virus or bacterium.

J8. The isolated polynucleotide of embodiment J6 or J7, wherein the polynucleotide is an expression vector or expression plasmid.

J9. The isolated polynucleotide of embodiment J6 or J7, wherein the polynucleotide is a DNA plasmid or vector, or RNA plasmid or vector.

J10. The isolated polynucleotide of embodiment J9, wherein the second polynucleotide portion of the DNA plasmid or vector comprises a DNA virus or portion thereof.

J11. The isolated polynucleotide of embodiment J10, wherein the DNA virus is a herpesvirus, an adenovirus or a poxvirus.

J12. The isolated polynucleotide of embodiment J9, wherein the second polynucleotide portion of the RNA plasmid or vector comprises a RNA virus.

J13. The isolated polynucleotide of embodiment J12, wherein the RNA virus is a retrovirus or a ssRNA virus.

J14. A vaccine composition comprising an isolated polypeptide or polynucleotide of any one of embodiments J1-J13, and comprising one or more suitable pharmaceutically acceptable adjuvants and/or one or more suitable pharmaceutically acceptable carriers.

J15. A method for treating a condition in a subject, comprising administering a polypeptide, polynucleotide or vaccine of any one of embodiments J1-J14 to a subject in need thereof, in an amount sufficient to induce an immune response against the polypeptide.

J16. A composition comprising an antigen presenting cell (APC) and a polypeptide or polynucleotide of any one of embodiments J1-J13.

J17. The composition of embodiment J16, wherein the polynucleotide resides within the APC.

J18. The composition of embodiment J16 or J17, wherein the polypeptide or portion thereof is presented on the surface of the APC.

J19. The composition of any one of embodiments J16-J18, wherein the APC is a dendritic cell.

J20. A method for treating a condition in a subject, comprising administering a composition of any one of embodiments J16-J19 to a subject in need thereof, in an amount sufficient to induce an immune response against the polypeptide.

J21. A method of inducing an immune response in a subject, comprising administering a polypeptide, polynucleotide or vaccine of any one of embodiments J1-J13 to a subject in an amount sufficient to induce an immune response against the polypeptide.

J22. The method of embodiment J21, comprising obtaining antiserum from the subject.

J23. The method of embodiment J21 or J22, comprising obtaining polyclonal antibodies from the subject and/or antiserum that immunospecifically bind to the polypeptide.

J24. The method of embodiment J21, comprising:
isolating spleen cells from the subject, and
combining the spleen cells with myeloma cells under conditions that produce monoclonal antibody generating hybridomas.

J25. The method of embodiment J24, comprising screening hybridomas for those that produce monoclonal antibodies that immunospecifically bind to the polypeptide.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A method for computing a binding affinity of an amino acid subsequence for a selected MHC allele or MHC supertype, comprising:
(a) processing an input amino acid sequence into amino acid subsequences of a particular length;
(b) encoding the amino acid subsequences into numerical strings; and
(c) computing a binding affinity value for each of the amino acid subsequences for a MHC allele or MHC supertype from the numerical strings according to bias values and weight values associated with the MHC allele or the MHC supertype, thereby computing binding affinities for the amino acid subsequences for the MHC allele or the MHC supertype, wherein: the computing is performed by a convolutional neural network (CNN) that contains a plurality of virtual neurons arranged in capsules.

2. The method of claim 1, wherein (a) comprises processing the input amino acid sequence into all possible consecutive amino acid subsequences of a selected length n.

3. The method of claim 1, wherein (b) comprises integer coding followed by one-hot coding.

4. The method of claim 1, wherein there are three layers of capsules.

5. The method of claim 1, wherein:
the weight values and the bias values have been generated from a training process, and
the training process comprises instructing the CNN to process a training dataset using multiple models.

6. The method of claim 5, wherein the training process comprises implementing an Adaptive Moment Estimation (Adam) optimization algorithm to train the models.

7. The method of claim 1, comprising outputting a list comprising, for each of the amino acid subsequences listed, two or more of: MHC allele designation or MHC supertype designation, associated MHC binding affinity value, normalized MHC binding affinity value, gene identifier associated with the longer amino acid sequence, offset value, index value, and subjective binding affinity descriptor.

8. The method of claim 1, comprising mapping each amino acid subsequence for which the binding affinity is computed to an amino acid sequence that contains the amino acid subsequence.

9. The method of claim 8, comprising outputting a graphic representation of the amino acid sequence and mapped amino acid subsequences.

10. A method for identifying a disease-associated polypeptide variant, comprising:
for a dataset containing expression level values for transcripts in disease samples and non-disease samples from multiple tissues, wherein the dataset includes transcripts corresponding to amino acid sequence variants encoded by a gene,
(a) computing an average expression level value for each transcript for disease samples;
(b) computing for each amino acid sequence variant encoded by a gene a related variant value for disease samples and a related variant value for non-disease samples, wherein the related variant value is (i) an average expression level value for the variant, divided by (ii) a sum of average expression level values for each variant of the gene; and
(c) computing for each amino acid sequence variant a fold change value, where the fold change value is (i) the average expression level value for the amino acid sequence variant in disease samples, divided by (ii) the average expression level value for the amino acid sequence variant in non-disease samples.

11-29. (canceled)


SEQUENCE LISTING (105 sequences)

SEQ ID NO: 1 - protein, 136 aa, Homo sapiens
MAVILGIFGTVQYRSRYLILDRDFIMTFNTSLHRSWWMENGPGCLVTPVLNSRLALEDHHVISVTGCLLDYPYIEALSSALQIFLALFGFVFACYVSKVFLEEEDSFDFIGGFDSYGYQAPQKTSHLQLQPLYTSG

SEQ ID NO: 2 - protein, 163 aa, Homo sapiens
MAVILGIFGTVQYRSRYLILYAAWLVLWVGWNAFIICFYLEVGQLSQDRDFIMTFNTSLHRSWWMENGPGCLVTPVLNSRLALEDHHVISVTGCLLDYPYIEALSSALQIFLALFGFVFACYVSKVFLEEEDSFDFIGGFDSYGYQAPQKTSHLQLQPLYTSG

SEQ ID NO: 3 - protein, 27 aa, Homo sapiens
YAAWLVLWVGWNAFIICFYLEVGQLSQ

SEQ ID NO: 4 - protein, 26 aa, Homo sapiens
YLILYAAWLVLWVGWNAFIICFYLEV

SEQ ID NO: 5 - protein, 9 aa, Homo sapiens
FIICFYLEV

SEQ ID NO: 6 - protein, 9 aa, Homo sapiens
LYAAWLVLW

SEQ ID NO: 7 - protein, 9 aa, Homo sapiens
WNAFIICFY

SEQ ID NO: 8 - protein, 9 aa, Homo sapiens
NAFIICFYL

SEQ ID NO: 9 - protein, 9 aa, Homo sapiens
YLILYAAWL

SEQ ID NO: 10 - protein, 9 aa, Homo sapiens
VLWVGWNAF

SEQ ID NO: 11 - protein, 9 aa, Homo sapiens
LYAAWLVLW

SEQ ID NO: 12 - protein, 15 aa, Homo sapiens
AWLVLWVGWNAFIIC

SEQ ID NO: 13 - protein, 15 aa, Homo sapiens
LWVGWNAFIICFYLE

SEQ ID NO: 14 - protein, 15 aa, Homo sapiens
AAWLVLWVGWNAFII

SEQ ID NO: 15 - protein, 15 aa, Homo sapiens
VLWVGWNAFIICFYL

SEQ ID NO: 16 - protein, 15 aa, Homo sapiens
WVGWNAFIICFYLEV

SEQ ID NO: 17 - protein, 622 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 18 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 19 - protein, 8 aa, Homo sapiens
PQAPRRPL

SEQ ID NO: 20 - protein, 23 aa, Homo sapiens
VNKGHEMSPQAPRRPLPQVATLI

SEQ ID NO: 21 - protein, 9 aa, Homo sapiens
PLPQVATLI

SEQ ID NO: 22 - protein, 9 aa, Homo sapiens
SPQAPRRPL

SEQ ID NO: 23 - protein, 15 aa, Homo sapiens
VNKGHEMSPQAPRRP

SEQ ID NO: 24 - protein, 15 aa, Homo sapiens
NKGHEMSPQAPRRPL

SEQ ID NO: 25 - protein, 12 aa, Homo sapiens
RPLPQVATLIDR

SEQ ID NO: 26 - protein, 14 aa, Homo sapiens
GHEMSPQVATLIDR

SEQ ID NO: 27 - protein, 11 aa, Homo sapiens
WSDPITLHQGK

SEQ ID NO: 28 - skipped

SEQ ID NO: 29 - protein, 293 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 30 - protein, 247 aa, Homo sapiens
MSGLGRSRRGGRSRVDQEERWSLSCRKEQGKFYDHLLRDCISCASICGQHPKQCAYFCENKLRSPVNLPPELRRQRSGEVENNSDNSGRYQGLEHRGSEASPALPGLKLSADQVALVYSTLGLCLCAVLCCFLVAVACFLKKRGDPCSCQPRSRPRQSPAKSSQDHAMEAGSPVSTSPEPVETCSFCFPECRAPTQESAVTPGTPDPTCAGRWGCHTRTTVLQPCPHIPDSGLGIVCVPAQEGGPGA

SEQ ID NO: 31 - protein, 176 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCISCASICGQHPKQCAYFCENKLRSPVNLPPELRRQRSGEVENNSDNSGRYQGLEHRGSEASPAPRGCPAPGTRKSFWDKENFQGEGFHLG

SEQ ID NO: 32 - protein, 47 aa, Homo sapiens
FPQGLWTGVAMRSCPEEQYWDPLLGTCMSCKTICNHQSQRTCAAFCR

SEQ ID NO: 33 - protein, 393 aa, Homo sapiens [sequence omitted]

SEQ ID NO: 34 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 35 - protein, 2839 aa, Homo sapiens
MAAHRPVEWVQAVVSRFDEQLPIKTGQQNTHTKVSTEHNKECLINISKYKFSLVISGLTTILKNVNNMRIFGEAAEKNLYLSQLIILDTLEKCLAGQPKDTMRLDETMLVKQLLPEICHFLHTCREGNQHAAELRNSASGVLFSLSCNNFNAVFSRISTRLQELTVCSEDNVDVHDIELLQYINVDCAKLKRLLKETAFKFKALKKVAQLAVINSLEKAFWNWVENYPDEFTKLYQIPQTDMAECAEKLFDLVDGFAESTKRKAAVWPLQIILLILCPEIIQDISKDVVDENNMNKKLFLDSLRKALAGHGGSRQLTESAAIACVKLCKASTYINWEDNSVIFLLVQSMVVDLKNLLFNPSKPFSRGSQPADVDLMIDCLVSCFRISPHNNQHFKICLAQNSPSTFHYVLVNSLHRIITNSALDWWPKIDAVYCHSVELRNMFGETLHKAVQGCGAHPAIRMAPSLTFKEKVTSLKFKEKPTDLETRSYKYLLLSMVKLIHADPKLLLCNPRKQGPETQGSTAELITGLVQLVPQSHMPEIAQEAMEALLVLHQLDSIDLWNPDAPVETFWEISSQMLFYICKKLTSHQMLSSTEILKWLREILICRNKFLLKNKQADRSSCHFLLFYGVGCDIPSSGNTSQMSMDHEELLRTPGASLRKGKGNSSMDSAAGCSGTPPICRQAQTKLEVALYMFLWNPDTEAVLVAMSCFRHLCEEADIRCGVDEVSVHNLLPNYNTFMEFASVSNMMSTGRAALQKRVMALLRRIEHPTAGNTEAWEDTHAKWEQATKLILNYPKAKMEDGQAAESLHKTIVKRRMSHVSGGGSIDLSDTDSLQEWINMTGFLCALGGVCLQQRSNSGLATYSPPMGPVSERKGSMISVMSSEGNADTPVSKFMDRLLSLMVCNHEKVGLQIRTNVKDLVGLELSPALYPMLFNKLKNTISKFFDSQGQVLLTDTNTQFVEQTIAIMKNLLDNHTEGSSEHLGQASIETMMLNLVRYVRVLGNMVHAIQIKTKLCQLVEVMMARRDDLSFCQEMKFRNKMVEYLTDWVMGTSNQAADDDVKCLTRDLDQASMEAVVSLLAGLPLQPEEGDGVELMEAKSQLFLKYFTLFMNLLNDCSEVEDESAQTGGRKRGMSRRLASLRHCTVLAMSNLLNANVDSGLMHSIGLGYHKDLQTRATFMEVLTKILQQGTEFDTLAETVLADRFERLVELVTMMGDQGELPIAMALANVVPCSQWDELARVLVTLFDSRHLLYQLLWNMFSKEVELADSMQTLFRGNSLASKIMTFCFKVYGATYLQKLLDPLLRIVITSSDWQHVSFEVDPTRLEPSESLEENQRNLLQMTEKFFHAIISSSSEFPPQLRSVCHCLYQATCHSLLNKATVKEKKENKKSVVSQRFPQNSIGAVGSAMFLRFINPAIVSPYEAGILDKKPPPRIERGLKLMSKILQSIANHVLFTKEEHMRPFNDFVKSNFDAARRFFLDIASDCPTSDAVNHSLSFISDGNVLALHRLLWNNQEKIGQYLSSNRDHKAVGRRPFDKMATLLAYLGPPEHKPVADTHWSSLNLTSSKFEEFMTRHQVHEKEEFKALKTLSIFYQAGTSKAGNPIFYYVARRFKTGQINGDLLIYHVLLTLKPYYAKPYEIVVDLTHTGPSNRFKTDFLSKWFVVFPGFAYDNVSAVYIYNCNSWVREYTKYHERLLTGLKGSKRLVFIDCPGKLAEHIEHEQQKLPAATLALEEDLKVFHNALKLAHKDTKVSIKVGSTAVQVTSAERTKVLGQSVFLNDIYYASEIEEICLVDENQFTLTIANQGTPLTFMHQECEAIVQSIIHIRTRWELSQPDSIPQHTKIRPKDVPGTLLNIALLNLGSSDPSLRSAAYNLLCALTCTFNLKIEGQLLETSGLCIPANNTLFIVSISKTLAANEPHLTLEFLEECISGFSKSSIELKHLCLEYMTPWLSNLVRFCKHNDDAKRQRVTAILDKLITMTINEKQMYPSIQAKIWGSLGQITDLLDVVLDSFIKTSATGGLGSIKAEVMADTAVALASGNVKLVSSKVIGRMCKIIDKTCLSPTPTLEQHLMWDDIAILARYMLMLSFNNSLDVAAHLPYLFHVVTFLVATGPLSLRASTHGLVINIIHSLCTCSQLHFSEETKQVLRLSLTEFSLPKFYLLFGISKVKSAAVIAFRSSYRDRSFSPGSYERETFALTSLETVTEALLEIMEACMRDIPTCKWLDQWTELAQRFAFQYNPSLQPRALVVFGCISKRVSHGQIKQIIRILSKALESCLKGPDTYNSQVLIEATVIALTKLQPLLNKDSPLHKALFWVAVAVLQLDEVNLYSAGTALLEQNLHTLDSLRIFNDKSPEEVFMAIRNPLEWHCKQMDHFVGLNFNSNFNFALVGHLLKGYRHPSPAIVARTVRILHTLLTLVNKHRNCDKFEVNTQSVAYLAALLTVSEEVRSRCSLKHRKSLLLTDISMENVPMDTYPIHHGDPSYRTLKETQPWSSPKGSEGYLAATYPTVGQTSPRARKSMSLDMGQPSQANTKKLLGTRKSFDHLISDTKAPKRQEMESGITTPPKMRRVAETDYEMETQRISSSQQHPHLRKVSVSESNVLLDEEVLTDPKIQALLLTVLATLVKYTTDEFDQRILYEYLAEASVVFPKVFPVVHNLLDSKINTLLSLCQDPNLLNPIHGIVQSVVYHEESPPQYQTSYLQSFGFNGLWRFAGPFSKQTQIPDYAELIVKFLDALIDTYLPGIDEETSEESLLTPTSPYPPALQSQLSITANLNLSNSMTSLATSQHSPGIDKENVELSPTTGHCNSGRTRHGSASQVQKQRSAGSFKRNSIKKIV

SEQ ID NO: 36 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 37 - protein, 403 aa, Homo sapiens [sequence omitted]

SEQ ID NO: 38 - protein, 189 aa, Homo sapiens [sequence omitted]

SEQ ID NO: 39 - protein, 443 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 40 - protein, 1512 aa, Homo sapiens
MAAAAGNRASSSGFPGARATSPEAGGGGGALKASSAPAAAAGLLREAGSGGRERADWRRRQLRKVRSVELDQLPEQPLFLAASPPASSTSPSPEPADAAGSGTGFQPVAVPPPHGAASRGGAHLTESVAAPDSGASSPAAAEPGEKRAPAAEPSPAAAPAGREMENKETLKGLHKMDDRPEERMIREKLKATCMPAWKHEWLERRNRRGPVVVKPIPVKGDGSEMNHLAAESPGEVQASAASPASKGRRSPSPGNSPSGRTVKSESPGVRRKRVSPVPFQSGRITPPRRAPSPDGFSPYSPEETNRRVNKVMRARLYLLQQIGPNSFLIGGDSPDNKYRVFIGPQNCSCARGTFCIHLLFVMLRVFQLEPSDPMLWRKTLKNFEVESLFQKYHSRRSSRIKAPSRNTIQKFVSRMSNSHTLSSSSTSTSSSENSIKDEEEQMCPICLLGMLDEESLTVCEDGCRNKLHHHCMSIWAEECRRNREPLICPLCRSKWRSHDFYSHELSSPVDSPSSLRAAQQQTVQQQPLAGSRRNQESNFNLTHYGTQQIPPAYKDLAEPWIQVFGMELVGCLFSRNWNVREMALRRLSHDVSGALLLANGESTGNSGGSSGSSPSGGATSGSSQTSISGDVVEACCSVLSMVCADPVYKVYVAALKTLRAMLVYTPCHSLAERIKLQRLLQPVVDTILVKCADANSRTSQLSISTLLELCKGQAGELAVGREILKAGSIGIGGVDYVLNCILGNQTESNNWQELLGRLCLIDRLLLEFPAEFYPHIVSTDVSQAEPVEIRYKKLLSLLTFALQSIDNSHSMVGKLSRRIYLSSARMVTTVPHVFSKLLEMLSVSSSTHFTRMRRRLMAIADEVEIAEAIQLGVEDTLDGQQDSFLQASVPNNYLETTENSSPECTVHLEKTGKGLCATKLSASSEDISERLASISVGPSSSTTTTTTTTEQPKPMVQTKGRPHSQCLNSSPLSHHSQLMFPALSTPSSSTPSVPAGTATDVSKHRLQGFIPCRIPSASPQTQRKFSLQFHRNCPENKDSDKLSPVFTQSRPLPSSNIHRPKPSRPTPGNTSKQGDPSKNSMTLDLNSSSKCDDSFGCSSNSSNAVIPSDETVFTPVEEKCRLDVNTELNSSIEDLLEASMPSSDTTVTFKSEVAVLSPEKAENDDTYKDDVNHNQKCKEKMEAEEEEALAIAMAMSASQDALPIVPQLQVENGEDIIIIQQDTPETLPGHTKAKQPYREDTEWLKGQQIGLGAFSSCYQAQDVGTGTLMAVKQVTYVRNTSSEQEEVVEALREEIRMMSHLNHPNIIRMLGATCEKSNYNLFIEWMAGGSVAHLLSKYGAFKESVVINYTEQLLRGLSYLHENQIIHRDVKGANLLIDSTGQRLRIADFGAAARLASKGTGAGEFQGQLLGTIAFMAPEVLRGQQYGRSCDVWSVGCAIIEMACAKPPWNAEKHSNHLALIFKIASATTAPSIPSHLSPGLRDVALRCLELQPQDRPPSRELLKHPVFRTTW

SEQ ID NO: 41 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKVFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 42 - protein, 707 aa, Homo sapiens
MNQELLSVGSKRRRTGGSLRGNPSSSQVDEEQMNRVVEEEQQQQLRQQEEEHTARNGEVVGVEPRPGGQNDSQQGQLEENNNRFISVDEDSSGNQEEQEEDEEHAGEQDEEDEEEEEMDQESDDFDQSDDSSREDEHTHTNSVTNSSSIVDLPVHQLSSPFYTKTTKMKRKLDHGSEVRSFSLGKKPCKVSEYTSTTGLVPCSATPTTFGDLRAANGQGQQRRRITSVQPPTGLQEWLKMFQSWSGPEKLLALDELIDSCEPTQVKHMMQVIEPQFQRDFISLLPKELALYVLSFLEPKDLLQAAQTCRYWRILAEDNLLWREKCKEEGIDEPLHIKRRKVIKPGFIHSPWKSAYIRQHRIDTNWRRGELKSPKVLKGHDDHVITCLQFCGNRIVSGSDDNTLKVWSAVTGKCLRTLVGHTGGVWSSQMRDNIIISGSTDRTLKVWNAETGECIHTLYGHTSTVRCMHLHEKRVVSGSRDATLRVWDIETGQCLHVLMGHVAAVRCVQYDGRRVVSGAYDFMVKVWDPETETCLHTLQGHTNRVYSLQFDGIHVVSGSLDTSIRVWDVETGNCIHTLTGHQSLTSGMELKDNILVSGNADSTVKIWDIKTGQCLQTLQGPNKHQSAVTCLQFNKNFVITSSDDGTVKLWDLKTGEFIRNLVTLESGGSGGVVWRIRASNTKLVCAVGSRNGTEETKLLVLDFDVDMK

SEQ ID NO: 43 - protein, 912 aa, Homo sapiens
MPAMPSSGPGDTSSSAAEREEDRKDGEEQEEPRGKEERQEPSTTARKVGRPGRKRKHPPVESGDTPKDPAVISKSPSMAQDSGASELLPNGDLEKRSEPQPEEGSPAGGQKGGAPAEGEGAAETLPEASRAVENGCCTPKEGRGAPAEAGKEQKETNIESMKMEGSRGRLRGGLGWESSLRQRPMPRLTFQAGDPYYISKRKRDEWLARWKREAEKKAKVIAGMNAVEENQGPGESQKVEEASPPAVQQPTDPASPTVATTPEPVGSDAGDKNATKAGDDEPEYEDGRGFGIGELVWGKLRGFSWWPGRIVSWWMTGRSRAAEGTRWVMWFGDGKFSVVCVEKLMPLSSFCSAFHQATYNKQPMYRKAIYEVLQVASSRAGKLFPVCHDSDESDTAKAVEVQNKPMIEWALGGFQPSGPKGLEPPEEEKNPYKEVYTDMWVEPEAAAYAPPPPAKKPRKSTAEKPKVKEIIDERTRERLVYEVRQKCRNIEDICISCGSLNVTLEHPLFVGGMCQNCKNCFLECAYQYDDDGYQSYCTICCGGREVLMCGNNNCCRCFCVECVDLLVGPGAAQAAIKEDPWNCYMCGHKGTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 44 - protein, 2442 aa, Homo sapiens
MAENLLDGPPNPKRAKLSSPGFSANDSTDFGSLFDLENDLPDELIPNGGELGLLNSGNLVPDAASKHKQLSELLRGGSGSSINPGIGNVSASSPVQQGLGGQAQGQPNSANMASLSAMGKSPLSQGDSSAPSLPKQAASTSGPTPAASQALNPQAQKQVGLATSSPATSQTGPGICMNANFNQTHPGLLNSNSGHSLINQASQGQAQVMNGSLGAAGRGRGAGMPYPTPAMQGASSSVLAETLTQVSPQMTGHAGLNTAQAGGMAKMGITGNTSPFGQPFSQAGGQPMGATGVNPQLASKQSMVNSLPTFPTDIKNTSVTNVPNMSQMQTSVGIVPTQAIATGPTADPEKRKLIQQQLVLLLHAHKCQRREQANGEVRACSLPHCRTMKNVLNHMTHCQAGKACQVAHCASSRQIISHWKNCTRHDCPVCLPLKNASDKRNQQTILGSPASGIQNTIGSVGTGQQNATSLSNPNPIDPSSMQRAYAALGLPYMNQPQTQLQPQVPGQQPAQPQTHQQMRTLNPLGNNPMNIPAGGITTDQQPPNLISESALPTSLGATNPLMNDGSNSGNIGTLSTIPTAAPPSSTGVRKGWHEHVTQDLRSHLVHKLVQAIFPTPDPAALKDRRMENLVAYAKKVEGDMYESANSRDEYYHLLAEKIYKIQKELEEKRRSRLHKQGILGNQPALPAPGAQPPVIPQAQPVRPPNGPLSLPVNRMQVSQGMNSFNPMSLGNVQLPQAPMGPRAASPMNHSVQMNSMGSVPGMAISPSRMPQPPNMMGAHTNNMMAQAPAQSQFLPQNQFPSSSGAMSVGMGQPPAQTGVSQGQVPGAALPNPLNMLGPQASQLPCPPVTQSPLHPTPPPASTAAGMPSLQHTTPPGMTPPQPAAPTQPSTPVSSSGQTPTPTPGSVPSATQTQSTPTVQAAAQAQVTPQPQTPVQPPSVATPQSSQQQPTPVHAQPPGTPLSQAAASIDNRVPTPSSVASAETNSQQPGPDVPVLEMKTETQAEDTEPDPGESKGEPRSEMMEEDLQGASQVKEETDIAEQKSEPMEVDEKKPEVKVEVKEEEESSSNGTASQSTSPSQPRKKIFKPEELRQALMPTLEALYRQDPESLPFRQPVDPQLLGIPDYFDIVKNPMDLSTIKRKLDTGQYQEPWQYVDDVWLMFNNAWLYNRKTSRVYKFCSKLAEVFEQEIDPVMQSLGYCCGRKYEFSPQTLCCYGKQLCTIPRDAAYYSYQNRYHFCEKCFTEIQGENVTLGDDPSQPQTTISKDQFEKKKNDTLDPEPFVDCKECGRKMHQICVLHYDIIWPSGFVCDNCLKKTGRPRKENKFSAKRLQTTRLGNHLEDRVNKFLRRQNHPEAGEVFVRVVASSDKTVEVKPGMKSRFVDSGEMSESFPYRTKALFAFEEIDGVDVCFFGMHVQEYGSDCPPPNTRRVYISYLDSIHFFRPRCLRTAVYHEILIGYLEYVKKLGYVTGHIWACPPSEGDDYIFHCHPPDQKIPKPKRLQEWYKKMLDKAFAERIIHDYKDIFKQATEDRLTSAKELPYFEGDFWPNVLEESIKELEQEEEERKKEESTAASETTEGSQGDSKNAKKKNNKKTNKNKSSISRANKKKPSMPNVSNDLSQKLYATMEKHKEVFFVIHLHAGPVINTLPPIVDPDPLLSCDLMDGRDAFLTLARDKHWEFSSLRRSKWSTLCMLVELHTQGQDRFVYTCNECKHHVETRWHCTVCEDYDLCINCYNTKSHAHKMVKWGLGLDDEGSSQGEPQSKSPQESRRLSIQRCIQSLVHACQCRNANCSLPSCQKMKRVVQHTKGCKRKTNGGCPVCKQLIALCCYHAKHCQENKCPVPFCLNIKHKLRQQQIQHRLQQAQLMRRRMATMNTRNVPQQSLPSPTSAPPGTPTQQPSTPQTPQPPAQPQPSPVSMSPAGFPSVARTQPPTTVSTGKPTSQVPAPPPPAQPPPAAVEAARQIEREAQQQQHLYRVNINNSMPPGRTGMGTPGSQMAPVSLNVPRPNQVSGPVMPSMPPGQWQQAPLPQQQPMPGLPRPVISMQAQAAVAGPRMPSVQPPRSISPSALQDLLRTLKSPSSPQQQQQVLNILKSNPQLMAAFIKQRTAKYVANQPGMQPQPGLQSQPGMQPQPGMHQQPSLQNLNAMQAGVPRPGVPPQQQAMGGLNPQGQALNIMNPGHNPNMASMNPQYREMLRRQLLQQQQQQQQQQQQQQQQQQGSAGMAGGMAGHGQFQQPQGPGGYPPAMQQQQRMQQHLPLQGSSMGQMAAQMGQLGQMGQPGLGADSTPNIQQALQQRILQQQQMKQQIGSPGQPNPMSPQQHMLSGQPQASHLPGQQIATSLSNQVRSPAPVQSPRPQSQPPHSSPSPRIQPQPSPHHVSPQTGSPHPGLAVTMASSIDQGHLGNPEQSAMLPQLNTPSRSALSSELSLVGDTTGDTLEKFVEGL

SEQ ID NO: 45 - protein, 1304 aa, Homo sapiens
MAKIAKTHEDIEAQIREIQGKKAALDEAQGVGLDSTGYYDQEIYGGSDSRFAGYVTSIAATELEDDDDDYSSSTSLLGQKKPGYHAPVALLNDIPQSTEQYDPFAEHRPPKIADREDEYKKHRRTMIISPERLDPFADGGKTPDPKMNARTYMDVMREQHLTKEEREIRQQLAEKAKAGELKVVNGAAASQPPSKRKRRWDQTADQTPGATPKKLSSWDQAETPGHTPSLRWDETPGRAKGSETPGATPGSKIWDPTPSHTPAGAATPGRGDTPGHATPGHGGATSSARKNRWDETPKTERDTPGHGSGWAETPRTDRGGDSIGETPTPGASKRKSRWDETPASQMGGSTPVLTPGKTPIGTPAMNMATPTPGHIMSMTPEQLQAWRWEREIDERNRPLSDEELDAMFPEGYKVLPPPAGYVPIRTPARKLTATPTPLGGMTGFHMQTEDRTMKSVNDQPSGNLPFLKPDDIQYFDKLLVDVDESTLSPEEQKERKIMKLLLKIKNGTPPMRKAALRQITDKAREFGAGPLFNQILPLLMSPTLEDQERHLLVKVIDRILYKLDDLVRPYVHKILVVIEPLLIDEDYYARVEGREIISNLAKAAGLATMISTMRPDIDNMDEYVRNTTARAFAVVASALGIPSLLPFLKAVCKSKKSWQARHTGIKIVQQIAILMGCAILPHLRSLVEIIEHGLVDEQQKVRTISALAIAALAEAATPYGIESFDSVLKPLWKGIRQHRGKGLAAFLKAIGYLIPLMDAEYANYYTREVMLILIREFQSPDEEMKKIVLKVVKQCCGTDGVEANYIKTEILPPFFKHFWQHRMALDRRNYRQLVDTTVELANKVGAAEIISRIVDDLKDEAEQYRKMVMETIEKIMGNLGAADIDHKLEEQLIDGILYAFQEQTTEDSVMLNGFGTVVNALGKRVKPYLPQICGTVLWRLNNKSAKVRQQAADLISRTAVVMKTCQEEKLMGHLGVVLYEYLGEEYPEVLGSILGALKAIVNVIGMHKMTPPIKDLLPRLTPILKNRHEKVQENCIDLVGRIADRGAEYVSAREWMRICFELLELLKAHKKAIRRATVNTFGYIAKAIGPHDVLATLLNNLKVQERQNRVCTTVAIAIVAETCSPFTVLPALMNEYRVPELNVQNGVLKSLSFLFEYIGEMGKDYIYAVTPLLEDALMDRDLVHRQTASAVVQHMSLGVYGFGCEDSLNHLLNYVWPNVFETSPHVIQAVMGALEGLRVAIGPCRMLQYCLQGLFHPARKVRDVYWKIYNSIYIGSQDALIAHYPRIYNDDKNTYIRYELDYIL

SEQ ID NO: 46 - protein, 265 aa, Homo sapiens
MGLPWGQPHLGLQMLLLALNCLRPSLSLELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYREDPRIHRHLARAAKWQHDRHYLHPLFSGRPPTLGLLGSLYHALLQPVVAGGGPGAAADRLLHGQALHDPPHPTQRGRHTAGGLQAWPGPPPQPQPLAWPLCMGLGEMGRWE

SEQ ID NO: 47 - protein, 276 aa, Homo sapiens
MGLPWGQPHLGLQMLLLALNCLRPSLSLELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYRVKFLLMDTRGSPRAETKWSDPITLHQGKTPGSIDTWPGRRSGSMIVITSILSSLAGLLLLAFLAASTMRFSSLWWPEEAPEQLRIGSFMGKRYMTHHIPPSEAATLPVGCKPGLDPLPSLSP

SEQ ID NO: 48 - protein, 320 aa, Homo sapiens
MGLPWGQPHLGLQMLLLALNCLRPSLSLGEWGSWMDASSQTQGAGGPAGVIGPWAPAPLRLGEAAPGTPTPVSVAHLLSPVATELVPYTPQITAWDLEGKVTATTFSLEQPRCVFDGLASASDTVWLVVAFSNASRGFQNPETLADIPASPQLLTDGHYMTLPLSPDQLPCGDPMAGSGGAPVLRVGHDHGCHQQPFCNAPLPGPGPYREDPRIHRHLARAAKWQHDRHYLHPLFSGRPPTLGLLGSLYHALLQPVVAGGGPGAAADRLLHGQALHDPPHPTQRGRHTAGGLQAWPGPPPQPQPLAWPLCMGLGEMGRRE

SEQ ID NO: 49 - protein, 122 aa, Homo sapiens
VKFLLMDTRGSPRAETKWSDPITLHQGKTPGSIDTWPGRRSGSMIVITSILSSLAGLLLLAFLAASTMRFSSLWWPEEAPEQLRIGSFMGKRYMTHHIPPSEAATLPVGCKPGLDPLPSLSP

SEQ ID NO: 50 - protein, 622 aa, Homo sapiens; identical to SEQ ID NO: 17

SEQ ID NO: 51 - protein, 621 aa, Homo sapiens
MALPTARPLLGSCGTPALGSLLFLLFSLGWVQPSRTLAGETGQAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 52 - protein, 612 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQGPGPVLTVLALLLASTLA

SEQ ID NO: 53 - protein, 622 aa, Homo sapiens; identical to SEQ ID NO: 17

SEQ ID NO: 54 - protein, 359 aa, Homo sapiens
MDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQGGRGGQARAGGRAGGVEVGALSHPSLCRGPLGDALPPRTWTCSHRPGTAPSLHPGLRAPLPCWPQPCWGSPPGQEQARVVPVPPQENSRSVNGNMPPADT

SEQ ID NO: 55 - protein, 9 aa, Homo sapiens
ASCVTACPY

SEQ ID NO: 56 - protein, 9 aa, Homo sapiens
LVDAEEYLV

SEQ ID NO: 57 - protein, 9 aa, Homo sapiens
LTCSPQPEY

SEQ ID NO: 58 - protein, 9 aa, Homo sapiens
LIDTNRSRA

SEQ ID NO: 59 - protein, 9 aa, Homo sapiens
GTQLFEDNY

SEQ ID NO: 60 - protein, 9 aa, Homo sapiens
FSPAFDNLY

SEQ ID NO: 61 - protein, 9 aa, Homo sapiens
ETLEEITGY

SEQ ID NO: 62 - protein, 9 aa, Homo sapiens
ETDGYVAPL

SEQ ID NO: 63 - protein, 9 aa, Homo sapiens
STDVGSCTL

SEQ ID NO: 64 - protein, 9 aa, Homo sapiens
QSDVWSYGV

SEQ ID NO: 65 - protein, 9 aa, Homo sapiens
NTDTFESMP

SEQ ID NO: 66 - protein, 9 aa, Homo sapiens
LLDIDETEY

SEQ ID NO: 67 - protein, 9 aa, Homo sapiens
HSDCLACLH

SEQ ID NO: 68 - protein, 9 aa, Homo sapiens
HLDMLRHLY

SEQ ID NO: 69 - protein, 9 aa, Homo sapiens
VMAGVGSPY

SEQ ID NO: 70 - protein, 9 aa, Homo sapiens
TSANIQEFA

SEQ ID NO: 71 - protein, 9 aa, Homo sapiens
FLYTYIAKV

SEQ ID NO: 72 - protein, 9 aa, Homo sapiens
KTLKIVCEV

SEQ ID NO: 73 - protein, 9 aa, Homo sapiens
FLALACSAL

SEQ ID NO: 74 - protein, 9 aa, Homo sapiens
ALACSALGV

SEQ ID NO: 75 - protein, 9 aa, Homo sapiens
FQFLYTYIA

SEQ ID NO: 76 - protein, 9 aa, Homo sapiens
TLNIYLDEL

SEQ ID NO: 77 - protein, 9 aa, Homo sapiens
FLKNHLVFL

SEQ ID NO: 78 - protein, 9 aa, Homo sapiens
HLHCISFLV

SEQ ID NO: 79 - protein, 9 aa, Homo sapiens
RLAPDPSLV

SEQ ID NO: 80 - protein, 9 aa, Homo sapiens
YTFTISQSV

SEQ ID NO: 81 - protein, 9 aa, Homo sapiens
TLTIHTSYV

SEQ ID NO: 82 - protein, 9 aa, Homo sapiens
ALLAYIFSL

SEQ ID NO: 83 - protein, 9 aa, Homo sapiens
LVDYYIDPA

SEQ ID NO: 84 - protein, 9 aa, Homo sapiens
MLALSPAIA

SEQ ID NO: 85 - protein, 9 aa, Homo sapiens
MLGAVQVSV

SEQ ID NO: 86 - protein, 9 aa, Homo sapiens
GLAPFTLET

SEQ ID NO: 87 - protein, 9 aa, Homo sapiens
GIVDLSFQL

SEQ ID NO: 88 - protein, 9 aa, Homo sapiens
LGMLALSPA

SEQ ID NO: 89 - protein, 9 aa, Homo sapiens
ALRAVDESV

SEQ ID NO: 90 - protein, 9 aa, Homo sapiens
MLGTYTVAV

SEQ ID NO: 91 - protein, 9 aa, Homo sapiens
QLAPEAMLG

SEQ ID NO: 92 - protein, 1454 aa, Homo sapiens
MWAQLLLGMLALSPAIAEELPNYLVTLPARLNFPSVQKVCLDLSPGYSDVKFTVTLETKDKTQKLLEYSGLKKRHLHCISFLVPPPAGGTEEVATIRVSGVGNNISFEEKKKVLIQRQGNGTFVQTDKPLYTPGQQVYFRIVTMDSNFVPVNDKYSMVELQDPNSNRIAQWLEVVPEQGIVDLSFQLAPEAMLGTYTVAVAEGKTFGTFSVEEYVLPKFKVEVVEPKELSTVQESFLVKICCRYTYGKPMLGAVQVSVCQKANTYWYREVEREQLPDKCRNLSGQTDKTGCFSAPVDMATFDLIGYAYSHQINIVATVVEEGTGVEANATQNIYISPQMGSMTFEDTSNFYHPNFPFSGKIRVRGHDDSFLKNHLVFLVIYGTNGTFNQTLVTDNNGLAPFTLETSGWNGTDVSLEGKFQMEDLVYNPEQVPRYYQNAYLHLRPFYSTTRSFLGIHRLNGPLKCGQPQEVLVDYYIDPADASPDQEISFSYYLIGKGSLVMEGQKHLNSKKKGLKASFSLSLTFTSRLAPDPSLVIYAIFPSGGVVADKIQFSVEMCFDNQVSLGFSPSQQLPGAEVELQLQAAPGSLCALRAVDESVLLLRPDRELSNRSVYGMFPFWYGHYPYQVAEYDQCPVSGPWDFPQPLIDPMPQGHSSQRSIIWRPSFSEGTDLFSFFRDVGLKILSNAKIKKPVDCSHRSPEYSTAMGAGGGHPEAFESSTPLHQAEDSQVRQYFPETWLWDLFPIGNSGKEAVHVTVPDAITEWKAMSFCTSQSRGFGLSPTVGLTAFKPFFVDLTLPYSVVRGESFRLTATIFNYLKDCIRVQTDLAKSHEYQLESWADSQTSSCLCADDAKTHHWNITAVKLGHINFTISTKILDSNEPCGGQKGFVPQKGRSDTLIKPVLVKPEGVLVEKTHSSLLCPKGKVASESVSLELPVDIVPDSTKAYVTVLGDIMGTALQNLDGLVQMPSGCGEQNMVLFAPIIYVLQYLEKAGLLTEEIRSRAVGFLEIGYQKELMYKHSNGSYSAFGERDGNGNTWLTAFVTKCFGQAQKFIFIDPKNIQDALKWMAGNQLPSGCYANVGNLLHTAMKGGVDDEVSLTAYVTAALLEMGKDVDDPMVSQGLRCLKNSATSTTNLYTQALLAYIFSLAGEMDIRNILLKQLDQQAIISGESIYWSQKPTPSSNASPWSEPAAVDVELTAYALLAQLTKPSLTQKEIAKATSIVAWLAKQHNAYGGFSSTQDTVVALQALAKYATTAYMPSEEINLVVKSTENFQRTFNIQSVNRLVFQQDTLPNVPGMYTLEASGQGCVYVQTVLRYNILPPTNMKTFSLSVEIGKARCEQPTSPRSLTLTIHTSYVGSRSSSNMAIVEVKMLSGFSPMEGTNQLLLQQPLVKKVEFGTDTLNIYLDELIKNTQTYTFTISQSVLVTNLKPATIKVYDYYLPDEQATIQYSDPCE

SEQ ID NO: 93 - protein, 12 aa, Artificial Sequence
  source: /note=“Description of Artificial Sequence: Synthetic peptide”
MPPLLAPLLAPL

SEQ ID NO: 94 - protein, 9 aa, Artificial Sequence
  source: /note=“Description of Artificial Sequence: Synthetic peptide”
MPPLLAPLL

SEQ ID NO: 95 - protein, 9 aa, Artificial Sequence
  source: /note=“Description of Artificial Sequence: Synthetic peptide”
PPLLAPLLA

SEQ ID NO: 96 - protein, 9 aa, Artificial Sequence
  source: /note=“Description of Artificial Sequence: Synthetic peptide”
PLLAPLLAP

SEQ ID NO: 97 - protein, 9 aa, Artificial Sequence
  source: /note=“Description of Artificial Sequence: Synthetic peptide”
LLAPLLAPL

SEQ ID NO: 98 - protein, 91 aa, Homo sapiens
VIQHLGYLFLKMSPEDIRKWNVTSLETLKALLEVNKGHEMSPQVATLIDRFVKGRGQLDKDTLDTLTAFYPGYLCSLSPEELSSVPPSSIW

SEQ ID NO: 99 - protein, 99 aa, Homo sapiens
VIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIW

SEQ ID NO: 100 - protein, 56 aa, Homo sapiens
RGSPRAETKWSDPITLHQGKTPGSIDTWPGRRSGSMIVITSILSSLAGLLLLAFLA

SEQ ID NO: 101 - protein, 48 aa, Homo sapiens
RHLARAAKWQHDRHYLHPLFSGRPPTLGLLGSLYHALLQPVVAGGGPG

SEQ ID NO: 102 - protein, 83 aa, Homo sapiens
PYREDPRIHRHLARAAKWQHDRHYLHPLFSGRPPTLGLLGSLYHALLQPVVAGGGPGAAADRLLHGQALHDPPHPTQRGRHTA

SEQ ID NO: 103 - protein, 93 aa, Homo sapiens
PYRVKFLLMDTRGSPRAETKWSDPITLHQGKTPGSIDTWPGRRSGSMIVITSILSSLAGLLLLAFLAASTMRFSSLWWPEEAPEQLRIGSFMG

SEQ ID NO: 104 - protein, 87 aa, Homo sapiens
MSGLGRSRRGGRSRVDQEERFPQGLWTGVAMRSCPEEQYWDPLLGTCMSCKTICNHQSQRTCAAFCRSLSCRKEQGKFYDHLLRDCI

SEQ ID NO: 105 - protein, 41 aa, Homo sapiens
MSGLGRSRRGGRSRVDQEERWSLSCRKEQGKFYDHLLRDCI